SEQUENCE LISTING (4 sequences)

SEQ ID NO: 1 - protein, 298 aa, Homo sapiens
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 2 - protein, 411 aa, Homo sapiens
MPREKERDAKEPDTMKEESGTDVSVRSRKRKANVAVFLQDPDEEIAKIDRTVRSQCGSQPWDSNRACENPCSLIPTPDKEEDELLYPHAAYKPQRSTPSSSRASPLPVLNWANREEVWKIMLNKEKTYLRDKHLMQRHPLLQPKMRAILLDWLMEVCEVYKLHRETFYLAQDFFDRYMATQQNVVKTLLQLIGISSLFIAAKLEEIYPPKLHQFAYVTDGACSGDEILTMELIIMKALKWHLSPLTIVSWLNVYMQVAYLNDVYEVLLPQYPQQIFIQIAELLDLCVLDVGCLEFSYGVLAASALYHFSSSELMQKVSGYQWCDIEKCVKWMVPFAIVIRETGSSKLKHFRGVPAEDAHNIQTHINSLDLLDKAQAKKAILSEENRISPLPTGVLTPPQSSKKQSSGQGSA

SEQ ID NO: 3 - protein, 404 aa, Homo sapiens
MSRRSSRLQAKQQPQPSQTESPQEAQIIQAKKRKTTQDVKKRREEVTKKHQYEIRNCWPPVLSGGISPCIIIETPHKEIGTSDFSRFTNYRFKNLFINPSPLPDLSWGCSKEVWLNMLKKESRYVHDKHFEVLHSDLEPQMRSILLDWLLEVCEVYTLHRETFYLAQDFFDRFMLTQKDINKNMLQLIGITSLFIASKLEEIYAPKLQEFAYVTDGACSEEDILRMELIILKALKWELCPVTIISWLNLFLQVDALKDAPKVLLPQYSQETFIQIAQLLDLCILAIDSLEFQYRILTAAALCHFTSIEVVKKASGLEWDSISECVDWMVPFVNVVKSTSPVKLKTFKKIPMEDRHNIQTHTNYLAMLEEVNYINTFRKGGQLSPVCNGGIMTPPKSTEKPPGKH

SEQ ID NO: 4 - protein, 432 aa, Homo sapiens
MLGNSAPGPATREAGSALLALQQTALQEDQENINPEKAAPVQQPRTRAALAVLKSGNPRGLAQQQRPKTRRVAPLKDLPVNDEHVTVPPWKANSKQPAFTIHVDEAEKEAQKKPAESQKIEREDALAFNSAISLPGPRKPLVPLDYPMDGSFESPHTMDMSIILEDEKPVSVNEVPDYHEDIHTYLREMEVKCKPKVGYMKKQPDITNSMRAILVDWLVEVGEEYKLQNETLHLAVNYIDRFLSSMSVLRGKLQLVGTAAMLLASKFEEIYPPEVAEFVYITDDTYTKKQVLRMEHLVLKVLTFDLAAPTVNQFLTQYFLHQQPANCKVESLAMFLGELSLIDADPYLKYLPSVIAGAAFHLALYTVTGQSWPESLIRKTGYTLESLKPCLMDLHQTYLKAPQHAQQSIREKYKNSKYHGVSLLNPPETLNL

(19) United States
(12) Patent Application Publication
(10) Pub. No.: US 2026/0200943 A1
(43) Pub. Date: Jul. 16, 2026

(54) COMPOUNDS FOR TREATING CANCER

(71) Applicant: Genesis Therapeutics, Inc., Burlingame, CA (US)

(72) Inventors: Sung Wook Yi, Los Angeles, CA (US); Brandon Reinus, San Diego, CA (US); Advait Nagle, San Diego, CA (US); Evan Nathaniel Feinberg, Palo Alto, CA (US)

(21) Appl. No.: 19/134,950

(22) PCT Filed: Dec. 5, 2023
(86) PCT No.: PCT/US2023/082558

§ 371 Date: Jun. 2, 2025

Related U.S. Application Data
(60) Provisional application No. 63/430,531, filed on Dec. 6, 2022.

(51) Int. Cl. C07D 515/08; A61K 31/4162; A61K 31/439; A61K 31/529; C07D 498/08; C07D 498/18; C07D 515/18

(52) U.S. Cl. CPC C07D 515/08; A61K 31/4162; A61K 31/439; A61K 31/529; C07D 498/08; C07D 498/18; C07D 515/18

(57) ABSTRACT

The present application relates to compounds of Formula (I), as defined herein, and pharmaceutically acceptable salts thereof, as well as processes for preparing compounds of Formula (I), and pharmaceutically acceptable salts thereof. The present application also describes pharmaceutical composition comprising a compound of Formula (I), and pharmaceutically acceptable salts thereof, and methods of using the compounds and compositions for treating diseases, such as cancer.

### CROSS-REFERENCE TO RELATED APPLICATIONS

This application claims the benefit of priority to U.S. Application No. 63/430,531, filed on Dec. 6, 2022, the contents of which are hereby incorporated by reference.

### SEQUENCE LISTING

This application contains a Sequence Listing that has been submitted electronically as an XML file named “49366-0045WO1_SL_ST26.xml”. The XML file, created on Dec. 4, 2023, is 5,957 bytes in size. The material in the XML file is hereby incorporated by reference in its entirety.

### TECHNICAL FIELD

This present application relates to compounds, processes to prepare the compounds, compositions comprising the compounds, and methods of treating disorders (such as cancer) with the compounds or compositions.

### BACKGROUND

Cyclin-dependent kinases (CDKs) perform essential functions in regulating eukaryotic cell division and proliferation. The cyclin-dependent kinase catalytic units are activated by regulatory subunits known as cyclins. At least sixteen mammalian cyclins have been identified. See Johnson, et al., Annu. Rev. Pharmacol. Toxicol. (1999) 39:295-312. Cyclin B/CDK1, cyclin A/CDK2, cyclin E/CDK2, cyclin D/CDK4, cyclin D/CDK6, and likely other heterodynes regulate cell cycle progression. Additional functions of cyclin/CDK heterodynes include regulation of transcription, DNA repair, differentiation and apoptosis. See Morgan D. O., Annu. Rev. Cell. Dev. Biol. (1997) 13:261-291. Overexpression of CDK2 is associated with abnormal regulation of cell-cycle. The cyclin E/CDK2 complex plays and important role in regulation of the G1/S transition, histone biosynthesis and centrosome duplication. Progressive phosphorylation of Rb by cyclin E/Cdk2 releases the G1 transcription factor, E2F, and promotes S-phase entry. Activation of cyclin A/CDK2 during early S-phase promotes phosphorylation of endogenous substrates that permit DNA replication and inactivation of E2F, for S-phase completion. See Asghar et al., Nat. Rev. Drug. Discov. 2015; 14(2): 130-146.

Cyclin E is a regulatory cyclin for CDK2. Amplification or overexpression of cyclin E has long been associated with poor outcomes in breast cancer. See Keyomarsi et al., N Engl J Med. (2002) 347: 1566-75. Cyclin E has at least two types, Cyclin E1 and Cyclin E2. Amplification or overexpression of cyclin E1 (CCNE1) is associated with poor outcomes in ovarian, gastric, endometrial and other cancers. See Nakayama et al., Cancer (2010) 116: 2621-34; Etemadmoghadam et al., Clin. Cancer Res. (2013) 19: 5960-71; Au-Yeung et al., Clin. Cancer Res. (2017) 23: 1862-1874; Ayhan et al., Modern Pathology (2017) 30: 297-303; Ooi et al., Hum. Pathol. (2017) 61: 58-67; Noske et al., Oncotarget (2017) 8: 14794-14805. CDK2 in complex with cyclin E phosphorylates the tumor suppressor RB1 during the G1 phase of the cell cycle. Fully phosphorylated RB1 de-represses the E2F transcription factors which regulate transcription of DNA synthesis and repair genes including cyclin A2. CDK2 complexes with cyclin A2 to phosphorylate and regulate DNA-synthesis/repair processes during S-phase progression.

Amplification or overexpression of cyclin A (CCNA2) is known to be involved in several cancer types, including breast, liver, lung, and cervical. See, e.g., Yam et al., Cell Mol. Life Sci. 2002; 59, 1317-1326 and Burkholm et al., Int. J. Cancer, 2001; 93(2) 283-287. Increased activity of cyclin A is also associated with poor clinical prognosis in non-small cell lung cancer. See Volm, et al., Br. J. Cancer, 1997; 75(12) 1774-1778. Similarly, Cyclin E2 (CCNE2) overexpression is associated with endocrine resistance in breast cancer cells. See Caldon et al., Mol. Cancer Ther. (2012) 11:1488-99; Herrera-Abreu et al., Cancer Res. (2016) 76: 2301-2313. Accordingly, inhibition of CDK2 can provide beneficial effects to cancers associated with aberrations in the cell cycle.

### SUMMARY

Some embodiments provide a compound of Formula (I),

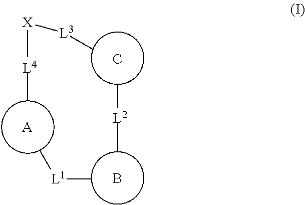

or a pharmaceutically acceptable salt thereof, wherein:
Ring A is a C3-C10 cycloalkyl optionally substituted with 1-3 independently selected R¹;
each R¹ is independently selected from halogen and C1-C6 alkyl;
Ring B is 5-10 membered heteroaryl optionally substituted with 1-3 independently selected R²;
each R² is independently selected from halogen, cyano, C1-C6 alkyl, and C1-C6 haloalkyl;
Ring C is C4-C6 cycloalkyl, 4-6 membered heterocyclyl, phenyl, or 5-6 membered heteroaryl, wherein Ring C is optionally substituted with 1-3 independently selected R³;
each R³ is independently selected from halogen, cyano, C1-C6 alkyl, C1-C6 alkoxy, C1-C6 haloalkyl, C1-C6 haloalkoxy, —NR^{A}R^{B}, —SO₂R^{A}, —NHSO₂R^{A}, and —SO₂NR^{A}R^{B},
each R^{A} and R^{B} is independently selected from hydrogen, C1-C6 alkyl, and C1-C6 haloalkyl; or
R^{A} and R^{B} together with the nitrogen atom to which they are attached form a 4-8 membered heterocyclyl optionally substituted with 1-2 substituents independently selected from halogen and C1-C6 alkyl;
L¹ is a bond;
L² is —NH— or —N(CH₃)—;
L³ is a bond, —NH—, —N(CH₃)—, —O—, *—SO₂NH—, *—NHSO₂—, *—C(═O) NH—, or *—NHC(═O)—, wherein * denotes the point of connection to Ring C;
X is a C2-C15 alkylene, a C4-C15 alkenylene, or a C5-C15 alkynylene, wherein X is optionally substituted with 1-6 independently selected RY and wherein 1-4 methylene units of X are optionally and independently replaced by —O—, —NH—, —N(C1-C6 alkyl)-, —(C3-C6 cycloalkyl)-, or -(5-6 membered heteroaryl)-;
each R^{x} is independently selected from halogen and C1-C6 alkyl;
L⁴ is a bond, *—(CH₂)ₘ—O—C(═O)NH—, or *—(CH₂)ₘ—NHC(═O)NH—, wherein * indicates the point of attachment to Ring A; and
m is 0, 1, or 2.

Some embodiments provide a pharmaceutical composition comprising a compound of Formula (I), or a pharmaceutically acceptable salt thereof, and at least one pharmaceutically acceptable excipient.

Some embodiments provide a method for treating cancer in a subject in need thereof, comprising administering to the subject a therapeutically effective amount of a compound of Formula (I), or a pharmaceutically acceptable salt thereof, or a pharmaceutical composition comprising a compound of Formula (I), or a pharmaceutically acceptable salt thereof, and at least one pharmaceutically acceptable excipient.

Some embodiments provide a method for treating a cancer in a subject in need thereof, comprising: (I) identifying the cancer as being a CDK2-associated cancer; and (b) administering to the subject a therapeutically effective amount of a compound of Formula (I), or a pharmaceutically acceptable salt thereof, or a pharmaceutical composition comprising a compound of Formula (I), or a pharmaceutically acceptable salt thereof, and at least one pharmaceutically acceptable excipient.

Some embodiments provide a method for treating a cancer in a subject in need thereof, comprising: administering to the subject a therapeutically effective amount of a compound of Formula (I), or a pharmaceutically acceptable salt thereof, or a pharmaceutical composition comprising a compound of Formula (I), or a pharmaceutically acceptable salt thereof, and at least one pharmaceutically acceptable excipient, wherein the subject has been identified as having a CDK2-associated cancer.

Some embodiments provide a method of treating a CDK2-associated cancer, comprising administering a therapeutically effective amount of a compound of Formula (I), or a pharmaceutically acceptable salt thereof, or a pharmaceutical composition comprising a compound of Formula (I), or a pharmaceutically acceptable salt thereof, and at least one pharmaceutically acceptable excipient, to a subject identified or diagnosed as having a CDK2-associated cancer.

Some embodiments provide a method for treating cancer in a subject in need thereof, comprising: (I) determining that the cancer is associated with a dysregulation of a CDK2 gene, a CDK2 protein, or expression or activity or level of any of the same; and (b) administering to the subject a therapeutically effective amount of a compound of Formula (I), or a pharmaceutically acceptable salt thereof, or a pharmaceutical composition comprising a compound of Formula (I), or a pharmaceutically acceptable salt thereof, and at least one pharmaceutically acceptable excipient.

Some embodiments provide a method for inhibiting metastasis of a cancer in a subject having a cancer in need of such treatment, comprising administering to the subject a therapeutically effective amount of a compound of Formula (I), or a pharmaceutically acceptable salt thereof, or a pharmaceutical composition comprising a compound of Formula (I), or a pharmaceutically acceptable salt thereof, and at least one pharmaceutically acceptable excipient.

Some embodiments provide a method for inhibiting cancer cell invasiveness in a subject having a cancer in need of such treatment, comprising administering to the subject a therapeutically effective amount of a compound of Formula (I), or a pharmaceutically acceptable salt thereof, or a pharmaceutical composition comprising a compound of Formula (I), or a pharmaceutically acceptable salt thereof, and at least one pharmaceutically acceptable excipient.

Some embodiments provide a method for inhibiting mammalian cell proliferation, comprising contacting the mammalian cell with a compound of Formula (I), or a pharmaceutically acceptable salt thereof.

Some embodiments provide a method for inhibiting CDK2 activity in a mammalian cell, comprising contacting the mammalian cell with a compound of Formula (I), or a pharmaceutically acceptable salt thereof.

Some embodiments provide a method for inducing apoptosis in mammalian cancer cells, comprising contacting the mammalian cell with a compound of Formula (I), or a pharmaceutically acceptable salt thereof.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control.

Other features and advantages of the disclosure will be apparent from the following detailed description and from the claims.

### DETAILED DESCRIPTION

### Definitions

The term “about,” when applied to a specific value or range, refers to +10% of the specified value or range, e.g., to account for experimental variance.

The term “compound,” as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

The term “tautomer,” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer. The following are examples of included tautomeric forms:

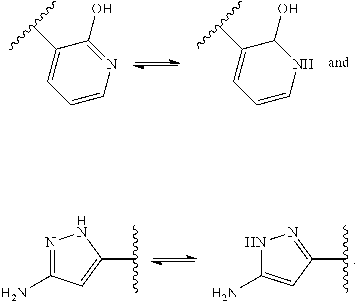 [SMILES: *c1cccnc1O ; *C1=CC=CNC1O ; *c1cc(N)n[nH]1 ; *c1cc(N)[nH]n1]

It will be appreciated that certain compounds provided herein may contain one or more centers of asymmetry and may therefore be prepared and isolated in a mixture of isomers such as a racemic mixture, or in an enantiomerically pure form.

The term “halogen” refers to one of the halogens, group 17 of the periodic table. In particular, the term refers to fluorine, chlorine, bromine and iodine. Preferably, the term refers to fluorine or chlorine.

The term “alkyl” refers to a linear or branched hydrocarbon chain containing from 1-20 carbon atoms. The alkyl group may be denoted as, for example, a C1-12 alkyl group, which contains 1, 2, 3, 4, 5, 6, 7, 8, 9, 10, 11, or 12 carbon atoms. Examples of a C1-C6 alkyl group include methyl, ethyl, n-propyl, iso-propyl, n-butyl, sec-butyl, tert-butyl, n-pentyl and n-hexyl.

When a range of values is listed, it is intended to encompass each value and sub-range within the range. For example “C1-C6 alkyl” is intended to encompass, C1, C2, C3, C4, C5, C6, C1-C6, C1-C5, C1-C4, C1-C3, C1-C2, C2-C6, C2-C5, C2-C4, C2-C3, C3-C6, C3-C5, C3-C4, C4-C6, C4-C5, and C5-C6 alkyl.

The term “alkylene” refers to an alkyl group, as defined herein, which is a biradical and is connected to two other moieties. Non-limiting examples of alkylene groups include: methylene (—CH₂—), ethylene (—CH₂CH₂—), propylene (—CH₂CH₂CH₂—), isopropylene (IUPAC: (methyl)ethylene) (—CH₂—CH(CH₃)—), and isobutylene (IUPAC: 2-(methyl)propylene) (—CH₂—CH(CH₃)—CH₂—). Alkylene groups can optionally include a C3-C6 cycloalkyl, as defined herein, that shares a carbon atom with the backbone of the alkylene chain, for example

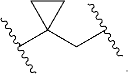 [SMILES: *CC1(*)CC1]

The term “alkenylene” refers to a di-radical of a straight-chain or branched hydrocarbon group having from 2 to 15 carbon atoms, one or more carbon-carbon double bonds, and no triple bonds (“C2-C15 alkenylene”). In some embodiments, one or more carbon-carbon double bonds is cis. In some embodiments, one or more carbon-carbon double bonds is trans. In some embodiments, an alkenylene group has 2 to 10 carbon atoms (“C2-C10 alkenylene”). In some embodiments, an alkenylene group has 2 to 6 carbon atoms (“C2-C6 alkenylene”). In some embodiments, an alkenyl group has 2 to 5 carbon atoms (“C2-C5 alkenylene”). In some embodiments, an alkenylene group has 2 to 4 carbon atoms (“C2-C4 alkenylene”). In some embodiments, an alkenylene group has 2 to 3 carbon atoms (“C2-C3 alkenylene”). In some embodiments, an alkenyl group has 2 carbon atoms (“C2 alkenylene”). The one or more carbon-carbon double bonds can be internal (such as in 2-butenylene) or terminal (such as in 1-butenylene). Examples of C2-C4 alkenyl groups include ethenylene (C2), 1-propenylene (C3), 2-propenylene (C3), 1-butenylene (C4), 2-butenylene (C4), butadienylene (C4), and the like. Examples of C2-C6 alkenylene groups include the aforementioned C2-4 alkenylene groups as well as pentenylene (C5), pentadienylene (C5), hexenylene (C6), and the like. Additional examples of alkenyl include heptenylene (C7), octenylene (C8), octatrienylene (C8), and the like. Each instance of an alkenylene group may be independently optionally substituted, e.g., unsubstituted (an “unsubstituted alkenylene”) or substituted (a “substituted alkenylene”) with one or more substituents, e.g., from 1 to 6 substituents, 1 to 3 substituents, or 1 substituent. In certain embodiments, the alkenylene group is unsubstituted C2-C10 alkenylene. In certain embodiments, the alkenyl group is substituted C2-C6 alkenylene.

The term “alkynylene” refers to a di-radical of a straight-chain or branched hydrocarbon group having from 5 to 15 carbon atoms, one or more carbon-carbon triple bonds, and no double bonds (“C5-C15 alkynylene”). In some embodiments, an alkynylene group has 5 to 14 carbon atoms (“C5-C14 alkynylene”). In some embodiments, an alkynylene group has 5 to 13 carbon atoms (“C5-C13 alkynylene”). In some embodiments, an alkynylene group has 5 to 12 carbon atoms (“C5-C12 alkynylene”). In some embodiments, an alkenylene group has 5 to 11 carbon atoms (“C5-C11 alkynylene”). In some embodiments, an alkynylene group has 5 to 10 carbon atoms (“C5-C10 alkynylene”). In some embodiments, an alkynylene group has 5 to 9 carbon atoms (“C5-C9 alkynylene”). In some embodiments, an alkynylene group has 5 to 8 carbon atoms (“C5-C8 alkynylene”). In some embodiments, an alkynylene group has 5 to 7 carbon atoms (“C5-C7 alkynylene”). In some embodiments, an alkynylene group has 5 to 6 carbon atoms (“C5-C6 alkynylene”). In some embodiments, an alkynylene group has 5 carbon atoms (“C5 alkenylene”). The one or more carbon-carbon triple bonds can be internal (such as in 2-butynyl) or terminal (such as in 1-butynyl). Examples of C5-C10 alkynylene groups include 1-pentynyl (C5), 2-hexynyl (C6), 3-hepynyl (C7), 4-octynyl (C8), 2-nonynyl (C9), 5-decynyl (C10), and the like. Examples of C11-C15 alkynylene groups include, 6-undecynyl (C11), 3-dodecynyl (C12), 7-tridecynyl (C13), 9-tetradecynyl (C14), 2-pentadecynyl (C15), and the like. Additional examples of alkynylene include pentynyl (C5), nonynyl (C9), dodecynyl (C12), and the like. Each instance of an alkynylene group may be independently optionally substituted, e.g., unsubstituted (an “unsubstituted alkynylene”) or substituted (a “substituted alkynylene”) with one or more substituents, e.g., from 1 to 6 substituents, 1 to 3 substituents, or 1 substituent. In certain embodiments, the alkynylene group is unsubstituted C5-C15 alkynylene. In certain embodiments, the alkynylene group is substituted C5-C15 alkynylene.

The term “methylene unit” or “methylene” refers to a C1 di-radical —CH₂—.

The term “haloalkyl” refers to an alkyl group, as defined herein, substituted with at least one halogen atom independently chosen at each occurrence, for example fluorine, chlorine, bromine and iodine. The halogen atom may be present at any position on the hydrocarbon chain. For example, C1-C3 haloalkyl may refer to chloromethyl, fluoromethyl, trifluoromethyl, chloroethyl e.g. 1-chloroethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

The term “alkoxy” refers to an alkyl group, as defined herein, which is attached to a molecule via oxygen. This includes moieties where the alkyl part may be linear or branched, such as methoxy, ethoxy, n-propoxy, iso-propoxy, n-butoxy, sec-butoxy, tert-butoxy, n-pentoxy and n-hexoxy.

As used herein, “haloalkoxy” refers to a O-alkyl group in which one or more of the hydrogen atoms are replaced by a halogen (e.g., mono-haloalkoxy, dihaloalkoxy and tri-haloalkoxy). In some instances, a haloalkoxy can be —OR, wherein R is a C1-4 alkyl substituted by 1, 2 or 3 halogens. Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

As used herein, the term “aryl” refers to a 6-10 all carbon mono- or bicyclic group wherein at least one ring in the system is aromatic, i.e., a C6-C10 aryl. Non-limiting examples of aryl groups include phenyl, naphthyl, tetrahydronaphthyl. In bicyclic ring systems where only one ring is aromatic, the non-aromatic ring can be a cycloalkyl group, as defined herein.

As used herein, the term “heteroaryl” refers to a 5-10 membered mono- or bicyclic group wherein the ring system is aromatic; wherein one or more carbon atoms in at least one ring in the system is/are replaced with an heteroatom independently selected from N, O, and S. Heteroaryl groups include rings where one or more groups are oxidized, such as a pyridone moiety. Non-limiting examples of heteroaryl groups include pyridine, pyrimidine, pyrrole, imidazole, and indole.

As used herein, the term “cycloalkyl” refers to a saturated or partially unsaturated 3-10 mono- or bicyclic hydrocarbon group; wherein bicyclic systems include fused, spiro, and bridged ring systems. Non-limiting examples of cycloalkyl groups include cyclopropyl, cyclohexyl, spiro[2.3]hexyl, and bicyclo[1.1.1]pentyl.

The term “cycloalkoxy” refers to a cycloalkyl group, as defined herein, which is attached to a molecule via oxygen. This includes moieties where the cycloalkyl is saturated or partially unsaturated 3-10 mono- or bicyclic hydrocarbon group; wherein bicyclic systems include fused, spiro, and bridged ring systems. Non-limiting examples of cycloalkoxy groups include cyclopropoxyl, cyclobutoxyl, cyclopentyloxyl, and octahydropentalen-2-yl.

The term “heterocyclyl” refers to a saturated or partially unsaturated 3-12 membered hydrocarbon monocyclic or bicyclic ring system, that is not aromatic, having at least one heteroatom within the ring selected from N, O and S. In bicyclic ring systems, one ring can be aromatic. Bicyclic heterocyclyl groups include fused, spiro, and bridged ring systems. The heterocyclyl ring system may include oxo substitution at one or more C, N, or S ring members. The heterocyclyl group may be denoted as, for example, a “5-10 membered heterocyclyl group,” which is a ring system containing 5, 6, 7, 8, 9 or 10 atoms at least one being a heteroatom. For example, there may be 1, 2 or 3 heteroatoms, optionally 1 or 2. The heterocyclyl group may be bonded to the rest of the molecule through any carbon atom or through a heteroatom such as nitrogen. Exemplary heterocyclyl groups include, but are not limited to, piperidinyl, piperazinyl, morpholino, tetrahydropyranyl, azetidinyl, oxetanyl, 2-azaspiro[3.3]heptanyl, pyrrolidin-2-one, sulfolane, isothiazoline S,S-dioxide, and decahydronaphthalenyl.

The term “hydroxyl” refers to an —OH moiety.

The term “cyano” refers to a —CN moiety.

As used herein, the term “oxo” refers to an “═O” group attached to a carbon atom.

As used herein, an asterisk (*) depicts the point of attachment of an atom or moiety to the indicated atom or group in the remainder of the molecule.

Whenever a group is described herein as being “optionally substituted” that group may be unsubstituted or substituted with one or more of the indicated substituents. Likewise, when a group is described as being “unsubstituted or substituted” if substituted, the substituent(s) may be selected from one or more of the indicated substituents. If no substituents are indicated, it is meant that the indicated “optionally substituted” or “substituted” group may be substituted with one or more group(s) (such as 1, 2 or 3) individually and independently selected from deuterium, alkyl, alkenyl, alkynyl, cycloalkyl, cycloalkenyl, aryl, heteroaryl, heterocyclyl, aryl(alkyl), heteroaryl(alkyl), (heterocyclyl)alkyl, hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanate, nitro, azido, silyl, sulfenyl, sulfinyl, sulfonyl, haloalkyl, haloalkoxy, trihalomethanesulfonyl, trihalomethanesulfonamido, an amino, a mono-substituted amine group and a di-substituted amine group.

The compounds of Formula (I) include pharmaceutically acceptable salts thereof. In addition, the compounds of Formula (I) also include other salts of such compounds which are not necessarily pharmaceutically acceptable salts, and which may be useful as intermediates for preparing and/or purifying compounds of Formula (I) and/or for separating enantiomers of compounds of Formula (I).

The term “pharmaceutically acceptable” indicates that the compound, or salt or composition thereof is compatible chemically and/or toxicologically with the other ingredients comprising a formulation and/or the subject being treated therewith.

Compounds provided herein may also contain unnatural proportions of atomic isotopes at one or more of the atoms that constitute such compounds. That is, an atom, in particular when mentioned in relation to a compound according to Formula (I), comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof; unless expressly noted otherwise. For example, in deuteroalkyl and deuteroalkoxy groups, where one or more hydrogen atoms are specifically replaced with deuterium (²H). As some of the aforementioned isotopes are radioactive, the compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabeled compounds are useful as therapeutic agents, e.g., cancer therapeutic agents, research reagents, e.g., assay reagents, and diagnostic agents, e.g., in vivo imaging agents. All isotopic variations of the compounds provided herein, whether radioactive or not, are intended to be encompassed within the scope of the present disclosure.

The ability of selected compounds to act as CDK2 inhibitors may be demonstrated by the biological assays described herein.

A “CDK2 inhibitor” as defined herein includes any compound exhibiting CDK2 inhibition activity. In some embodiments, a CDK2 inhibitor is selective for a CDK2 protein. Exemplary CDK2 inhibitors can exhibit inhibition activity (Ki) against CDK2 of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, a CDK2 inhibitor can exhibit inhibition activity (Ki) against CDK2 of less than about 25 nM, less than about 10 nM, less than about 5 nM, or less than about 1 nM as measured in an assay as provided herein.

The phrase “therapeutically effective amount” means an amount of compound that, when administered to a subject in need of such treatment, is sufficient to (i) treat a CDK2-associated cancer, (ii) attenuate, ameliorate, or eliminate one or more symptoms of the particular CDK2-associated cancer, and/or (iii) delay the onset of one or more symptoms of the particular CDK2-associated cancer described herein. A therapeutically effective amount can have the effect of, for example, reducing tumor size, inhibiting tumor growth, inhibiting cancer cell invasiveness, inhibiting metastasis, or a combination of any of the foregoing. The amount of a compound of Formula (I), or a pharmaceutically acceptable salt thereof that will correspond to such an amount will vary depending upon factors such as the particular compound, disease condition and its severity, the identity (e.g., weight) of the subject in need of treatment.

Compounds of Formula (I), or a pharmaceutically acceptable salt thereof, are useful for treating diseases and disorders which can be treated with a CDK2 inhibitor, such as CDK2-associated cancers, such as solid tumors.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the term “subject” refers to any animal, including mammals such as humans. In some embodiments, the subject is a human. In some embodiments, the subject has experienced and/or exhibited at least one symptom of the cancer to be treated.

In certain embodiments, compounds of Formula (I), or a pharmaceutically acceptable salt thereof are useful for preventing diseases and disorders as defined herein. The term “preventing” as used herein means the prevention of the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

The term “CDK2-associated cancer” as used herein refers to cancers associated with or having a dysregulation of a CDK2 gene, a CDK2 protein, or the expression or activity or level of any (e.g., one or more) of the same (e.g., any of the types of dysregulation of a CDK2 gene, a CDK2 protein, or the expression or activity or level of any of the same described herein). CDK2-associated cancers also include cancers associated with or having a dysregulation of a cyclin A2 gene, a cyclin A2 protein, or the expression or activity or level of any of the same, cancers associated with or having a dysregulation of a cyclin E1 gene, a cyclin E1 protein, or the expression or activity or level of any of the same, and cancers associated with or having a dysregulation of a cyclin E2 gene, a cyclin E2 protein, or the expression or activity or level of any of the same. In some embodiments, a CDK-associated cancer is characterized by amplification or overexpression of CDK2. In some embodiments, a CDK-associated cancer is characterized by amplification or overexpression of cyclin A2 (CCNA2), cyclin E1 (CCNE1), and/or cyclin E2 (CCNE2). In some embodiments, a CDK-associated cancer is characterized by amplification or overexpression of cyclin E1 (CCNE1) and/or cyclin B2 (CCNE2). In some embodiments, a CDK-associated cancer is characterized by amplification or overexpression of cyclin A2 (CCNA2). In some embodiments, a CDK-associated cancer is characterized by amplification or overexpression of cyclin E1 (CCNE1). In some embodiments, a CDK-associated cancer is characterized by amplification or overexpression of cyclin E2 (CCNE2). Non-limiting examples of a CDK2-associated cancer are described herein.

An exemplary sequence of human CDK2 is shown below:

(UniProt Accession No. P24941)
SEQ ID NO: 1
MENFQKVEKIGEGTYGVVYKARNKLTGEVVALKKIRLDTETEGVPSTAI
REISLLKELNHPNIVKLLDVIHTENKLYLVFEFLHQDLKKEMDASALTG
IPLPLIKSYLFQLLQGLAFCHSHRVLHRDLKPQNLLINTEGAIKLADFG
LARAFGVPVRTYTHEVVTLWYRAPEILLGCKYYSTAVDIWSLGCIFAEM
VTRRALFPGDSEIDQLFRIFRTLGTPDEVVWPGVTSMPDYKPSFPKWAR
QDFSKVVPPLDEDGRSLLSQMLHYDPNKRISAKAALAHPFFQDVTKPVP
HLRL

An exemplary sequence of human Cyclin E1 is shown below:

(UniProt Acession No. E1B9U2)
SEQ ID NO: 2
MPREKERDAKEPDTMKEESGTDVSVRSRKRKANVAVFLQDPDEEIAKID
RTVRSQCGSQPWDSNRACENPCSLIPTPDKEEDELLYPHAAYKPQRSTP
SSSRASPLPVLNWANREEVWKIMLNKEKTYLRDKHLMQRHPLLQPKMRA
ILLDWLMEVCEVYKLHRETFYLAQDFFDRYMATQQNVVKTLLQLIGISS
LFIAAKLEEIYPPKLHQFAYVTDGACSGDEILTMELIIMKALKWHLSPL
TIVSWLNVYMQVAYLNDVYEVLLPQYPQQIFIQIAELLDLCVLDVGCLE
FSYGVLAASALYHESSSELMQKVSGYQWCDIEKCVKWMVPFAIVIRETG
SSKLKHFRGVPAEDAHNIQTHINSLDLLDKAQAKKAILSEENRISPLPT
GVLTPPQSSKKQSSGQGSA

An exemplary sequence of human Cyclin E2 is shown below:

(UniProt Accession No. 096020)
SEQ ID NO: 3
MSRRSSRLQAKQQPQPSQTESPQEAQIIQAKKRKTTQDVKKRREEVTKK
HQYEIRNCWPPVLSGGISPCIIIETPHKEIGTSDFSRFTNYRFKNLFIN
PSPLPDLSWGCSKEVWLNMLKKESRYVHDKHFEVLHSDLEPQMRSILLD
WLLEVCEVYTLHRETFYLAQDFFDREMLTQKDINKNMLQLIGITSLFIA
SKLEEIYAPKLQEFAYVTDGACSEEDILRMELIILKALKWELCPVTIIS
WLNLFLQVDALKDAPKVLLPQYSQETFIQIAQLLDLCILAIDSLEFQYR
ILTAAALCHFTSIEVVKKASGLEWDSISECVDWMVPFVNVVKSTSPVKL
KTFKKIPMEDRHNIQTHTNYLAMLEEVNYINTERKGGQLSPVCNGGIMT
PPKSTEKPPGKH

An exemplary sequence of human Cyclin A2 is shown below:

(UniProt Accession No. P20248)
SEQ ID NO: 4
MLGNSAPGPATREAGSALLALQQTALQEDQENINPEKAAPVQQPRTRAA
LAVLKSGNPRGLAQQQRPKTRRVAPLKDLPVNDEHVTVPPWKANSKQPA
FTIHVDEAEKEAQKKPAESQKIEREDALAFNSAISLPGPRKPLVPLDYP
MDGSFESPHIMDMSIILEDEKPVSVNEVPDYHEDIHTYLREMEVKCKPK
VGYMKKQPDITNSMRAILVDWLVEVGEEYKLQNETLHLAVNYIDRFLSS
MSVLRGKLQLVGTAAMLLASKFEEIYPPEVAEFVYITDDTYTKKQVLRM
EHLVLKVLTFDLAAPTVNQFLTQYFLHQQPANCKVESLAMFLGELSLID
ADPYLKYLPSVIAGAAFHLALYTVTGQSWPESLIRKTGYTLESLKPCLM
DLHQTYLKAPQHAQQSIREKYKNSKYHGVSLLNPPETLNL

### Compounds of Formula (I)

Provided herein are compounds of Formula (I),

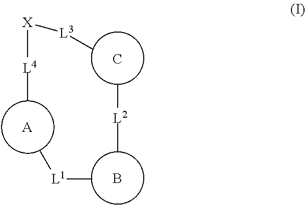

or a pharmaceutically acceptable salt thereof, wherein:
Ring A is a C3-C10 cycloalkyl optionally substituted with 1-3 independently selected R¹; each R¹ is independently selected from halogen and C1-C6 alkyl;
Ring B is 5-10 membered heteroaryl optionally substituted with 1-3 independently selected R²;
each R² is independently selected from halogen, cyano, C1-C6 alkyl, and C1-C6 haloalkyl;
Ring C is C4-C6 cycloalkyl, 4-6 membered heterocyclyl, phenyl, or 5-6 membered heteroaryl, wherein Ring C is optionally substituted with 1-3 independently selected R³;
each R³ is independently selected from halogen, cyano, C1-C6 alkyl, C1-C6 alkoxy, C1-C6 haloalkyl, C1-C6 haloalkoxy, —NR^{A}R^{B}, —SO₂R^{A}, —NHSO₂R^{A}, and —SO₂NR^{A}R^{B};
each R^{A} and R^{B} is independently selected from hydrogen, C1-C6 alkyl, and C1-C6 haloalkyl; or
R^{A} and R^{B} together with the nitrogen atom to which they are attached form a 4-8 membered heterocyclyl optionally substituted with 1-2 substituents independently selected from halogen and C1-C6 alkyl;
L¹ is a bond;
L² is —NH— or —N(CH₃)—;
L³ is a bond, —NH—, —N(CH₃)—, —O—, *—SO₂NH—, *—NHSO₂—, *—C(═O)NH—, or *—NHC(═O)—, wherein * denotes the point of connection to Ring C;
X is a C2-C15 alkylene, a C4-C15 alkenylene, or a C5-C15 alkynylene, wherein X is optionally substituted with 1-6 independently selected R^{x} and wherein 1-4 methylene units of X are optionally and independently replaced by —O—, —NH—, —N(C1-C6 alkyl)-, —(C3-C6 cycloalkyl)-, or -(5-6 membered heteroaryl)-;
each R^{x} is independently selected from halogen and C1-C6 alkyl;
L⁴ is a bond, *—(CH₂)ₘ—O—C(═O)NH—, or *—(CH₂)ₘ—NHC(═O)NH—, wherein * indicates the point of attachment to Ring A; and
m is 0, 1, or 2.

In some embodiments, Ring A is C3-C10 cycloalkyl substituted with 1-3 independently selected R¹. In some embodiments, Ring A is C3-C8 cycloalkyl substituted with 1-3 independently selected R¹. In some embodiments, Ring A is cyclobutyl, cyclopentyl, or [1,1,1]bicyclopentyl substituted with 1-3 independently selected R¹. In some embodiments, Ring A is C3-C10 cycloalkyl substituted with 1 or 2 independently selected R¹. In some embodiments, Ring A is C3-C8 cycloalkyl substituted with 1 or 2 independently selected R¹. In some embodiments, Ring A is cyclobutyl, cyclopentyl, or [1,1,1]bicyclopentyl substituted with 1 or 2 independently selected R¹. In some embodiments, Ring A is C3-C10 cycloalkyl substituted with R¹. In some embodiments, Ring A is C3-C8 cycloalkyl substituted with R¹. In some embodiments, Ring A is cyclobutyl, cyclopentyl, or [1,1,1]bicyclopentyl substituted with R¹.

In some embodiments, Ring A is an unsubstituted C3-C10 cycloalkyl. In some embodiments, Ring A is an unsubstituted C3-C8 cycloalkyl. In some embodiments, Ring A is cyclobutyl, cyclopentyl, [1,1,1]bicyclopentyl, or cyclohexyl. In some embodiments, Ring A is

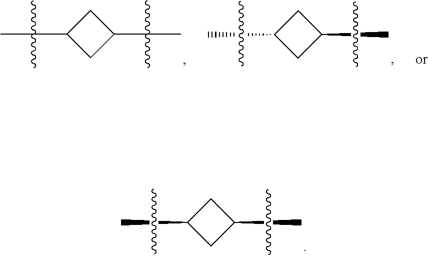

In some embodiments, Ring A is

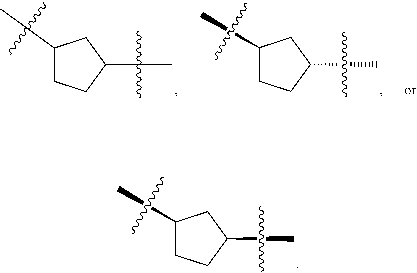

In some embodiments, Ring A is

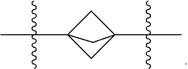 [SMILES: *C12CC(*)(C1)C2]

In some embodiments, Ring B is 5-10 membered heteroaryl substituted with 1-3 independently selected R². In some embodiments, Ring B is 5-10 membered heteroaryl substituted with 1 or 2 independently selected R². In some embodiments, Ring B is 5-10 membered heteroaryl substituted with R². In some embodiments, Ring B is an unsubstituted 5-10 membered heteroaryl.

In some embodiments, Ring B is a 9 membered heteroaryl substituted with 1-3 independently selected R². In some embodiments, Ring B is a 9 membered heteroaryl substituted with 1 or 2 independently selected R². In some embodiments, Ring B is a 9 membered heteroaryl substituted with R². In some embodiments, Ring B is an unsubstituted 9 membered heteroaryl.

In some embodiments, Ring B is 5-6 membered heteroaryl substituted with 1-3 independently selected R². In some embodiments, Ring B is 5-6 membered heteroaryl substituted with 1 or 2 independently selected R². In some embodiments, Ring B is 5-6 membered heteroaryl substituted with R². In some embodiments, Ring B is an unsubstituted 5-6 membered heteroaryl.

In some embodiments, the heteroaryl of Ring B is a 5 membered heteroaryl. In some embodiments, the heteroaryl of Ring B is selected from the group consisting of pyrrolyl, pyrazolyl, imidazolyl, triazolyl, tetrazolyl, furanyl, thiophenyl, oxazolyl, isoxazolyl, isothiazolyl, thiazolyl, furazanyl, oxadiazolyl, thiadiazolyl, oxatriazolyl, and thiatriazolyl.

In some embodiments, Ring B is pyrazolyl. In some embodiments, Ring B is thiazolyl. In some embodiments, Ring B is triazolyl.

In some embodiments, Ring B is

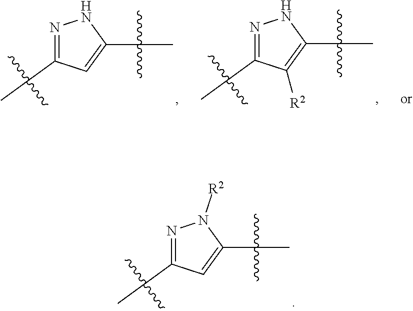

In some embodiments, Ring B is

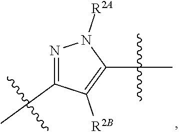

wherein R^{2A} and R^{2B} are each independently selected from R².

In some embodiments, the heteroaryl of Ring B is a 6 membered heteroaryl. In some embodiments, the heteroaryl of Ring B is selected from the group consisting of pyridinyl, pyrimidinyl, pyrazinyl, pyridazinyl, and triazinyl.

In some embodiments, the heteroaryl of Ring B is pyrimidinyl. In some embodiments, Ring B is

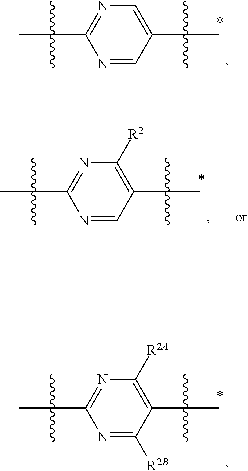

wherein R^{2A} and R^{2B} are each independently selected from R², and * indicated the point of attachment to Ring A.

In some embodiments, Ring C is C4-C6 cycloalkyl substituted with 1-3 independently selected R³. In some embodiments, Ring C is C4-C6 cycloalkyl substituted with 1 or 2 independently selected R³. In some embodiments, Ring C is C4-C6 cycloalkyl substituted with R³. In some embodiments, Ring C is an unsubstituted C4-C6 cycloalkyl. In some embodiments, Ring C is cyclobutyl. In some embodiments, Ring C is cyclopentyl or cyclohexyl.

In some embodiments, Ring C is 4-6 membered heterocyclyl substituted with 1-3 independently selected R³. In some embodiments, Ring C is 4-6 membered heterocyclyl substituted with 1 or 2 independently selected R³. In some embodiments, Ring C is 4-6 membered heterocyclyl substituted with R³. In some embodiments, Ring C is an unsubstituted 4-6 membered heterocyclyl.

In some embodiments, the heterocyclyl of Ring C is a 4 membered heterocyclyl. In some embodiments, the heterocyclyl of Ring C is selected from the group consisting of oxetanyl and azetidinyl.

In some embodiments, the heterocyclyl of Ring C is a 5 membered heterocyclyl. In some embodiments, the heterocyclyl of Ring C is selected from the group consisting of pyrrolidinyl, tetrahydrofuryl, thiolanyl, pyrazolinyl, oxathiolanyl, isoxazolidinyl, isothiazolidinyl, pyrrolinyl, pyrrolidinonyl, pyrazolidinyl, imidazolinyl, dioxolanyl, sulfolanyl, thiazolidedionyl, succinimidyl, dihydrofuranonyl, pyrazolidinonyl, oxazolidinyl, isoxazolidinonyl, hydantionyl, thiohydantionyl, imidazolidinonyl, oxazolidinonyl, thiazolidinonyl, oxathiolanonyl, dioxolanonyl, dioxazolidinonyl, oxadiazolidinonyl, triazolidinonyl, triazolidinethionyl, oxadiazolidinethionyl, dioxazolidinethionyl, dioxolanethionyl, oxazolidinethionyl, imidazolidinethionyl, and isothiazolidinonyl.

In some embodiments, the heterocyclyl of Ring C is a 6 membered heterocyclyl. In some embodiments, the heterocyclyl of Ring C is selected from the group consisting of piperidinyl, tetrahydropyranyl, thianyl, morpholinyl, thiomorpholinyl, dioxanyl, piperazinyl, dithianyl, oxazinyl, tetrahydropyranonyl, piperidinonyl, dioxanonyl, oxazinanonyl, morpholinonyl, thiomorpholinonyl, piperazinonyl, tetrahydropyrimidinonyl, piperidinedionyl, oxazinanedionyl, dihydropyrimidindione, tetrahydropyridazinonyl, triazinanonyl, oxadiazinanonyl, dioxazinanonyl, morpholinedionyl, piperazinedionyl, piperazinetrionyl, and triazinanedionyl.

In some embodiments, the heterocyclyl of Ring C is piperidinyl. In some embodiments, Ring C is

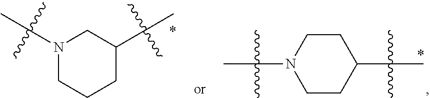 [SMILES: *C1CCCN(*)C1 ; *C1CCN(*)CC1]

wherein * indicated the point of attachment to L².

In some embodiments, Ring C is phenyl substituted with 1-3 independently selected R³. In some embodiments, Ring C is phenyl substituted with 1 or 2 independently selected R³. In some embodiments, Ring C is phenyl substituted with R³.

In some embodiments, Ring C is

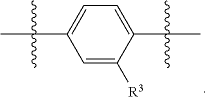

In some embodiments, Ring C is

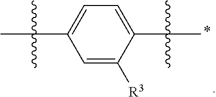

wherein * indicates the point of attachment to L². In some embodiments, Ring C is

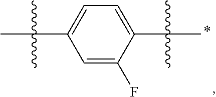 [SMILES: *c1ccc(*)c(F)c1]

wherein * indicates the point of attachment to L².

In some embodiments, Ring C is

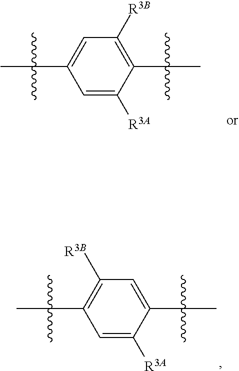

wherein R^{3A} and R^{3B} are independently selected from R³. In some embodiments, Ring C is

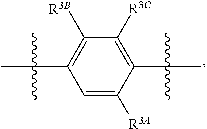

wherein R^{3A}, R^{3B}, and R^{3C} are independently selected from R³.

In some embodiments, Ring C is

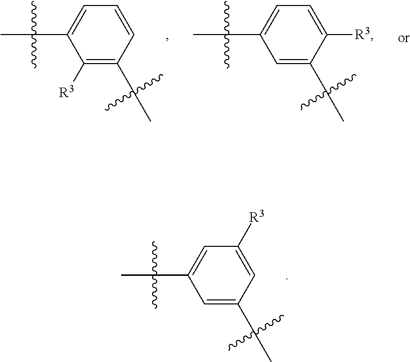

In some embodiments, Ring C is

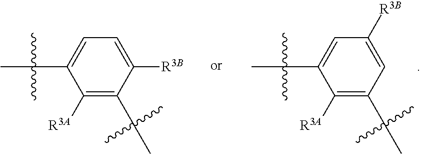

wherein R^{3A} and R^{3B} are independently selected from R³. In some embodiments, Ring C is

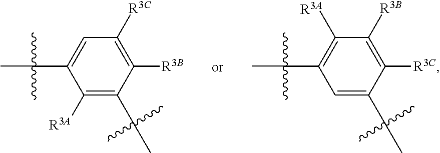

wherein R^{3A}, R^{3B}, and R^{3C} are independently selected from R³.

In some embodiments, Ring C is

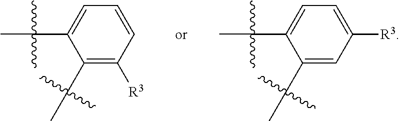

In some embodiments, Ring C is

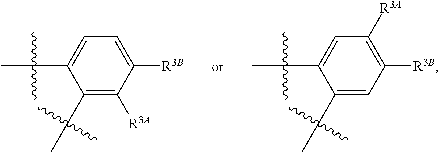

wherein R^{3A} and R^{3B} are independently selected from R³. In some embodiments, Ring C is

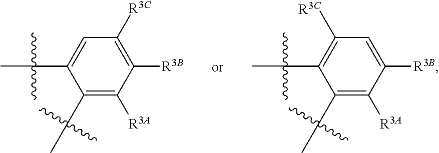

wherein R^{3A}, R^{3B}, and R^{3C} are independently selected from R³.

In some embodiments, Ring C is an unsubstituted phenyl. In some embodiments, Ring C is

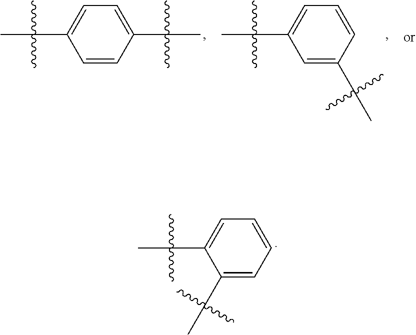 [SMILES: *c1ccc(*)cc1 ; *c1cccc(*)c1 ; *c1ccccc1*]

In some embodiments, Ring C is 5-6 membered heteroaryl substituted with 1-3 independently selected R³, for example 1 R³, 2 R³, or 3 R³. In some embodiments, Ring C is an unsubstituted 5-6 membered heteroaryl.

In some embodiments, Ring C is 5 membered heteroaryl. In some embodiments, Ring C is selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl.

In some embodiments, Ring C is 6 membered heteroaryl. In some embodiments, Ring C is selected from the group consisting of pyridinyl, pyrimidinyl, pyrazinyl, pyridazinyl, and triazinyl.

In some embodiments, Ring C is pyridinyl. In some embodiments, Ring C is

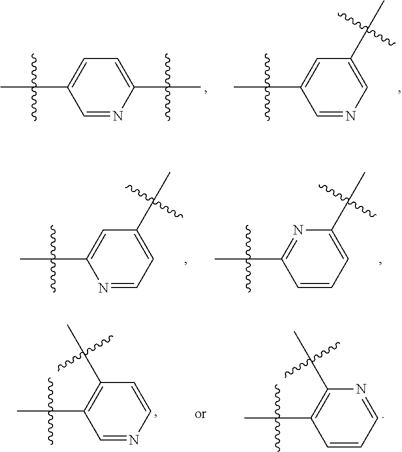 [SMILES: *c1ccc(*)nc1 ; *c1cncc(*)c1 ; *c1ccnc(*)c1 ; *c1cccc(*)n1 ; *c1ccncc1* ; *c1cccnc1*]

In some embodiments, Ring C is

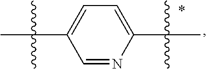 [SMILES: *c1ccc(*)nc1]

wherein * indicates the point of attachment to L².

In some embodiments, R¹ is halogen. In some embodiments, R¹ is —Cl, or —F.

In some embodiments, R¹ is C1-C6 alkyl. In some embodiments, R¹ is C1-C4 alkyl. In some embodiments, R¹ is methyl, ethyl, n-propyl, isopropyl, n-butyl, isobutyl, sec-butyl, or tert-butyl.

In some embodiments, R² is halogen. In some embodiments, R² is —Cl, or —F. In some embodiments, R² is cyano.

In some embodiments, R² is C1-C6 alkyl. In some embodiments, R² is C1-C4 alkyl. In some embodiments, R² is methyl, ethyl, n-propyl, isopropyl, n-butyl, isobutyl, sec-butyl, or tert-butyl.

In some embodiments, R² is C1-C6 haloalkyl. In some embodiments, R² is C1-C3 haloalkyl. In some embodiments, R² is —CF₃, —CF₂H, —CH₂F, —CH₂CF₃, —CH₂CF₂H, or CH₂CH₂F.

In some embodiments, R³ is halogen. In some embodiments, R³ is —F. In some embodiments, R³ is —Cl.

In some embodiments, R³ is cyano.

In some embodiments, R³ is C1-C6 alkyl. In some embodiments, R³ is C1-C4 alkyl. In some embodiments, R² is methyl, ethyl, n-propyl, isopropyl, n-butyl, isobutyl, sec-butyl, or tert-butyl.

In some embodiments, R³ is C1-C6 alkoxy. In some embodiments, R³ is C1-C3 alkoxy. In some embodiments, R³ is —OMe, —OEt, —OiPr.

In some embodiments, R³ is C1-C6 haloalkyl. In some embodiments, R³ is C1-C3 haloalkyl. In some embodiments, R³ is —CF₃, —CF₂H, —CH₂F, —CH₂CF₃, —CH₂CF₂H, or CH₂CH₂F.

In some embodiments, R³ is C1-C6 haloalkoxy. In some embodiments, R³ is C1-C3 haloalkoxy. In some embodiments, R³ is —OCF₂H, —OCF₃, or —OCH₂CF₃.

In some embodiments, R³ is —SO₂R^{A}.

In some embodiments, R³ is —NHSO₂R^{A}.

In some embodiments, R³ is —NR^{A}R^{B}.

In some embodiments, R³ is —SO₂NR^{A}R^{B}. In some embodiments, R³ is —SO₂NH₂.

In some embodiments, R^{A} is hydrogen.

In some embodiments, R^{A} is C1-C6 alkyl. In some embodiments, R^{A} is C1-C4 alkyl. In some embodiments, R² is methyl, ethyl, n-propyl, isopropyl, n-butyl, isobutyl, sec-butyl, or tert-butyl.

In some embodiments, R^{A} is C1-C6 haloalkyl. In some embodiments, R^{A} is C1-C3 haloalkyl. In some embodiments, R^{A} is —CF₃, —CF₂H, —CH₂F, —CH₂CF₃, —CH₂CF₂H, or CH₂CH₂F.

In some embodiments, R^{A} and R^{B} are both hydrogen.

In some embodiments, R^{A} and R^{B} are independently selected C1-C6 alkyl. In some embodiments, R^{A} and R^{B} are independently selected C1-C3 alkyl.

In some embodiments, R^{A} and R^{B} are independently selected C1-C6 haloalkyl. In some embodiments, R^{A} and R^{B} are independently selected C1-C3 haloalkyl.

In some embodiments, one of R^{A} and R^{B} is hydrogen and the other one of R^{A} and R^{B} is C1-C6 alkyl. In some embodiments, one of R^{A} and R^{B} is hydrogen and the other one of R^{A} and R^{B} is C1-C3 alkyl.

In some embodiments, one of R^{A} and R^{B} is hydrogen and the other one of R^{A} and R^{B} is C1-C6 haloalkyl. In some embodiments, one of R^{A} and R^{B} is hydrogen and the other one of R^{A} and R^{B} is C1-C3 haloalkyl.

In some embodiments, one of R^{A} and R^{B} is C1-C6 alkyl and the other one of R^{A} and R^{B} is C1-C6 haloalkyl. In some embodiments, one of R^{A} and R^{B} is C1-C3 alkyl and the other one of R^{A} and R^{B} is C1-C3 haloalkyl.

In some embodiments, R^{A} and R^{B} together with the nitrogen atom to which they are attached form a 4-8 membered heterocyclyl optionally substituted with 1-2 substituents independently selected from halogen and C1-C6 alkyl.

In some embodiments, L² is —NH—. In some embodiments, L² is —N(CH₃)—.

In some embodiments, L³ is a bond.

In some embodiments, L³ is —NH—. In some embodiments, L³ is —N(CH₃)—.

In some embodiments, L³ is —O—.

In some embodiments, L³ is *—SO₂NH—, wherein * denotes the point of connection to Ring C.

In some embodiments, L³ is *—NHSO₂—, wherein * denotes the point of connection to Ring C.

In some embodiments, L³ is *—C(═O)NH—, wherein * denotes the point of connection to Ring C.

In some embodiments, L³ is *—NHC(═O)—, wherein * denotes the point of connection to Ring C.

In some embodiments, L⁴ is a bond.

In some embodiments, L⁴ is *—(CH₂)ₘ—O—C(═O)NH—, wherein * denotes the point of connection to Ring A. In some embodiments, m is 2, wherein L⁴ is *—CH₂CH₂—O—C(═O)NH—, where * denotes the point of connection to Ring A. In some embodiments, m is 1, wherein L⁴ is *—CH₂—O—C(═O)NH—, where * denotes the point of connection to Ring A. In some embodiments, m is 0, wherein L⁴ is *—O—C(═O)NH—, where * denotes the point of connection to Ring A.

In some embodiments, L⁴ is *—(CH₂)ₘ—NHC(═O)NH—, wherein * denotes the point of connection to Ring A. In some embodiments, m is 2, wherein L⁴ is *—CH₂CH₂—NHC(═O) NH—, where * denotes the point of connection to Ring A. In some embodiments, m is 1, wherein L⁴ is *—CH₂—NHC(═O) NH—, where * denotes the point of connection to Ring A. In some embodiments, m is 0, wherein L⁴ is *—NHC(═O) NH—, wherein * denotes the point of connection to Ring A.

In some embodiments, X is a C2-C15 alkylene, a C4-C15 alkenylene, or a C5-C15 alkynylene, substituted with 1-6 independently selected RY and wherein 1-4 methylene units of X are optionally and independently replaced by —O—, —NH—, —N(C1-C6 alkyl)-, —(C3-C6 cycloalkyl)-, or -(5-6 membered heteroaryl)-.

In some embodiments, X is a C2-C15 alkylene, a C4-C15 alkenylene, or a C5-C15 alkynylene, substituted with 1-6 independently selected RY and wherein 1-4 methylene units of X are independently replaced by —O—, —NH—, —N(C1-C6 alkyl)-, —(C3-C6 cycloalkyl)-, or -(5-6 membered heteroaryl)-.

In some embodiments, X is an unsubstituted C2-C15 alkylene, an unsubstituted C4-C15 alkenylene, or an unsubstituted C5-C15 alkynylene, and wherein 1-4 methylene units of X are optionally and independently replaced by —O—, —NH—, —N(C1-C6 alkyl)-, —(C3-C6 cycloalkyl)-, or -(5-6 membered heteroaryl)-.

In some embodiments, X is an unsubstituted C2-C15 alkylene, an unsubstituted C4-C15 alkenylene, or an unsubstituted C5-C15 alkynylene, and wherein 1-4 methylene units of X are independently replaced by —O—, —NH—, —N(C1-C6 alkyl)-, —(C3-C6 cycloalkyl)-, or -(5-6 membered heteroaryl)-.

In some embodiments, X is a C2-C15 alkylene, a C4-C15 alkenylene, or a C5-C15 alkynylene, wherein X is substituted with 1-3 independently selected R^{x}. In some embodiments, X is a C2-C15 alkylene, a C4-C15 alkenylene, or a C5-C15 alkynylene, wherein X is substituted with 2-3 independently selected R^{x} wherein two R^{x} are geminal.

In some embodiments, 1-2 methylene units of X are optionally and independently replaced by —O—, —NH—, —N(C1-C6 alkyl)-, —(C3-C6 cycloalkyl)-, or -(5-6 membered heteroaryl)-. In some embodiments, one methylene unit of X is replaced by —O— or —NH— and second methylene units of X is replaced by -(5-6 membered heteroaryl)-. In some embodiments, one methylene unit of X is replaced by —O— or —NH— and second methylene units of X is replaced by -(5 membered heteroaryl)-. In some embodiments, one methylene unit of X is replaced by —O— or —NH— and second methylene units of X is replaced by -(6 membered heteroaryl)-. In some embodiments, one methylene unit of X is replaced by —O— and second methylene units of X is replaced by -(pyridyl)-. In some embodiments, X is

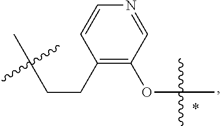 [SMILES: *CCc1ccncc1O*]

wherein * indicates the point of attachment to L⁴.

In some embodiments, one methylene unit of X is replaced by —(C3-C6 cycloalkyl)-. In some embodiments, one methylene unit of X is replaced by —(C3-C4 cycloalkyl)-. In some embodiments, one methylene unit of X is replaced by -(cyclopropyl)-. In some embodiments, one methylene unit of X is replaced by

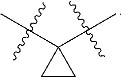 [SMILES: *C1(*)CC1]

In some embodiments, X is

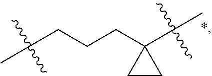 [SMILES: *CCCC1(*)CC1]

wherein * indicates the point of attachment to L⁴.

In some embodiments, X is a C2-C15 alkylene. In some embodiments, X is a C2-C10 alkylene. In some embodiments, X is a C2-C6 alkylene. In some embodiments, X is a C2-C4 alkylene. In some embodiments, X is selected from the group consisting of

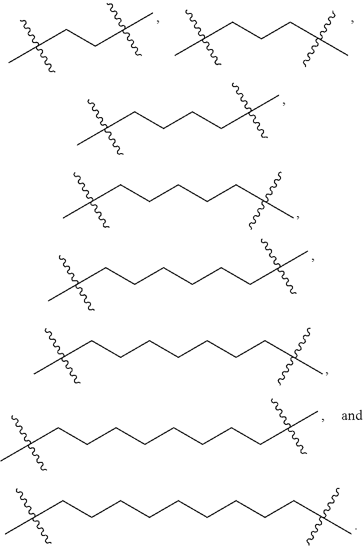

In some embodiments, X is

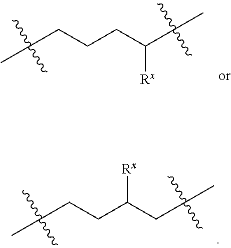

In some embodiments, X is

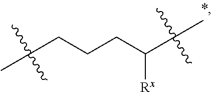

wherein * indicates the point of attachment to L⁴. In some embodiments, X is

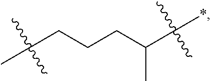

wherein * indicates the point of attachment to L⁴.

In some embodiments, X is

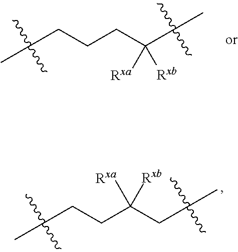

wherein R^{xa} and R^{xb}, are independently selected from R^{x}. In some embodiments, X is

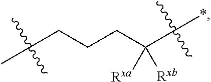

wherein R^{xa} and R^{xb}, are independently selected from R^{x}, and * indicates the point of attachment to L⁴. In some embodiments, X is

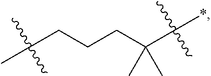

wherein * indicates the point of attachment to L⁴.

In some embodiments, X is a C4-C15 alkenylene. In some embodiments, X is a C4-C10 alkenylene. In some embodiments, X is a C4-C6 alkenylene. In some embodiments, X is a C4-C15 alkenylene that contains trans-alkenyl. In some embodiments, X is a C4-C15 alkenylene that contains cis-alkenyl. In some embodiments, X is a C4-C15 alkenylene that contains both cis- and trans-alkenyl.

In some embodiments, X is a C5-C15 alkynylene. In some embodiments, X is a C5-C10 alkynylene. In some embodiments, X is a C5-C6 alkynylene.

In some embodiments, one R^{x} is halogen.

In some embodiments, one R^{x} is C1-C6 alkyl. In some embodiments, one R^{x} is C1-C3 alkyl. In some embodiments, one R^{x} is methyl.

In some embodiments, two R^{x} are independently selected C1-C6 alkyl. In some embodiments, two R^{x} are independently selected C1-C3 alkyl. In some embodiments, two R^{x} are methyl.

In some embodiments, the compound is a compound of Formula (I-a):

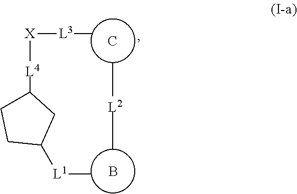

or a pharmaceutically acceptable salt thereof.

In some embodiments, the compound is a compound of Formula (I-b):

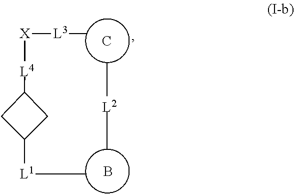

or a pharmaceutically acceptable salt thereof.

In some embodiments, the compound is a compound of Formula (I-c):

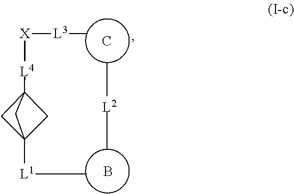

or a pharmaceutically acceptable salt thereof.

In some embodiments, the compound is a compound of Formula (I-d):

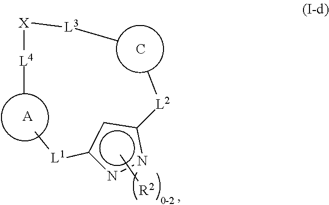

or a pharmaceutically acceptable salt thereof.

In some embodiments, the compound is a compound of Formula (I-e):

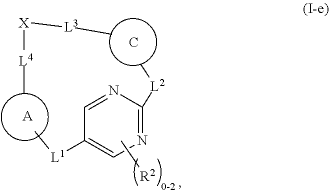

or a pharmaceutically acceptable salt thereof.

In some embodiments, the compound is a compound of Formula (I-f):

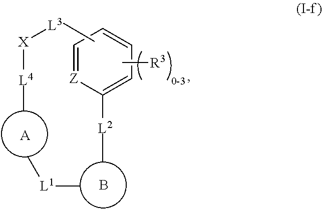

or a pharmaceutically acceptable salt thereof, wherein Z is CH or N.

In some embodiments, the compound is a compound of Formula (I-g):

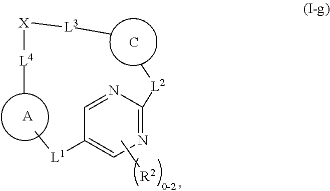

or a pharmaceutically acceptable salt thereof.

In some embodiments, the compound is a compound of Formula (I-h):

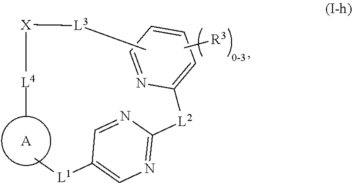

or a pharmaceutically acceptable salt thereof.

In some embodiments, the compound of Formula (I) is selected from the group consisting of the compounds in Table 1, or a pharmaceutically acceptable salt thereof.

TABLE 1
Exemplary Compounds of Formula (I)

| Compound | |
|---|---|
| No. | Structure |
| 1 | 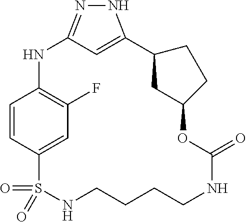 [SMILES: O=C1NCCCCNS(=O)(=O)c2ccc(c(F)c2)Nc2cc([nH]n2)[C@H]2CC[C@H](C2)O1] |
| 2 | 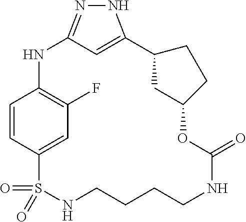 [SMILES: O=C1NCCCCNS(=O)(=O)c2ccc(c(F)c2)Nc2cc([nH]n2)[C@@H]2CC[C@@H](C2)O1] |
| 3 | 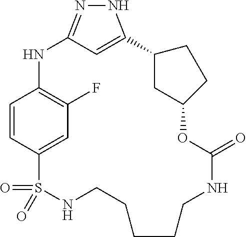 [SMILES: O=C1NCCCCCNS(=O)(=O)c2ccc(c(F)c2)Nc2cc([nH]n2)[C@@H]2CC[C@@H](C2)O1] |
| 4 | 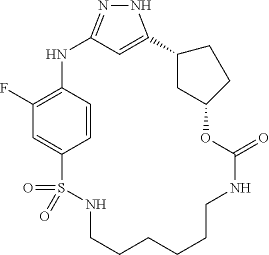 [SMILES: O=C1NCCCCCCNS(=O)(=O)c2ccc(c(F)c2)Nc2cc([nH]n2)[C@@H]2CC[C@@H](C2)O1] |
| 5 | 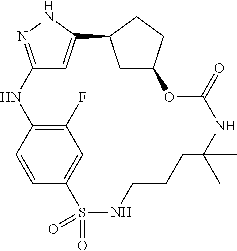 [SMILES: CC1(C)CCCNS(=O)(=O)c2ccc(c(F)c2)Nc2cc([nH]n2)[C@H]2CC[C@H](C2)OC(=O)N1] |
| 6 | 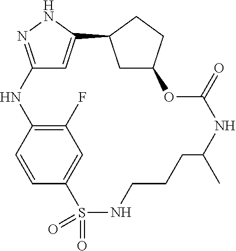 [SMILES: CC1CCCNS(=O)(=O)c2ccc(c(F)c2)Nc2cc([nH]n2)[C@H]2CC[C@H](C2)OC(=O)N1] |
| 7 | 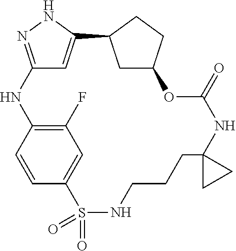 [SMILES: O=C1NC2(CCCNS(=O)(=O)c3ccc(c(F)c3)Nc3cc([nH]n3)[C@H]3CC[C@H](C3)O1)CC2] |
| 8 | 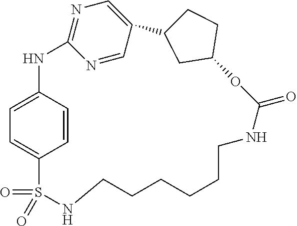 |
| 9 | 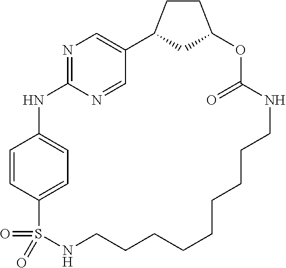 [SMILES: O=C1NCCCCCCCCCNS(=O)(=O)c2ccc(cc2)Nc2ncc(cn2)[C@@H]2CC[C@@H](C2)O1] |
| 10 | 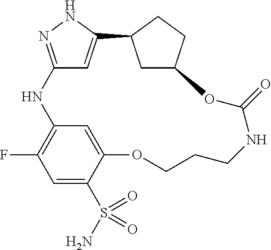 [SMILES: NS(=O)(=O)c1cc(F)c2cc1OCCCNC(=O)O[C@@H]1CC[C@@H](C1)c1cc(n[nH]1)N2] |
| 11 | 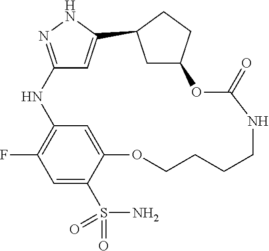 [SMILES: NS(=O)(=O)c1cc(F)c2cc1OCCCCNC(=O)O[C@@H]1CC[C@@H](C1)c1cc(n[nH]1)N2] |
| 12 | 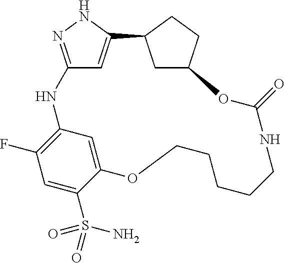 [SMILES: NS(=O)(=O)c1cc(F)c2cc1OCCCCCNC(=O)O[C@@H]1CC[C@@H](C1)c1cc(n[nH]1)N2] |
| 13 | 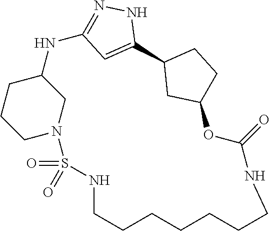 [SMILES: O=C1NCCCCCCCNS(=O)(=O)N2CCCC(C2)Nc2cc([nH]n2)[C@H]2CC[C@H](C2)O1] |
| 14 | 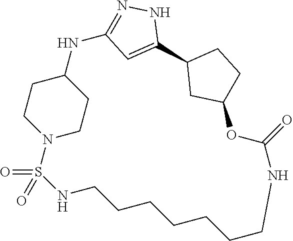 [SMILES: O=C1NCCCCCCCNS(=O)(=O)N2CCC(CC2)Nc2cc([nH]n2)[C@H]2CC[C@H](C2)O1] |
| 15 | 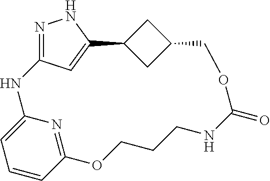 [SMILES: O=C1NCCCOc2cccc(n2)Nc2cc([nH]n2)[C@H]2C[C@H](CO1)C2] |
| 16 | 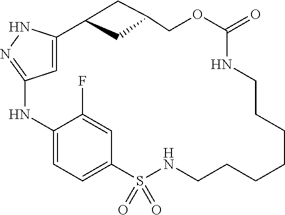 [SMILES: O=C1NCCCCCCCNS(=O)(=O)c2ccc(c(F)c2)Nc2cc([nH]n2)[C@H]2C[C@H](CO1)C2] |
| 17 | 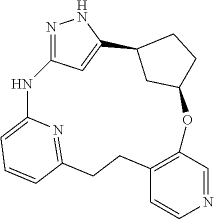 [SMILES: c1cc2nc(c1)Nc1cc([nH]n1)[C@H]1CC[C@H](C1)Oc1cnccc1CC2] |
| 18 | 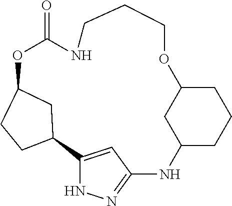 [SMILES: O=C1NCCCOC2CCCC(C2)Nc2cc([nH]n2)[C@H]2CC[C@H](C2)O1] |
| 19 | 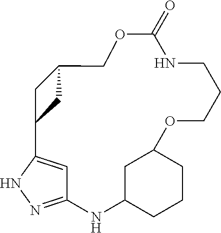 [SMILES: O=C1NCCCOC2CCCC(C2)Nc2cc([nH]n2)[C@H]2C[C@H](CO1)C2] |
| 20 | 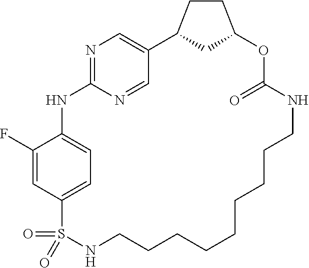 [SMILES: O=C1NCCCCCCCCCNS(=O)(=O)c2ccc(c(F)c2)Nc2ncc(cn2)[C@@H]2CC[C@@H](C2)O1] |
| 21 | 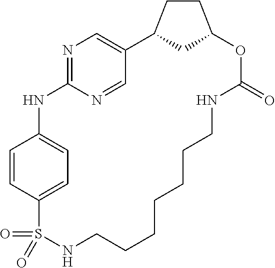 [SMILES: O=C1NCCCCCCCNS(=O)(=O)c2ccc(cc2)Nc2ncc(cn2)[C@@H]2CC[C@@H](C2)O1] |
| 22 | 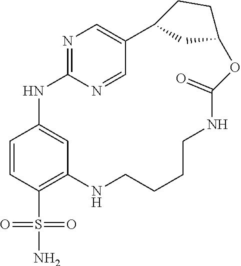 [SMILES: NS(=O)(=O)c1ccc2cc1NCCCCNC(=O)O[C@H]1CC[C@H](C1)c1cnc(nc1)N2] |
| 23 | 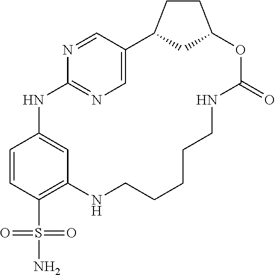 [SMILES: NS(=O)(=O)c1ccc2cc1NCCCCCNC(=O)O[C@H]1CC[C@H](C1)c1cnc(nc1)N2] |
| 24 | 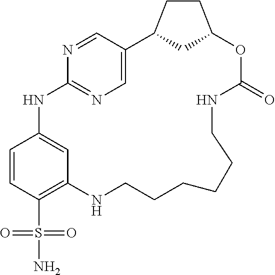 [SMILES: NS(=O)(=O)c1ccc2cc1NCCCCCCNC(=O)O[C@H]1CC[C@H](C1)c1cnc(nc1)N2] |
| 25 | 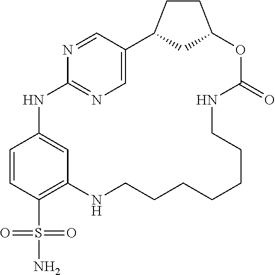 [SMILES: NS(=O)(=O)c1ccc2cc1NCCCCCCCNC(=O)O[C@H]1CC[C@H](C1)c1cnc(nc1)N2] |
| 26 | 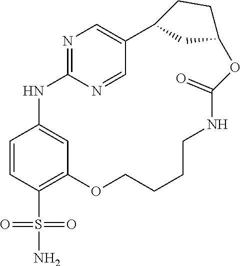 [SMILES: NS(=O)(=O)c1ccc2cc1OCCCCNC(=O)O[C@H]1CC[C@H](C1)c1cnc(nc1)N2] |
| 27 | 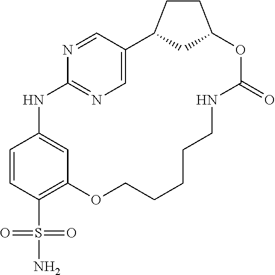 [SMILES: NS(=O)(=O)c1ccc2cc1OCCCCCNC(=O)O[C@H]1CC[C@H](C1)c1cnc(nc1)N2] |
| 28 | 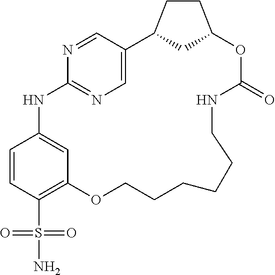 [SMILES: NS(=O)(=O)c1ccc2cc1OCCCCCCNC(=O)O[C@H]1CC[C@H](C1)c1cnc(nc1)N2] |
| 29 | 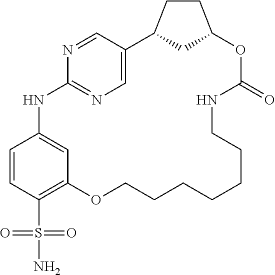 [SMILES: NS(=O)(=O)c1ccc2cc1OCCCCCCCNC(=O)O[C@H]1CC[C@H](C1)c1cnc(nc1)N2] |
| 30 | 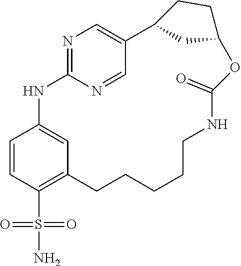 [SMILES: NS(=O)(=O)c1ccc2cc1CCCCCNC(=O)O[C@H]1CC[C@H](C1)c1cnc(nc1)N2] |
| 31 | 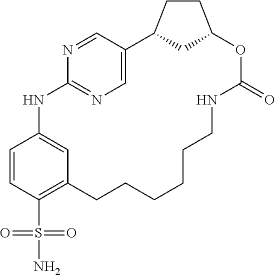 [SMILES: NS(=O)(=O)c1ccc2cc1CCCCCCNC(=O)O[C@H]1CC[C@H](C1)c1cnc(nc1)N2] |
| 32 | 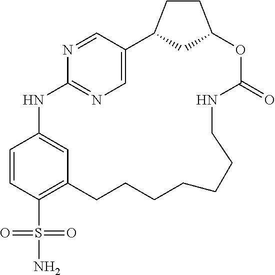 [SMILES: NS(=O)(=O)c1ccc2cc1CCCCCCCNC(=O)O[C@H]1CC[C@H](C1)c1cnc(nc1)N2] |
| 33 | 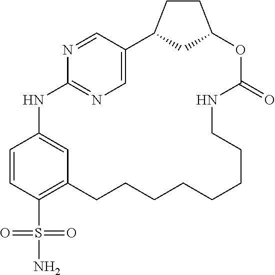 [SMILES: NS(=O)(=O)c1ccc2cc1CCCCCCCCNC(=O)O[C@H]1CC[C@H](C1)c1cnc(nc1)N2] |
| 34 | 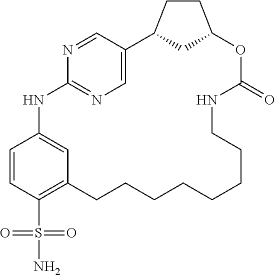 [SMILES: NS(=O)(=O)c1ccc2cc1CCCCCCCCNC(=O)O[C@H]1CC[C@H](C1)c1cnc(nc1)N2] |
| 35 | 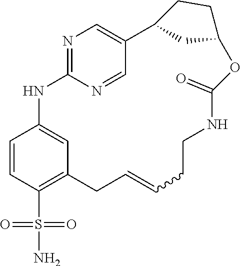 [SMILES: NS(=O)(=O)c1ccc2cc1CC=CCCNC(=O)O[C@H]1CC[C@H](C1)c1cnc(nc1)N2] |
| 36 | 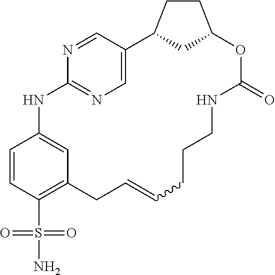 [SMILES: NS(=O)(=O)c1ccc2cc1CC=CCCCNC(=O)O[C@H]1CC[C@H](C1)c1cnc(nc1)N2] |
| 37 | 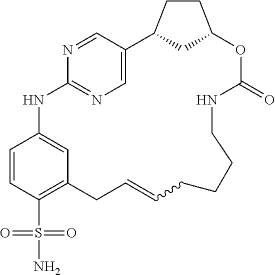 [SMILES: NS(=O)(=O)c1ccc2cc1CC=CCCCCNC(=O)O[C@H]1CC[C@H](C1)c1cnc(nc1)N2] |
| 38 | 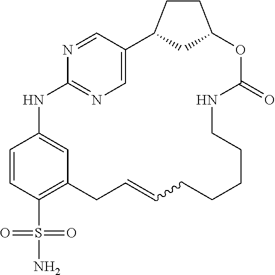 [SMILES: NS(=O)(=O)c1ccc2cc1CC=CCCCCCNC(=O)O[C@H]1CC[C@H](C1)c1cnc(nc1)N2] |
| 39 | 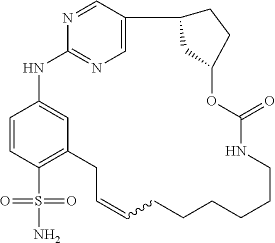 [SMILES: NS(=O)(=O)c1ccc2cc1CC=CCCCCCCNC(=O)O[C@H]1CC[C@H](C1)c1cnc(nc1)N2] |
| 40 | 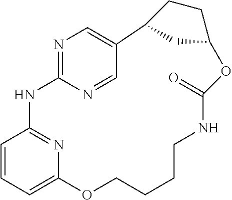 [SMILES: O=C1NCCCCOc2cccc(n2)Nc2ncc(cn2)[C@@H]2CC[C@@H](C2)O1] |
| 41 | 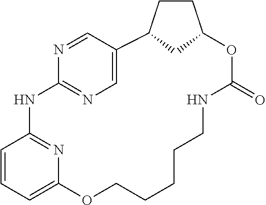 [SMILES: O=C1NCCCCCOc2cccc(n2)Nc2ncc(cn2)[C@@H]2CC[C@@H](C2)O1] |
| 42 | 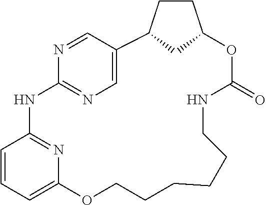 [SMILES: O=C1NCCCCCCOc2cccc(n2)Nc2ncc(cn2)[C@@H]2CC[C@@H](C2)O1] |
| 43 | 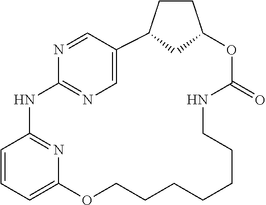 [SMILES: O=C1NCCCCCCCOc2cccc(n2)Nc2ncc(cn2)[C@@H]2CC[C@@H](C2)O1] |
| 44 | 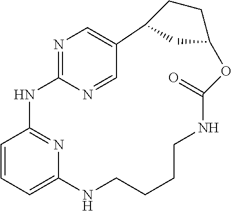 [SMILES: O=C1NCCCCNc2cccc(n2)Nc2ncc(cn2)[C@@H]2CC[C@@H](C2)O1] |
| 45 | 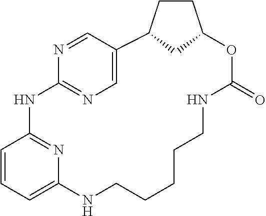 [SMILES: O=C1NCCCCCNc2cccc(n2)Nc2ncc(cn2)[C@@H]2CC[C@@H](C2)O1] |
| 46 | 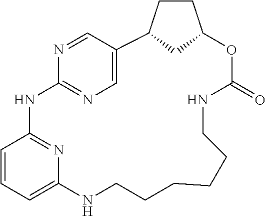 [SMILES: O=C1NCCCCCCNc2cccc(n2)Nc2ncc(cn2)[C@@H]2CC[C@@H](C2)O1] |
| 47 | 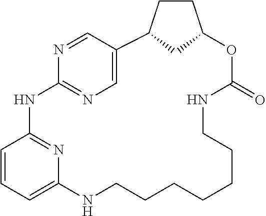 [SMILES: O=C1NCCCCCCCNc2cccc(n2)Nc2ncc(cn2)[C@@H]2CC[C@@H](C2)O1] |
| 48 | 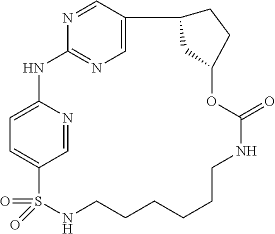 [SMILES: O=C1NCCCCCCNS(=O)(=O)c2ccc(nc2)Nc2ncc(cn2)[C@@H]2CC[C@@H](C2)O1] |
| 49 | 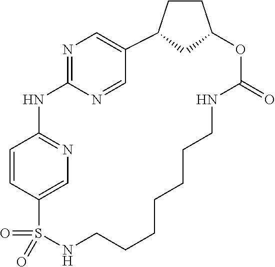 [SMILES: O=C1NCCCCCCCNS(=O)(=O)c2ccc(nc2)Nc2ncc(cn2)[C@@H]2CC[C@@H](C2)O1] |
| 50 | 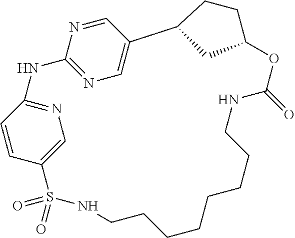 [SMILES: O=C1NCCCCCCCCNS(=O)(=O)c2ccc(nc2)Nc2ncc(cn2)[C@@H]2CC[C@@H](C2)O1] |
| 51 | 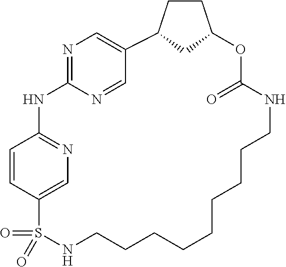 [SMILES: O=C1NCCCCCCCCCNS(=O)(=O)c2ccc(nc2)Nc2ncc(cn2)[C@@H]2CC[C@@H](C2)O1] |
| 52 | 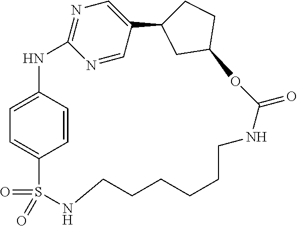 [SMILES: O=C1NCCCCCCNS(=O)(=O)c2ccc(cc2)Nc2ncc(cn2)[C@H]2CC[C@H](C2)O1] |
| 53 | 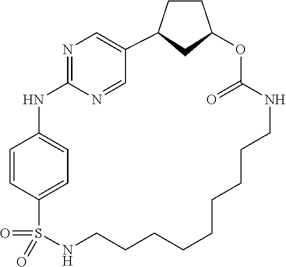 [SMILES: O=C1NCCCCCCCCCNS(=O)(=O)c2ccc(cc2)Nc2ncc(cn2)[C@H]2CC[C@H](C2)O1] |
| 54 | 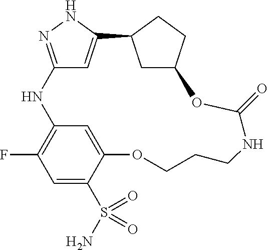 [SMILES: NS(=O)(=O)c1cc(F)c2cc1OCCCNC(=O)O[C@@H]1CC[C@@H](C1)c1cc(n[nH]1)N2] |
| 55 | 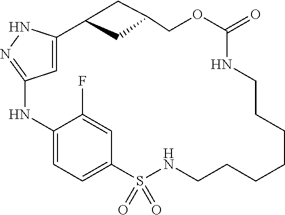 [SMILES: O=C1NCCCCCCCNS(=O)(=O)c2ccc(c(F)c2)Nc2cc([nH]n2)[C@H]2C[C@H](CO1)C2] |
| 56 | 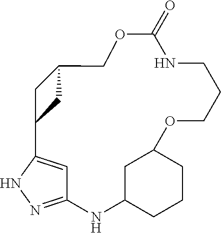 [SMILES: O=C1NCCCOC2CCCC(C2)Nc2cc([nH]n2)[C@H]2C[C@H](CO1)C2] |
| 57 | 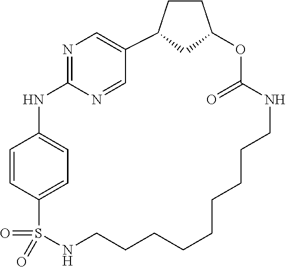 [SMILES: O=C1NCCCCCCCCCNS(=O)(=O)c2ccc(cc2)Nc2ncc(cn2)[C@@H]2CC[C@@H](C2)O1] |
| 58 | 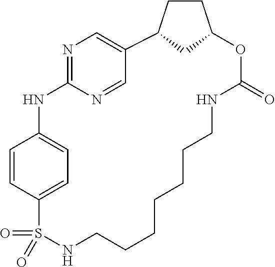 [SMILES: O=C1NCCCCCCCNS(=O)(=O)c2ccc(cc2)Nc2ncc(cn2)[C@@H]2CC[C@@H](C2)O1] |
| 59 | 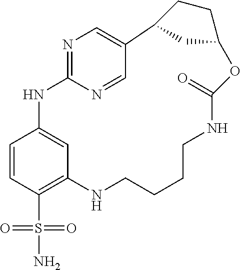 [SMILES: NS(=O)(=O)c1ccc2cc1NCCCCNC(=O)O[C@H]1CC[C@H](C1)c1cnc(nc1)N2] |
| 60 | 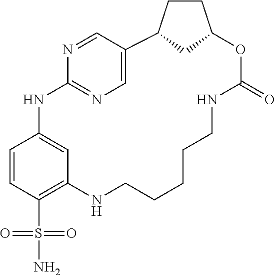 [SMILES: NS(=O)(=O)c1ccc2cc1NCCCCCNC(=O)O[C@H]1CC[C@H](C1)c1cnc(nc1)N2] |
| 61 | 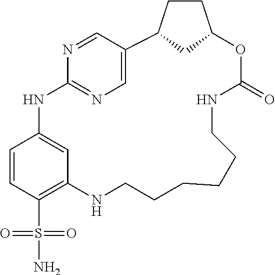 [SMILES: NS(=O)(=O)c1ccc2cc1NCCCCCCNC(=O)O[C@H]1CC[C@H](C1)c1cnc(nc1)N2] |
| 62 | 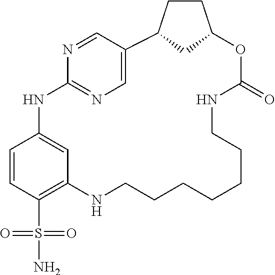 [SMILES: NS(=O)(=O)c1ccc2cc1NCCCCCCCNC(=O)O[C@H]1CC[C@H](C1)c1cnc(nc1)N2] |
| 63 | 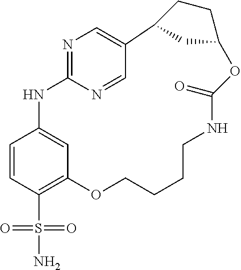 [SMILES: NS(=O)(=O)c1ccc2cc1OCCCCNC(=O)O[C@H]1CC[C@H](C1)c1cnc(nc1)N2] |
| 64 | 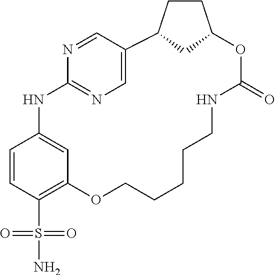 [SMILES: NS(=O)(=O)c1ccc2cc1OCCCCCNC(=O)O[C@H]1CC[C@H](C1)c1cnc(nc1)N2] |
| 65 | 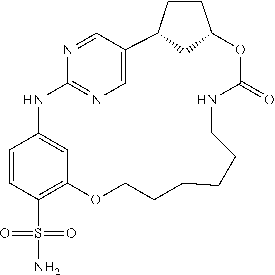 [SMILES: NS(=O)(=O)c1ccc2cc1OCCCCCCNC(=O)O[C@H]1CC[C@H](C1)c1cnc(nc1)N2] |
| 66 | 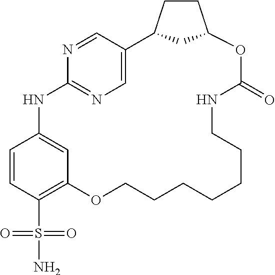 [SMILES: NS(=O)(=O)c1ccc2cc1OCCCCCCCNC(=O)O[C@H]1CC[C@H](C1)c1cnc(nc1)N2] |
| 67 | 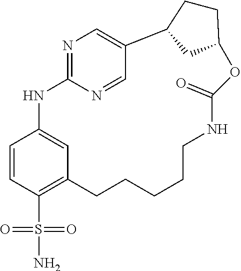 [SMILES: NS(=O)(=O)c1ccc2cc1CCCCCNC(=O)O[C@H]1CC[C@H](C1)c1cnc(nc1)N2] |
| 68 | 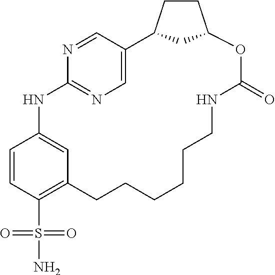 [SMILES: NS(=O)(=O)c1ccc2cc1CCCCCCNC(=O)O[C@H]1CC[C@H](C1)c1cnc(nc1)N2] |
| 69 | 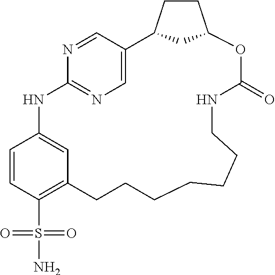 [SMILES: NS(=O)(=O)c1ccc2cc1CCCCCCCNC(=O)O[C@H]1CC[C@H](C1)c1cnc(nc1)N2] |
| 70 | 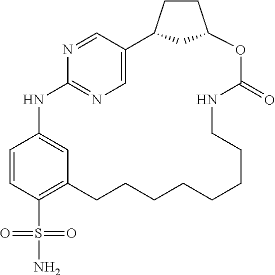 [SMILES: NS(=O)(=O)c1ccc2cc1CCCCCCCCNC(=O)O[C@H]1CC[C@H](C1)c1cnc(nc1)N2] |
| 71 | 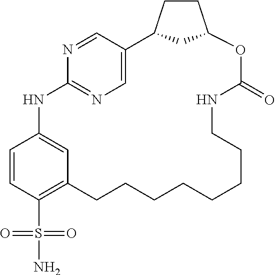 [SMILES: NS(=O)(=O)c1ccc2cc1CCCCCCCCNC(=O)O[C@H]1CC[C@H](C1)c1cnc(nc1)N2] |
| 72 | 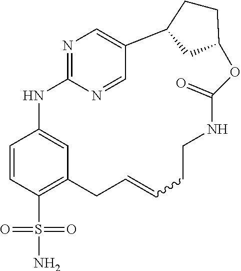 [SMILES: NS(=O)(=O)c1ccc2cc1CC=CCCNC(=O)O[C@H]1CC[C@H](C1)c1cnc(nc1)N2] |
| 73 | 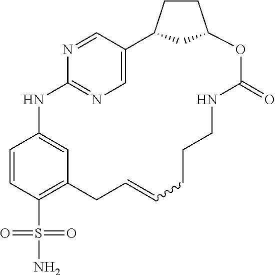 [SMILES: NS(=O)(=O)c1ccc2cc1CC=CCCCNC(=O)O[C@H]1CC[C@H](C1)c1cnc(nc1)N2] |
| 74 | 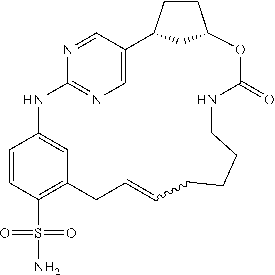 [SMILES: NS(=O)(=O)c1ccc2cc1CC=CCCCCNC(=O)O[C@H]1CC[C@H](C1)c1cnc(nc1)N2] |
| 75 | 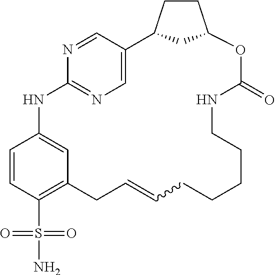 [SMILES: NS(=O)(=O)c1ccc2cc1CC=CCCCCCNC(=O)O[C@H]1CC[C@H](C1)c1cnc(nc1)N2] |
| 76 | 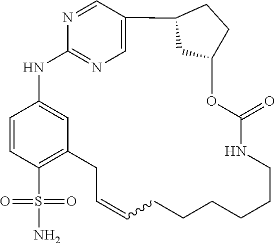 [SMILES: NS(=O)(=O)c1ccc2cc1CC=CCCCCCCNC(=O)O[C@H]1CC[C@H](C1)c1cnc(nc1)N2] |
| 77 | 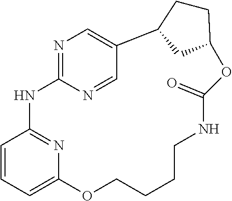 [SMILES: O=C1NCCCCOc2cccc(n2)Nc2ncc(cn2)[C@@H]2CC[C@@H](C2)O1] |
| 78 | 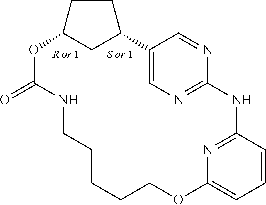 [SMILES: O=C1NCCCCCOc2cccc(n2)Nc2ncc(cn2)[C@H]2CC[C@H](C2)O1] |
| 79 | 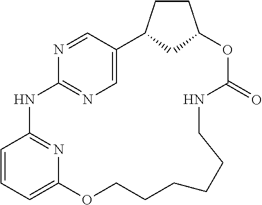 [SMILES: O=C1NCCCCCCOc2cccc(n2)Nc2ncc(cn2)[C@@H]2CC[C@@H](C2)O1] |
| 80 | 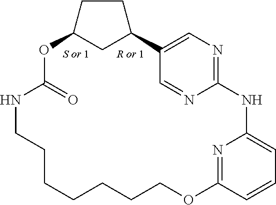 [SMILES: O=C1NCCCCCCCOc2cccc(n2)Nc2ncc(cn2)[C@@H]2CC[C@@H](C2)O1] |
| 81 | 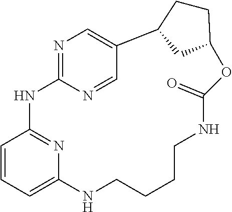 [SMILES: O=C1NCCCCNc2cccc(n2)Nc2ncc(cn2)[C@@H]2CC[C@@H](C2)O1] |
| 82 | 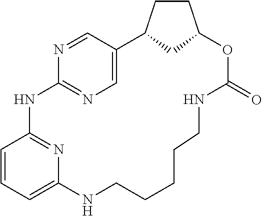 [SMILES: O=C1NCCCCCNc2cccc(n2)Nc2ncc(cn2)[C@@H]2CC[C@@H](C2)O1] |
| 83 | 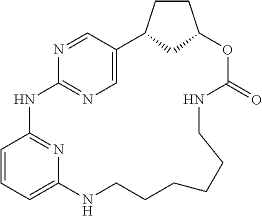 [SMILES: O=C1NCCCCCCNc2cccc(n2)Nc2ncc(cn2)[C@@H]2CC[C@@H](C2)O1] |
| 84 | 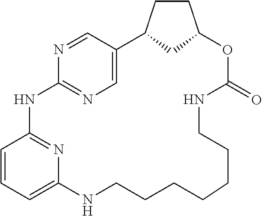 [SMILES: O=C1NCCCCCCCNc2cccc(n2)Nc2ncc(cn2)[C@@H]2CC[C@@H](C2)O1] |
| 85 | 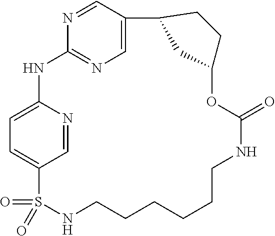 [SMILES: O=C1NCCCCCCNS(=O)(=O)c2ccc(nc2)Nc2ncc(cn2)[C@@H]2CC[C@@H](C2)O1] |
| 86 | 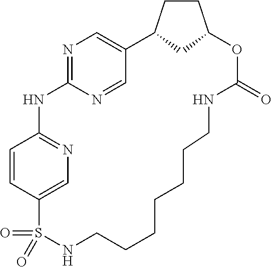 [SMILES: O=C1NCCCCCCCNS(=O)(=O)c2ccc(nc2)Nc2ncc(cn2)[C@@H]2CC[C@@H](C2)O1] |
| 87 | 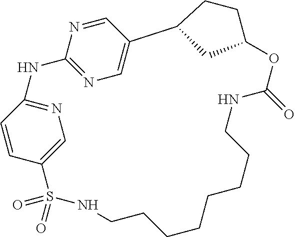 [SMILES: O=C1NCCCCCCCCNS(=O)(=O)c2ccc(nc2)Nc2ncc(cn2)[C@@H]2CC[C@@H](C2)O1] |
| 88 | 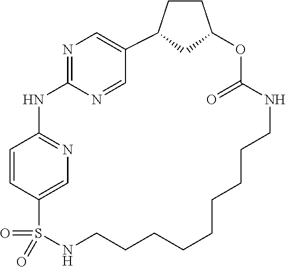 [SMILES: O=C1NCCCCCCCCCNS(=O)(=O)c2ccc(nc2)Nc2ncc(cn2)[C@@H]2CC[C@@H](C2)O1] |
| 89 | 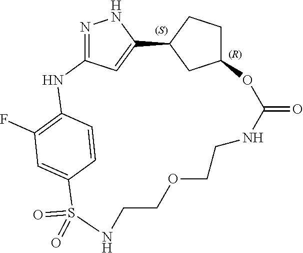 |
| 90 | 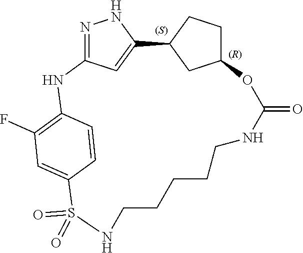 |
| 91 | 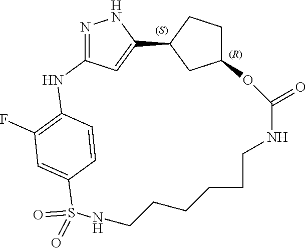 |
| 92 | 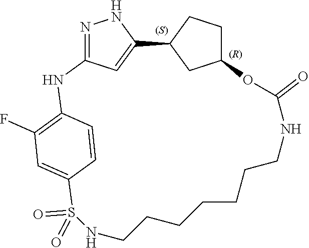 |
| 93 | 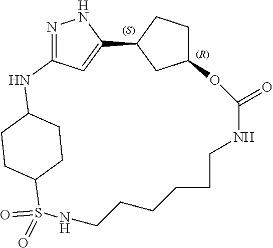 |
| 94 | 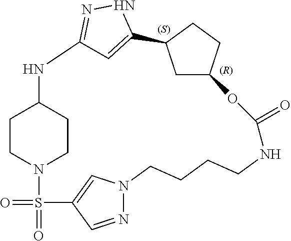 |
| 95 | 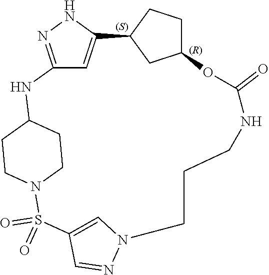 |
| 96 | 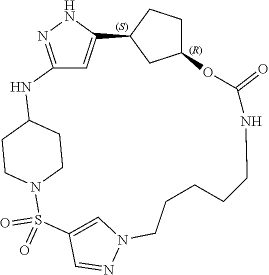 |
| 97 | 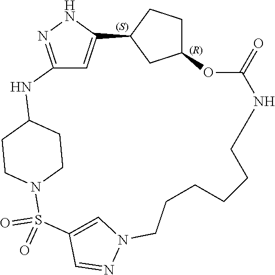 |
| 98 | 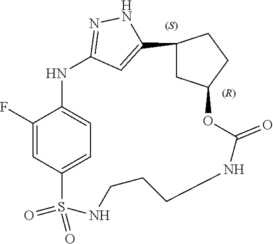 |
| 99 | 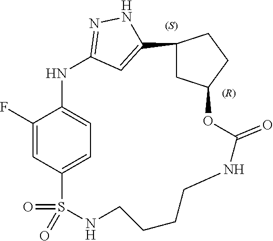 |
| 100 | 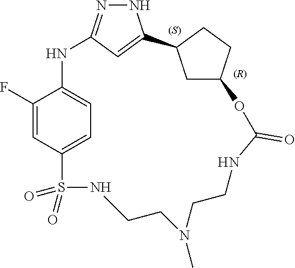 |
| 101 | 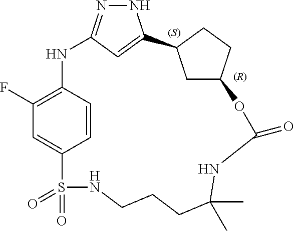 |
| 102 | 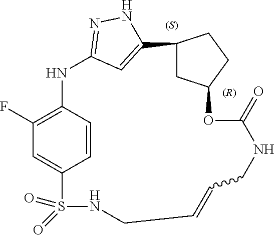 |
| 103 | 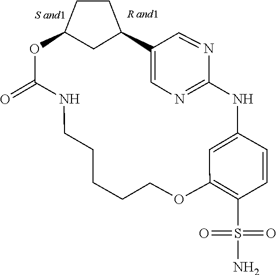 [SMILES: NS(=O)(=O)c1ccc2cc1OCCCCCNC(=O)O[C@H]1CC[C@H](C1)c1cnc(nc1)N2] |
| 104 | 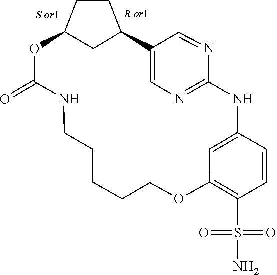 [SMILES: NS(=O)(=O)c1ccc2cc1OCCCCCNC(=O)O[C@H]1CC[C@H](C1)c1cnc(nc1)N2] |
| 105 | 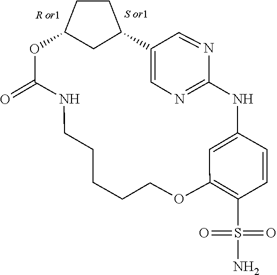 [SMILES: NS(=O)(=O)c1ccc2cc1OCCCCCNC(=O)O[C@@H]1CC[C@@H](C1)c1cnc(nc1)N2] |
| 106 | 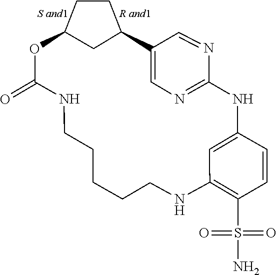 [SMILES: NS(=O)(=O)c1ccc2cc1NCCCCCNC(=O)O[C@H]1CC[C@H](C1)c1cnc(nc1)N2] |
| 107 | 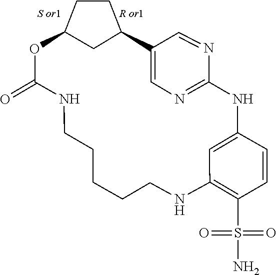 [SMILES: NS(=O)(=O)c1ccc2cc1NCCCCCNC(=O)O[C@H]1CC[C@H](C1)c1cnc(nc1)N2] |
| 108 | 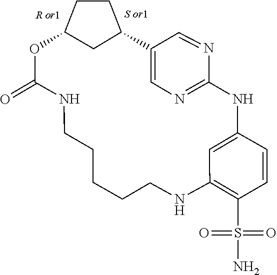 [SMILES: NS(=O)(=O)c1ccc2cc1NCCCCCNC(=O)O[C@@H]1CC[C@@H](C1)c1cnc(nc1)N2] |
| 109 | 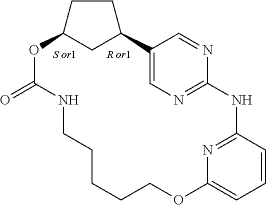 [SMILES: O=C1NCCCCCOc2cccc(n2)Nc2ncc(cn2)[C@@H]2CC[C@@H](C2)O1] |
| 110 | 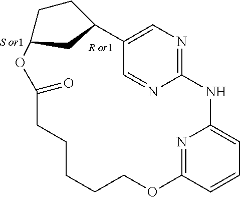 [SMILES: O=C1CCCCCOc2cccc(n2)Nc2ncc(cn2)[C@@H]2CC[C@@H](C2)O1] |
| 111 | 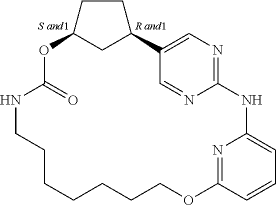 [SMILES: O=C1NCCCCCCCOc2cccc(n2)Nc2ncc(cn2)[C@@H]2CC[C@@H](C2)O1] |
| 112 | 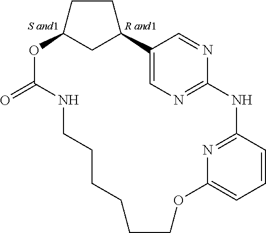 [SMILES: O=C1NCCCCCCOc2cccc(n2)Nc2ncc(cn2)[C@@H]2CC[C@@H](C2)O1] |
| 113 | 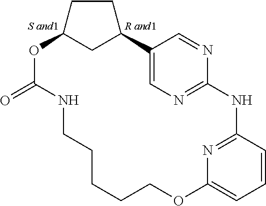 [SMILES: O=C1NCCCCCOc2cccc(n2)Nc2ncc(cn2)[C@@H]2CC[C@@H](C2)O1] |
| 114 | 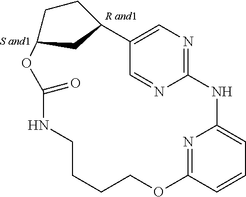 [SMILES: O=C1NCCCCOc2cccc(n2)Nc2ncc(cn2)[C@@H]2CC[C@@H](C2)O1] |
| 115 | 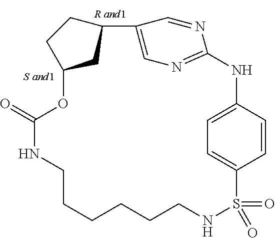 [SMILES: O=C1NCCCCCCNS(=O)(=O)c2ccc(cc2)Nc2ncc(cn2)[C@@H]2CC[C@@H](C2)O1] |

### Methods of Treatment

Some embodiments provide a method of treating cancer (e.g., a CDK2-associated cancer) in a subject in need thereof, comprising administering a therapeutically effective amount of a compound of Formula (I), or a pharmaceutically acceptable salt thereof or a pharmaceutical composition thereof, to the subject. For example, provided herein are methods for treating a CDK2-associated cancer in a subject in need thereof, comprising a) detecting a dysregulation of a CDK2 gene, a CDK2 protein, or the expression or activity or level of any of the same in a sample from the subject; and b) administering a therapeutically effective amount of a compound of Formula (I), or a pharmaceutically acceptable salt thereof.

In some embodiments, the subject has been identified or diagnosed as having a cancer with a dysregulation of a CDK2 gene, a CDK2 protein, or expression or activity, or level of any of the same (a CDK2-associated-associated cancer) (e.g., as determined using a regulatory agency-approved, e.g., FDA-approved, assay or kit). In some embodiments, the subject has been identified or diagnosed as having a cancer with a dysregulation of a cyclin A2 gene, a cyclin A2 protein, or expression or activity, or level of any of the same (a CDK2-associated-associated cancer) (e.g., as determined using a regulatory agency-approved, e.g., FDA-approved, assay or kit). In some embodiments, the subject has been identified or diagnosed as having a cancer with a dysregulation of a cyclin E1 gene, a cyclin E1 protein, or expression or activity, or level of any of the same (a CDK2-associated-associated cancer) (e.g., as determined using a regulatory agency-approved, e.g., FDA-approved, assay or kit). In some embodiments, the subject has been identified or diagnosed as having a cancer with a dysregulation of a cyclin E2 gene, a cyclin E2 protein, or expression or activity, or level of any of the same (a CDK2-associated-associated cancer) (e.g., as determined using a regulatory agency-approved, e.g., FDA-approved, assay or kit). In some embodiments, the subject has been identified or diagnosed as having a cancer with a dysregulation of a CDK2 gene, a CDK2 protein, a cyclin A2 gene, a cyclin A2 protein, a cyclin E1 gene, a cyclin E1 protein, a cyclin E2 gene, a cyclin E2 protein, or expression or activity, or level of any of the same (or any combination thereof).

In some embodiments, the subject has a tumor that is positive for a dysregulation of a CDK2 gene, a CDK2 protein, or expression or activity, or level of any of the same (e.g., as determined using a regulatory agency-approved, e.g., FDA-approved, assay or kit). The subject can be a subject with a tumor(s) that is positive for a dysregulation of a CDK2 gene, a CDK2 protein, or expression or activity, or level of any of the same (e.g., identified as positive using a regulatory agency-approved, e.g., FDA-approved, assay or kit). The subject can be a subject whose tumors have a dysregulation of a CDK2 gene, a CDK2 protein, or expression or activity, or level of any of the same (e.g., where the tumor is identified as such using a regulatory agency-approved, e.g., FDA-approved, kit or assay).

In some embodiments, the subject has a tumor that is positive for a dysregulation of a cyclin A2 gene, a cyclin A2 protein, or expression or activity, or level of any of the same (e.g., as determined using a regulatory agency-approved, e.g., FDA-approved, assay or kit). The subject can be a subject with a tumor(s) that is positive for a dysregulation of a cyclin A2 gene, a cyclin A2 protein, or expression or activity, or level of any of the same (e.g., identified as positive using a regulatory agency-approved, e.g., FDA-approved, assay or kit). The subject can be a subject whose tumors have a dysregulation of a cyclin A2 gene, a cyclin A2 protein, or expression or activity, or level of any of the same (e.g., where the tumor is identified as such using a regulatory agency-approved, e.g., FDA-approved, kit or assay).

In some embodiments, the subject has a tumor that is positive for a dysregulation of a cyclin E1 gene, a cyclin E1 protein, or expression or activity, or level of any of the same (e.g., as determined using a regulatory agency-approved, e.g., FDA-approved, assay or kit). The subject can be a subject with a tumor(s) that is positive for a dysregulation of a cyclin E1 gene, a cyclin E1 protein, or expression or activity, or level of any of the same (e.g., identified as positive using a regulatory agency-approved, e.g., FDA-approved, assay or kit). The subject can be a subject whose tumors have a dysregulation of a cyclin E1 gene, a cyclin E1 protein, or expression or activity, or level of any of the same (e.g., where the tumor is identified as such using a regulatory agency-approved, e.g., FDA-approved, kit or assay).

In some embodiments, the subject has a tumor that is positive for a dysregulation of a cyclin E2 gene, a cyclin E2 protein, or expression or activity, or level of any of the same (e.g., as determined using a regulatory agency-approved, e.g., FDA-approved, assay or kit). The subject can be a subject with a tumor(s) that is positive for a dysregulation of a cyclin E2 gene, a cyclin E2 protein, or expression or activity, or level of any of the same (e.g., identified as positive using a regulatory agency-approved, e.g., FDA-approved, assay or kit). The subject can be a subject whose tumors have a dysregulation of a cyclin E2 gene, a cyclin E2 protein, or expression or activity, or level of any of the same (e.g., where the tumor is identified as such using a regulatory agency-approved, e.g., FDA-approved, kit or assay).

In some embodiments, the subject has a tumor that is positive for a dysregulation of a CDK2 gene, a CDK2 protein, a cyclin A2 gene, a cyclin A2 protein, a cyclin E1 gene, a cyclin E1 protein, a cyclin E2 gene, a cyclin E2 protein, or expression or activity, or level of any of the same (or any combination thereof).

In some embodiments, a dysregulation can be a dysregulation that results in aberrant activation of a gene, protein, or expression or activity or level of any of the same. Activation can be through any appropriate mechanism, including, but not limited to, gene amplification, activating mutation, activating translocation, transcriptional activation, epigenetic alteration, and/or overexpression of the protein product of the oncogene. In some embodiments, a dysregulation can be a dysregulation that results in aberrant inactivation of a gene, protein, or expression or activity or level of any of the same. Inactivation can be through any appropriate mechanism, including, but not limited to, gene deletion, inactivating mutation, inactivating translocation, transcriptional silencing, epigenetic alteration, and degradation of mRNA and/or protein products of the gene. Typically, as used herein, a dysregulation results in aberrations in the cell cycle.

In some embodiments, the subject is suspected of having a CDK2-associated-associated cancer.

In some embodiments, the subject has a clinical record indicating that the subject has a tumor that has a dysregulation of a CDK2 gene, a CDK2 protein, or expression or activity, or level of any of the same (and optionally the clinical record indicates that the subject should be treated with any of the compositions provided herein). In some embodiments, the subject has a clinical record indicating that the subject has a tumor that has a dysregulation of a cyclin A2 gene, a cyclin A2 protein, or expression or activity, or level of any of the same (and optionally the clinical record indicates that the subject should be treated with any of the compositions provided herein). In some embodiments, the subject has a clinical record indicating that the subject has a tumor that has a dysregulation of a cyclin E1 gene, a cyclin E1 protein, or expression or activity, or level of any of the same (and optionally the clinical record indicates that the subject should be treated with any of the compositions provided herein). In some embodiments, the subject has a clinical record indicating that the subject has a tumor that has a dysregulation of a cyclin E2 gene, a cyclin E2 protein, or expression or activity, or level of any of the same (and optionally the clinical record indicates that the subject should be treated with any of the compositions provided herein). In some embodiments, the subject has a clinical record indicating that the subject has a tumor that has a dysregulation of a CDK2 gene, a CDK2 protein, a cyclin A2 gene, a cyclin A2 protein, a cyclin E1 gene, a cyclin E1 protein, a cyclin E2 gene, a cyclin E2 protein, or expression or activity, or level of any of the same (or any combination thereof).

In some embodiments, the subject has been identified or diagnosed as having a cancer that, based on histological examination, is determined to be associated with a dysregulation of a CDK2 gene, a CDK2 protein, or expression or activity, or level of any of the same (a CDK2-associated-associated cancer). In some embodiments, the subject has been identified or diagnosed as having a cancer that, based on histological examination, is determined to be associated with a dysregulation of a cyclin A2 gene, a cyclin A2 protein, or expression or activity, or level of any of the same (a CDK2-associated-associated cancer). In some embodiments, the subject has been identified or diagnosed as having a cancer that, based on histological examination, is determined to be associated with a dysregulation of a cyclin E1 gene, a cyclin E1 protein, or expression or activity, or level of any of the same (a CDK2-associated-associated cancer). In some embodiments, the subject has been identified or diagnosed as having a cancer that, based on histological examination, is determined to be associated with a dysregulation of a cyclin E2 gene, a cyclin E2 protein, or expression or activity, or level of any of the same (a CDK2-associated-associated cancer). In some embodiments, the subject has been identified or diagnosed as having a cancer that, based on histological examination, is determined to be associated with a dysregulation of a CDK2 gene, a CDK2 protein, a cyclin A2 gene, a cyclin A2 protein, a cyclin E1 gene, a cyclin E1 protein, a cyclin E2 gene, a cyclin E2 protein, or expression or activity, or level of any of the same (or any combination thereof).

In some embodiments, the subject has a clinical record indicating that the subject has a tumor resistant to one or more previous therapies, for example, resistance to CDK4/CDK6 inhibition. In some embodiments, the subject has a cancer resistant to one or more previous therapies, for example, resistance to CDK4/CDK6 inhibition.

In some embodiments, the subject has a tumor resistant to one or more previous therapies, for example, resistance to CDK4/CDK6 inhibition. In some embodiments, the subject has a tumor that is suspected of being resistant to one or more previous therapies, for example, resistance to CDK4/CDK6 inhibition.

In some embodiments of any of the methods or uses described herein, the cancer (e.g., CDK2-associated cancer) is a solid tumor. In some embodiments of any of the methods or uses described herein, the cancer (e.g., CDK2-associated cancer) is colorectal cancer, lung cancer (including small cell lung carcinoma, non-small cell lung carcinoma, squamous cell carcinoma, and adenocarcinoma), thyroid cancer, breast cancer, ovarian cancer, bladder cancer, uterine cancer, prostate cancer, esophageal cancer, head and neck cancer, kidney cancer (including RCC), liver cancer (including HCC), pancreatic cancer, or stomach (i.e., gastric) cancer.

In some embodiments of any of the methods or uses described herein, the cancer (e.g., CDK2-associated cancer) is selected from the group consisting of breast cancer, ovarian cancer, bladder cancer, uterine cancer, prostate cancer, lung cancer, esophageal cancer, liver cancer, pancreatic cancer and stomach cancer.

In some embodiments of any of the methods or uses described herein, the cancer (e.g., CDK2-associated cancer) is selected from the group consisting of breast cancer, ovarian cancer, and colorectal cancer.

In some embodiments of any of the methods or uses described herein, the cancer (e.g., CDK2-associated cancer) is colorectal cancer.

In some embodiments of any of the methods or uses described herein, the cancer (e.g., CDK2-associated cancer) is selected from the group consisting of breast cancer and ovarian cancer.

In some embodiments of any of the methods or uses described herein, the cancer (e.g., CDK2-associated cancer) is ovarian cancer.

In some embodiments of any of the methods or uses described herein, the cancer (e.g., CDK2-associated cancer) is breast cancer.

In some embodiments of any of the methods or uses described herein, the cancer (e.g., CDK2-associated cancer) is a breast cancer selected from the group consisting of: estrogen receptor (ER)-positive/hormone receptor (HR)-positive breast cancer, HER2-negative breast cancer; ER-positive/HR-positive breast cancer, HER2-positive breast cancer; triple negative breast cancer (TNBC); and inflammatory breast cancer.

In some embodiments of any of the methods or uses described herein, the cancer (e.g., CDK2-associated cancer) is a breast cancer selected from the group consisting of: endocrine resistant breast cancer, trastuzumab-resistant breast cancer, and breast cancer demonstrating primary or acquired resistance to CDK4/CDK6 inhibition. In some embodiments, the breast cancer is advanced or metastatic breast cancer.

In some embodiments, the subject is a human.

Compounds of Formula (I) and pharmaceutically acceptable salts thereof are also useful for treating a CDK2-associated cancer. Accordingly, also provided herein is a method for treating a subject diagnosed with or identified as having a CDK2-associated cancer, e.g., any of the exemplary CDK2-associated cancers disclosed herein, comprising administering to the subject a therapeutically effective amount of a compound of Formula (I), or a pharmaceutically acceptable salt thereof, or a pharmaceutical composition thereof as defined herein.

In some aspects, provided herein is a method for treating cancer in a subject in need thereof, including administering to the subject a therapeutically effective amount of a compound of Formula (I), or a pharmaceutically acceptable salt thereof. Also provided is a method for treating a cancer in a subject in need thereof, including (I) identifying the cancer as being a CDK2-associated cancer; and (b) administering to the subject a therapeutically effective amount of a compound of Formula (I), or a pharmaceutically acceptable salt thereof.

Identifying the cancer identifying the cancer in the subject as a CDK2-associated cancer can be performed by any appropriate method. In some embodiments, the step of identifying the cancer in the subject as a CDK2-associated cancer includes performing an assay to detect dysregulation in a CDK2 gene, a CDK2 protein, or expression or activity or level of any of the same in a sample from the subject (e.g., CDK2, cyclin A2, cyclin E1, and/or cyclin E2. In some embodiments, the method further includes obtaining a sample from the subject (e.g., a biopsy sample). An assay can be any appropriate assay. In some embodiments, the assay is selected from the group consisting of sequencing (e.g., pyrosequencing or next generation sequencing), immunohistochemistry, enzyme-linked immunosorbent assay, and fluorescence in situ hybridization (FISH).

Also provided herein is a method for treating a cancer in a subject in need thereof, including administering to the subject a therapeutically effective amount of a compound of Formula (I), or a pharmaceutically acceptable salt thereof to a subject identified as having a CDK2-associated cancer.

Also provided herein is a method of treating a CDK2-associated cancer, comprising administering a therapeutically effective amount of a compound of Formula (I), or a pharmaceutically acceptable salt thereof, or a pharmaceutical composition comprising a compound of Formula (I), or a pharmaceutically acceptable salt thereof, and at least one pharmaceutically acceptable excipient, to a subject identified or diagnosed as having a CDK2-associated cancer.

Provided herein is also a method for treating cancer in a subject in need thereof, including: (I) determining that the cancer is associated with a dysregulation of a CDK2 gene, a CDK2 protein, or expression or activity or level of any of the same; and (b) administering to the subject a therapeutically effective amount of a compound of Formula (I), or a pharmaceutically acceptable salt thereof.

Provided herein is also a method for treating cancer in a subject in need thereof, including: (I) determining that the cancer is associated with a dysregulation of a cyclin A2 gene, a cyclin A2 protein, a cyclin E1 gene, a cyclin E1 protein, a cyclin E2 gene, a cyclin E2 protein, or expression or activity or level of any of the same (or a combination thereof); and (b) administering to the subject a therapeutically effective amount of a compound of Formula (I), or a pharmaceutically acceptable salt thereof.

Provided herein is also a method for treating cancer in a subject in need thereof, including: (I) determining that the cancer is associated with a dysregulation of a cyclin A2 gene, a cyclin A2 protein, or expression or activity or level of any of the same; and (b) administering to the subject a therapeutically effective amount of a compound of Formula (I), or a pharmaceutically acceptable salt thereof.

Provided herein is also a method for treating cancer in a subject in need thereof, including: (I) determining that the cancer is associated with a dysregulation of a cyclin E1 gene, a cyclin E1 protein, or expression or activity or level of any of the same; and (b) administering to the subject a therapeutically effective amount of a compound of Formula (I), or a pharmaceutically acceptable salt thereof.

Provided herein is also a method for treating cancer in a subject in need thereof, including: (I) determining that the cancer is associated with a dysregulation of a cyclin E2 gene, a cyclin E2 protein, or expression or activity or level of any of the same; and (b) administering to the subject a therapeutically effective amount of a compound of Formula (I), or a pharmaceutically acceptable salt thereof.

Determining that the cancer is associated with a dysregulation of a CDK2 gene, a CDK2 protein, a cyclin A2 gene, a cyclin A2 protein, a cyclin E1 gene, a cyclin E1 protein, a cyclin E2 gene, a cyclin E2 protein, or expression or activity or level of any of the same (or any combination thereof), can be performed using any appropriate method. In some embodiments, the step of determining that the cancer in the subject is a CDK2-associated cancer includes performing an assay to detect dysregulation in a CDK2 gene, a CDK2 protein, a cyclin A2 gene, a cyclin A2 protein, a cyclin E1 gene, a cyclin E1 protein, a cyclin E2 gene, a cyclin E2 protein, or expression or activity or level of any of the same (or any combination thereof), in a sample from the subject. In some embodiments, the method further includes obtaining a sample from the subject (e.g., a biopsy sample). An assay can be any appropriate assay. In some embodiments, the assay is selected from the group consisting of sequencing (e.g., pyrosequencing or next generation sequencing), immunohistochemistry, enzyme-linked immunosorbent assay, and fluorescence in situ hybridization (FISH).

Additionally provided herein is a method for treating a CDK2-associated cancer in a subject in need thereof, including administering to the subject a therapeutically effective amount of a compound of Formula (I), or a pharmaceutically acceptable salt thereof. Also provided is a method for treating cancer in a subject in need thereof, including: (I) identifying the cancer as being a CDK2-associated disease or disorder; and (b) administering to the subject a therapeutically effective amount of a compound of Formula (I), or a pharmaceutically acceptable salt thereof. In addition, provided herein is a method for treating cancer in a subject in need thereof, including: administering to the subject a therapeutically effective amount of a compound of Formula (I), or a pharmaceutically acceptable salt thereof to a subject identified as having a CDK2-associated cancer.

In some cases, compounds of Formula (I), or a pharmaceutically acceptable salt thereof can be useful for inhibiting the processes of cells, such as inhibiting the proliferation of cells. Accordingly, provided herein is a method for inhibiting mammalian cell proliferation, including contacting the mammalian cell with a compound of Formula (I), or a pharmaceutically acceptable salt thereof. Also provided herein is a method for inhibiting CDK2 activity in a mammalian cell, including contacting the mammalian cell with a compound of Formula (I), or a pharmaceutically acceptable salt thereof. In some embodiments, the contacting occurs in vivo. In some embodiments, the contacting occurs in vitro. A mammalian cell can be any appropriate cell. In some embodiments, the mammalian cell is a mammalian cancer cell. In some embodiments, the mammalian cancer cell is a mammalian CDK2-associated cancer cell. In some embodiments, the mammalian cell has dysregulation of a CDK2 gene, a CDK2 protein, a cyclin A2 gene, a cyclin A2 protein, a cyclin E1 gene, a cyclin E1 protein, a cyclin E2 gene, a cyclin E2 protein, or expression or activity or level of any of the same (or any combination thereof).

Compounds of Formula (I), or a pharmaceutically acceptable salt thereof can also be useful in the manufacture of medicaments, i.e., for use in the treatment of a CDK2-associated cancer.

In some embodiments of any of the methods or uses described herein, an assay used to determine whether the subject has a dysregulation of a gene (e.g., a CDK2, cyclin A2, cyclin E1, and/or cyclin E2 gene), or a protein (e.g., a CDK2, cyclin A2, cyclin E1, and/or cyclin E2 protein), or expression or activity or level of any of the same (or any combination thereof), using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of a gene (e.g., a CDK2, cyclin A2, cyclin E1, and/or cyclin E2 gene), or a protein (e.g., a CDK2, cyclin A2, cyclin E1, and/or cyclin E2 protein), or expression or activity or levels of any of the same (or any combination thereof). In some embodiments, the sample is a biological sample or a biopsy sample (e.g., a paraffin-embedded biopsy sample) from the subject. In some embodiments, the subject is a subject suspected of having a CDK2-associated cancer, a subject having one or more symptoms of a CDK2-associated cancer, and/or a subject that has an increased risk of developing a CDK2-associated cancer).

In some embodiments, dysregulation of a gene (e.g., a CDK2, cyclin A2, cyclin E1, and/or cyclin E2 gene), or a protein (e.g., a CDK2, cyclin A2, cyclin E1, and/or cyclin E2 protein), or the expression or activity or level of any of the same (or any combination thereof) can be identified using a liquid biopsy (variously referred to as a fluid biopsy or fluid phase biopsy). Liquid biopsy methods can be used to detect total tumor burden and/or the dysregulation of a gene (e.g., a CDK2, cyclin A2, cyclin E1, and/or cyclin E2 gene), or a protein (e.g., a CDK2, cyclin A2, cyclin E1, and/or cyclin E2 protein), or expression or activity or level of any of the same (or any combination thereof). Liquid biopsies can be performed on biological samples obtained relatively easily from a subject (e.g., via a simple blood draw) and are generally less invasive than traditional methods used to detect tumor burden and/or dysregulation of a gene (e.g., a CDK2, cyclin A2, cyclin E1, and/or cyclin E2 gene), or a protein (e.g., a CDK2, cyclin A2, cyclin E1, and/or cyclin E2 protein), or expression or activity or level of any of the same (or any combination thereof). In some embodiments, liquid biopsies can be used to detect the presence of dysregulation of a gene (e.g., a CDK2, cyclin A2, cyclin E1, and/or cyclin E2 gene), or a protein (e.g., a CDK2, cyclin A2, cyclin E1, and/or cyclin E2 protein), or expression or activity or level of any of the same (or any combination thereof), at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a gene (e.g., a CDK2, cyclin A2, cyclin E1, and/or cyclin E2 gene), or a protein (e.g., a CDK2, cyclin A2, cyclin E1, and/or cyclin E2 protein), or expression or activity or level of any of the same (or any combination thereof).

In the field of medical oncology, it is normal practice to use a combination of different forms of treatment to treat each subject with cancer. In medical oncology the other component(s) of such conjoint treatment or therapy in addition to compositions provided herein may be, for example, surgery, radiotherapy, and additional therapeutic agents such as those described herein.

For example, a surgery may be open surgery or minimally invasive surgery. Compounds of Formula (I), or a pharmaceutically acceptable salt thereof therefore may also be useful as adjuvants to cancer treatment, that is, they can be used in combination with one or more additional therapies or therapeutic agents, for example, a chemotherapeutic agent that works by the same or by a different mechanism of action.

In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof for a period of time and then undergo at least partial resection of the tumor. In some embodiments, the treatment with one or more doses of a compound of Formula (I), or a pharmaceutically acceptable salt thereof reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof for a period of time and undergo one or more rounds of radiation therapy. In some embodiments, the treatment with one or more doses of a compound of Formula (I), or a pharmaceutically acceptable salt thereof reduces the size of the tumor (e.g., the tumor burden) prior to the one or more rounds of radiation therapy.

In some embodiments, a compound of Formula (I), or a pharmaceutically acceptable salt thereof, can be used after administration of an additional therapeutic agent or additional therapy. For example, a subject in need thereof can be administered one or more doses of a compound of Formula (I), or a pharmaceutically acceptable salt thereof for a period of time after undergoing at least partial resection of the tumor. In some embodiments, the treatment with one or more doses of a compound of Formula (I), or a pharmaceutically acceptable salt thereof reduces the size (i.e. number of cells) of any remaining tumor after the at least partial resection of the tumor. In some embodiments, a subject in need thereof can be administered one or more doses of a compound of Formula (I), or a pharmaceutically acceptable salt thereof for a period of time after undergoing one or more rounds of radiation therapy. In some embodiments, the treatment with one or more doses of a compound of Formula (I), or a pharmaceutically acceptable salt thereof reduces the size (i.e. number of cells) of any remaining tumor after the one or more rounds of radiation therapy.

In some embodiments, a subject has a cancer (e.g., a locally advanced or metastatic tumor) that is refractory or intolerant to standard therapy (e.g., administration of a chemotherapeutic agent), such as a kinase inhibitor (e.g., a CDK4/CDK6 inhibitor such as palbociclib, ribociclib, or abemaciclib), immunotherapy, and/or radiation. In some embodiments, a subject has a cancer (e.g., a locally advanced or metastatic tumor) that has no standard therapy. In some embodiments, a subject is CDK2 inhibitor naïve. For example, the subject is naïve to treatment with a selective CDK2 inhibitor. In some embodiments, a subject is not CDK2 inhibitor naïve (i.e., the subject has been previously administered one or more CDK2 inhibitors). In some embodiments, a subject is CDK4/CDK6 inhibitor naïve. For example, the subject is naïve to treatment with a selective CDK4/CDK6 inhibitor. In some embodiments, a subject is not CDK4/CDK6 inhibitor naïve (i.e., the subject has been previously administered one or more CDK4/CDK6 inhibitors).

In some embodiments of any of the methods described herein, the compound of Formula (I), or a pharmaceutically acceptable salt thereof, may be administered in combination with a therapeutically effective amount of at least one additional therapeutic agent.

Non-limiting examples of additional therapeutic agents include: other kinase inhibitors (e.g., receptor tyrosine kinase-targeted therapeutic agents such as EGFR, HER2, MEK, RAF, or KRAS inhibitors), cytotoxic chemotherapeutics, angiogenesis inhibitors, and radiotherapy.

In some embodiments, the additional therapeutic agent is an epidermal growth factor receptor tyrosine kinase inhibitor (EGFR). For example, EGFR inhibitors can include osimertinib (merelectinib, Tagrisso), erlotinib (Tarceva), gefitinib (Iressa), cetuximab (Erbitux), necitumumab (Portrazza), neratinib (Nerlynx), lapatinib (Tykerb), panitumumab (Vectibix), and vandetanib (Caprelsa).

In some embodiments, the additional therapeutic agent is a HER2 inhibitor. Non-limiting examples of HER2 inhibitors include trastuzumab and pertuzumab.

In some embodiments, the additional therapeutic agent is a Ras-Raf-MEK-ERK pathway inhibitors (e.g., binimetinib, selumetinib, encorafenib, sorafenib, trametinib, and vemurafenib), PI3K-Akt-mTOR-S6K pathway inhibitors (e.g., everolimus, rapamycin, perifosine, temsirolimus), and other kinase inhibitors, such as baricitinib, brigatinib, capmatinib, danusertib, ibrutinib, milciclib, regorafenib, ruxolitinib, semaxanib, mobocertinib, avapritinib, fisogatinib, itacitinib, parsaclisib, pemigatinib, glesatinib, pexidartinib, rilzabrutinib, PF-477736 ((R)-amino-N-[5,6-dihydro-2-(1-methyl-1H-pyrazol-4-yl)-6-oxo-1H-pyrrolo[4,3,2-ef][2,3]benzodiazepin-8-yl]-cyclohexaneacetamide), PLX8394 ((3R)—N-[3-[5-(2-cyclopropylpyrimidin-5-yl)-1H-pyrrolo[2,3-b]pyridine-3-carbonyl]-2,4-difluorophenyl]-3-fluoropyrrolidine-1-sulfonamide), PRN1371 (8-(3-(4-acryloylpiperazin-1-yl) propyl)-6-(2,6-dichloro-3,5-dimethoxyphenyl)-2-(methylamino)pyrido[2,3-d]pyrimidin-7 (8H)-one), TG101209 (N-t-butyl-3-(5-methyl-2-(4-(4-methylpiperazin-1-yl)phenylamino)pyrimidin-4-ylamino)benzenesulfonamide), NMS-1286937, NMS-088, INCB52793, PLX7486, PLX9486, and INCB40093.

In some embodiments, the additional therapeutic agent is a cytotoxic chemotherapeutic. Non-limiting example of cytotoxic chemotherapeutics include bleomycin, bendamustine, fluorouracil, capecitabine, gemcitabine, vinorelbine, platinum agents such as carboplatin, oxaliplatin, or cisplatin, cyclophosphamide, cytarabine, dacarbazine, daunorubicin, doxorubicin, etoposide, irinotecan, lomustine, methotrexate, mitomycin C, pemetrexed, taxanes such as cabazitaxel, paclitaxel, or docetaxel, temozolomide, vinblastine, and vincristine.

In some embodiments, the additional therapeutic agent is an angiogenesis inhibitor, for example VEGF inhibitors, VEGFR inhibitors, TIE-2 inhibitors, PDGFR inhibitors, angiopoietin inhibitors, PKCβ inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors. Examples of specific angiogenesis inhibitors include, but are not limited to, sunitinib (Sutent), bevacizumab (Avastin), axitinib, SU-14813, AG-13958, vatalanib (CGP79787), sorafenib (Nexavar), pegaptanib octasodium (Macugen), vandetanib (Zactima), PF-0337210, SU-14843, AZD-2171, ranibizumab (Lucentis), Neovastat (AE941), tetrathiomolybdata (Coprexa), AMG706, VEGF Trap (AVE0005), CEP 7055, XL 880, telatinib, and CP-868,596. Other anti-angiogenesis agents include enzastaurin, midostaurin, perifosine, teprenone (Selbex) and UCN 01, lenalidomide (Revlimid), pomalidomide (Pomalyst), squalamine (Evizon), and thalidomide (Thalomid).

In some embodiments, the subject has a cancer that is known to be resistant to one of more of the additional therapies described herein. Accordingly, some embodiments provide a method of treating cancer in a subject in need thereof, comprising administering to the subject a therapeutically effective amount of a compound of Formula (I), or a pharmaceutically acceptable salt thereof, wherein the subject was previously administered one or more of a CDK4/CDK6 inhibitor (such as palbociclib, ribociclib, or abemaciclib), an endocrine therapy (such as fulvestrant, toremifene, anastrozole, exemestane, letrozole, and tamoxifen), a HER2 inhibitor (such as neratinib, trastuzumab, dacomitinib, lapatinib, tucatinib, pertuzumab, or margetuximab), cytotoxic chemotherapeutic, an EGFR, MEK, RAF or KRAS inhibitor, an inhibitor of the Ras-Raf-MEK-ERK pathway, or a combination of any of the foregoing.

In some embodiments, the subject was previously administered one or more of a CDK4/CDK6 inhibitor (such as palbociclib, ribociclib, or abemaciclib), an endocrine therapy (such as fulvestrant, toremifene, anastrozole, exemestane, letrozole, and tamoxifen), a cytotoxic chemotherapeutic (as described herein), an EGFR, MEK, RAF or KRAS inhibitor (as described herein), an inhibitor of the Ras-Raf-MEK-ERK pathway (as described herein), or a combination of any of the foregoing, and the previous therapy was unsuccessful in treating the cancer.

In some embodiments, the subject was previously administered a CDK4/CDK6 inhibitor (such as palbociclib, ribociclib, or abemaciclib), and an endocrine therapy (such as fulvestrant, toremifene, anastrozole, exemestane, letrozole, and tamoxifen), and the previous therapy was unsuccessful in treating the cancer. In some embodiments, the subject was previously administered a CDK4/CDK6 inhibitor (such as palbociclib, ribociclib, or abemaciclib) as a monotherapy, and the previous therapy was unsuccessful in treating the cancer.

### Methods of Inhibiting

Although the genetic basis of tumorigenesis may vary between different cancer types, the cellular and molecular mechanisms required for metastasis appear to be similar for all solid tumor types. During a metastatic cascade, the cancer cells lose growth inhibitory responses, undergo alterations in adhesiveness and produce enzymes that can degrade extracellular matrix components. This leads to detachment of tumor cells from the original tumor, infiltration into the circulation through newly formed vasculature, and/or migration and extravasation of the tumor cells at favorable distant sites where they may form colonies.

Accordingly, also provided herein are methods for inhibiting metastasis of a cancer in a subject having a cancer in need of such treatment (e.g., a subject at risk of developing metastasis), the method comprising administering to the subject a therapeutically effective amount of a compound of Formula (I), or a pharmaceutically acceptable salt thereof or a pharmaceutical composition thereof. In some embodiments, the cancer is a CDK2-associated cancer. In some embodiments, the compound of Formula (I), or a pharmaceutically acceptable salt thereof is used in combination with an additional therapy or another therapeutic agent, as described herein.

The term “metastasis” is an art known term and means the formation of an additional tumor (e.g., a solid tumor) at a site distant from a primary tumor in a subject, where the additional tumor includes the same or similar cancer cells as the primary tumor.

Also provided are methods of decreasing the risk of developing a metastasis or an additional metastasis in a subject having a CDK2-associated cancer that include: selecting, identifying, or diagnosing a subject as having a CDK2-associated cancer, and administering a therapeutically effective amount of a compound of Formula (I), or a pharmaceutically acceptable salt thereof to the subject selected, identified, or diagnosed as having a CDK2-associated cancer.

Also provided are methods of decreasing the risk of developing a metastasis or an additional metastasis in a subject having a CDK2-associated cancer that includes administering a therapeutically effective amount of a compound of Formula (I), or a pharmaceutically acceptable salt thereof to a subject having a CDK2-associated cancer. The decrease in the risk of developing a metastasis or an additional metastasis in a subject having a CDK2-associated cancer can be compared to the risk of developing a metastasis or an additional metastasis in the subject prior to treatment, or as compared to a subject or a population of subjects having a similar or the same CDK2-associated cancer that has received no treatment or a different treatment.

The phrase “risk of developing a metastasis” means the risk that a subject having a primary tumor will develop an additional tumor (e.g., a solid tumor) at a site distant from a primary tumor in a subject over a set period of time, where the additional tumor includes the same or similar cancer cells as the primary tumor. Methods for reducing the risk of developing a metastasis in a subject having a cancer are described herein.

The phrase “risk of developing additional metastases” means the risk that a subject having a primary tumor and one or more additional tumors at sites distant from the primary tumor (where the one or more additional tumors include the same or similar cancer cells as the primary tumor) will develop one or more further tumors distant from the primary tumor, where the further tumors include the same or similar cancer cells as the primary tumor. Methods for reducing the risk of developing additional metastasis are described herein.

Also provided is a method for inhibiting CDK2 activity in a mammalian cell, comprising contacting the mammalian cell with a compound of Formula (I). In some embodiments, the contacting is in vitro. In some embodiments, the contacting is in vivo. In some embodiments, the contacting is in vivo. In some embodiments, the mammalian cell is a mammalian cancer cell. In some embodiments, the mammalian cancer cell is any cancer as described herein. In some embodiments, the mammalian cancer cell is a CDK2-associated mammalian cancer cell. In some embodiments, the amount of the compound of Formula (I) is a therapeutically effective amount.

As used herein, the term “contacting” refers to the bringing together of indicated moieties in an in vitro system or an in vivo system. For example, “contacting” a cell with a compound provided herein includes the administration of a compound provided herein to a subject, such as a human, as well as, for example, introducing a compound provided herein into a sample containing a mammalian cellular or purified preparation containing the cell.

Also provided herein is a method of inhibiting mammalian cell proliferation, in vitro or in vivo, comprising contacting a mammalian cell with a compound of Formula (I). In some embodiments, the amount of the compound of Formula (I) is a therapeutically effective amount.

### Pharmaceutical Compositions and Kits

When employed as pharmaceuticals, compounds of Formula (I), including pharmaceutically acceptable salts thereof, can be administered in the form of pharmaceutical compositions comprising a compound of Formula (I), or a pharmaceutically acceptable salt thereof, and at least one pharmaceutically acceptable excipient. These compositions can be prepared n a manner known in the pharmaceutical art, and can be administered by a variety of routes, depending upon whether local or systemic treatment is desired and upon the area to be treated. Administration can be, for example, oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump.

Also provided herein are pharmaceutical compositions which contain, as the active ingredient, a compound of Formula (I) or pharmaceutically acceptable salt thereof, and one or more pharmaceutically acceptable excipients. For example, a pharmaceutical composition prepared using a compound of Formula (I) or a pharmaceutically acceptable salt thereof. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. In some embodiments, the composition is formulated for oral administration.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

The daily dosage of the compound of Formula (I) or a pharmaceutically acceptable salt thereof can be varied over a wide range from 1.0 to 10,000 mg per adult human per day, or any range therein.

Provided herein are pharmaceutical kits useful, for example, in the treatment of CDK2-associated diseases or disorders, such as cancer, which include one or more containers containing a pharmaceutical composition comprising a therapeutically effective amount of a compound provided herein. Such kits can further include, if desired, one or more of various pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit.

### EXAMPLES

### Preparation of Compounds

The starting materials used for the syntheses are either synthesized or obtained from commercial sources, such as, but not limited to, Sigma-Aldrich, Fluka, Acros Organics, Alfa Aesar, Enamine, Strem, VWR Scientific, and the like. Nuclear Magnetic Resonance (NMR) analysis was conducted using a Bruker AVANCE III HD (300 or 400) MHz spectrometer or Bruker AVANCE NEO 400 MHz spectrometer with an appropriate deuterated solvent. LCMS spectra were obtained on a Shimadzu LCMS-2020 with electrospray ionization in positive ion detection mode with 20ADXR pump, SIL-20ACXR autosampler, CTO-20AC column oven, M20A PDA Detector and LCMS 2020 MS detector.

The general methods for the preparation of the compounds of Formula (I) have been described in an illustrative manner and are intended to be descriptive, rather than limiting. Thus, it will be appreciated that conditions such as choice of solvent, temperature of reaction, volumes, reaction time may vary while still producing the desired compounds. In addition, it will be appreciated that many of the reagents provided in the following examples may be substituted with other suitable reagents. See, e.g., Smith & March, Advanced Organic Chemistry, 7th Ed. (2013). Such changes and modifications, including without limitation, those relating to the chemical structures, substituents, derivatives, intermediates, syntheses, formulations and/or methods of use provided herein, may be made without departing from the spirit and scope thereof.

### Example 1: Compound 89-(1¹S,1³R,Z)-4²-Fluoro-2¹H-9,14-dioxa-5-thia-3,6,12-triaza-2(5,3)-pyrazola-4(1,4)-benzena-1(1,3)-cyclopentanacyclotetradecaphan-13-one 5,5-dioxide

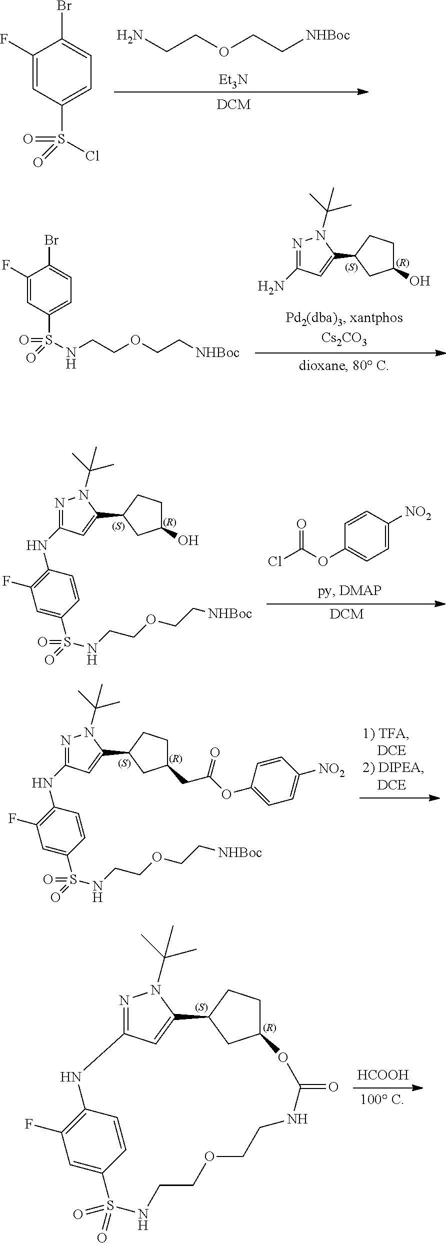

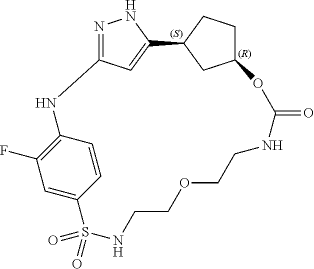

### Step 1. Synthesis of tert-butyl (2-(2-((4-bromo-3-fluorophenyl)sulfonamido)ethoxy)ethyl) carbamate

To a stirring solution of 4-bromo-3-fluorobenzene-1-sulfonyl chloride (200 mg, 731 μmol) in dichloromethane (2 mL) was added a solution of tert-butyl N-[2-(2-aminoethoxy)ethyl]carbamate (164 mg, 1.1 eq., 804 μmol) in dichloromethane (2 mL), followed by triethylamine (255 μL, 2.5 eq., 1.83 mmol) and the reaction was stirred overnight at room temperature. The reaction mixture was concentrated and then purified by column chromatography (20 to 60% EtOAc in hexanes) to afford tert-butyl(2-(2-((4-bromo-3-fluorophenyl)sulfonamido)ethoxy)ethyl)carbamate (340 mg, 100%) as a colorless oil. LC-MS: (ES+H, m/z) 463.1/465.1 [M+Na]⁺; ¹H NMR (400 MHz, CDCl₃) δ 7.78-7.70 (m, 1H), 7.66 (d, J=7.8 Hz, 1H), 7.58 (d, J=8.4 Hz, 1H), 5.54 (t, J=5.9 Hz, 1H), 4.87 (brs, 1H), 3.52 (t, J=5.0 Hz, 2H), 3.46 (t, J=5.1 Hz, 2H), 3.32-3.24 (m, 2H), 3.18 (q, J=5.1 Hz, 2H), 1.46 (s, 9H).

### Step 2. Synthesis of tert-butyl (2-(2-((4-((1-(tert-butyl)-5-((1S,3R)-3-hydroxycyclopentyl)-1H-pyrazol-3-yl)amino)-3-fluorophenyl)sulfonamido)ethoxy)ethyl)carbamate

A sealed vial with magnetic stirbar was charged with tert-butyl(2-(2-((4-bromo-3-fluorophenyl)sulfonamido)ethoxy)ethyl)carbamate (340 mg, 770 μmol), (1R,3S)-3-(3-amino-1-tert-butyl-1H-pyrazol-5-yl)cyclopentan-1-ol (189 mg, 1.1 eq., 847 μmol), Pd₂(dba)₃ (28.2 mg, 0.04 eq., 30.8 μmol), xantphos (35.7 mg, 0.08 eq., 61.6 μmol), cesium carbonate (502 mg, 2 eq., 1.54 mmol) and 1,4-dioxane (2.5 mL, 29.3 mmol). The resulting suspension was stirred under a bubbling stream of N₂ for 10 min and then stirred at 80° C. for 16 h. The mixture was cooled to room temperature, diluted with EtOAc, and washed with water. The aqueous layer waws extracted with EtOAc and the combined organics were washed with brine, dried, and concentrated under reduced pressure. The crude residue was purified by silica gel column chromatography (30 to 100% EtOAc in hexanes), and the product containing fractions were concentrated to afford tert-butyl (2-(2-((4-((1-(tert-butyl)-5-((1S,3R)-3-hydroxycyclopentyl)-1H-pyrazol-3-yl)amino)-3-fluorophenyl)sulfonamido)ethoxy)ethyl)carbamate (354 mg, 78.7%) as a colorless solid. LC-MS: (ES+H, m/z) 584.4 [M+H]⁺; ¹H NMR (400 MHz, CDCl₃) δ 7.57 (d, J=10.7 Hz, 1H), 7.49 (d, J=8.5 Hz, 1H), 6.75 (t, J=8.3 Hz, 1H), 5.98 (s, 1H), 5.70 (d, J=3.7 Hz, 1H), 5.15 (t, J=6.0 Hz, 1H), 4.90-4.82 (m, 1H), 4.72 (brs, 1H), 4.41-4.29 (m, 1H), 3.58 (brs, 1H), 3.49 (t, J=5.1 Hz, 2H), 3.43 (t, J=5.1 Hz, 2H), 3.29-3.20 (m, 2H), 3.13 (p, J=5.8 Hz, 2H), 2.18-2.08 (m, 1H), 2.07-1.72 (m, 5H), 1.58 (d, J=1.7 Hz, 9H), 1.43 (s, 9H).

### Step 3 Synthesis of tert-butyl (2-(2-((4-((1-(tert-butyl)-5-((1S,3R)-3-(((4-nitrophenoxy)carbonyl)oxy)cyclopentyl)-1H-pyrazol-3-yl)amino)-3-fluorophenyl)sulfonamido)ethoxy)ethyl)carbamate

To a stirring solution of tert-butyl(2-(2-((4-((1-(tert-butyl)-5-((1S,3R)-3-hydroxycyclopentyl)-1H-pyrazol-3-yl)amino)-3-fluorophenyl)sulfonamido)ethoxy)ethyl)carbamate (200 mg, 343 μmol) and 4-nitrophenyl carbonochloridate (76 mg, 1.1 eq., 377 μmol) in dichloromethane (4 mL) was added dropwise pyridine (55.2 μL, 2 eq., 685 μmol) followed by DMAP (2.1 mg, 0.05 eq., 17.1 μmol) and the reaction was stirred overnight at room temp. The mixture was diluted with DCM and washed with sat. NH₄Cl. The aqueous layer was extracted with DCM 2× and the combined organics were dried and evaporated. The crude residue was purified by silica gel chromatography (20 to 80% EtOAc in hexanes), product containing fractions were evaporated to afford tert-butyl(2-(2-((4-((1-(tert-butyl)-5-((1S,3R)-3-(((4-nitrophenoxy)carbonyl)oxy)cyclopentyl)-1H-pyrazol-3-yl)amino)-3-fluorophenyl)sulfonamido)ethoxy)ethyl)carbamate (1G, 119 mg, 46.4%) as a colorless foam. LC-MS: (ES+H, m/z) 749.3 [M+H]⁺.

### Step 4. Synthesis of (1¹S,1³R,Z)-2¹-(tert-butyl)-4²-fluoro-2¹H-9,14-dioxa-5-thia-3,6,12-triaza-2(5,3)-pyrazola-4(1,4)-benzena-1(1,3)-cyclopentanacyclotetradecaphan-13-one 5,5-dioxide

To a stirring solution of tert-butyl(2-(2-((4-((1-(tert-butyl)-5-((1S,3R)-3-(((4-nitrophenoxy)carbonyl)oxy)cyclopentyl)-1H-pyrazol-3-yl)amino)-3-fluorophenyl)sulfonamido)ethoxy)ethyl)carbamate (59 mg, 78.8 μmol) in DCE (3 mL) was added TFA (0.1 mL, 17 eq., 1.31 mmol) and the reaction was stirred overnight at room temperature. To the mixture was added DCE (12 mL) followed by DIPEA (274 μL, 20 eq., 1.58 mmol) and the reaction was stirred for an additional 18 h. The mixture was diluted with DCM, washed with sat. sodium bicarbonate, and extracted with DCM 3×. The combined organics were dried and concentrated. The crude residue was purified by silica gel chromatography (30 to 80% EtOAc in hexanes) to afford (1¹S,1³R,Z)-2¹-(tert-butyl)-4²-fluoro-2¹H-9,14-dioxa-5-thia-3,6,12-triaza-2(5,3)-pyrazola-4(1,4)-benzena-1(1,3)-cyclopentanacyclotetradecaphan-13-one 5,5-dioxide (25 mg, 62.3%) as a colorless oil. LC-MS: (ES+H, m/z) 510.3 [M+H]⁺.

### Step 5. (1¹S,1³R,Z)-4²-fluoro-2¹H-9,14-dioxa-5-thia-3,6,12-triaza-2(5,3)-pyrazola-4(1,4)-benzena-1(1,3)-cyclopentanacyclotetradecaphan-13-one 5,5-dioxide

Dissolved (1¹S,1³R,Z)-2¹-(tert-butyl)-4²-fluoro-2¹H-9,14-dioxa-5-thia-3,6,12-triaza-2(5,3)-pyrazola-4(1,4)-benzena-1(1,3)-cyclopentanacyclotetradecaphan-13-one 5,5-dioxide (25 mg, 49.1 μmol) in formic acid (0.5 mL, 270 eq., 13.3 mmol). The resulting solution was stirred for 16 h at 100° C. The reaction mixture was cooled and then concentrated under reduced pressure. The crude residue was purified by column (70 to 100% EtOAc in hexanes) and then by reverse phase chromatography (15 to 100% MeCN in water with 0.1% formic acid) to afford (1¹S,1³R,Z)-42-fluoro-2¹H-9,14-dioxa-5-thia-3,6,12-triaza-2(5,3)-pyrazola-4(1,4)-benzena-1(1,3)-cyclopentanacyclotetradecaphan-13-one 5,5-dioxide (8.5 mg, 38%) as an off white solid. LC-MS: (ES+H, m/z) 454.2 [M+H]⁺; ¹H NMR (400 MHz, MeOD-d₄) δ 7.65-7.51 (m, 2H), 7.25 (t, J=8.4 Hz, 1H), 6.69-6.35 (m, 1H), 6.07-5.94 (m, 1H), 5.14-5.06 (m, 1H), 3.41-3.03 (m, 9H), 2.45-2.32 (m, 1H), 2.30-2.16 (m, 1H), 2.11-1.86 (m, 4H).

Analytical data of compounds is shown in Table I below. The compounds in Table I were prepared using the procedure analogous to that disclosed above with appropriate modifications within the purview of one skilled in the art.

TABLE I
Compounds of Formula (I)

| Compound | | LCMS (ES + H, m/z); ¹H NMR |
|---|---|---|
| No. | Structure; IUPAC name | (ppm); ¹⁹F NMR (ppm) |
| 90 | 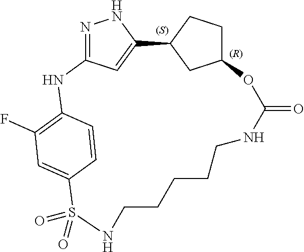 | 452.2 [M + H]⁺; ¹H NMR (400 MHz, MeOD-d₄) δ 7.52-7.46 (m, 2H), 7.18 (t, J = 8.3 Hz, 1H), 5.91 (s, 1H), 5.02 (brs, 1H), 3.29-3.25 (m, 2H), 3.06- 2.76 (m, 4H), 2.39-2.27 (m, 1H), 2.23-2.12 (m, 1H), 2.07- 1.75 (m, 4H), 1.29-1.00 (m, 6H). |
| | (1¹S,1³R,Z)-4²-fluoro-2¹H-14-oxa-5- | |
| | thia-3,6,12-triaza-2(5,3)-pyrazola- | |
| | 4(1,4)-benzena-1(1,3)- | |
| | cyclopentanacyclotetradecaphan-13- | |
| | one 5,5-dioxide | |
| 91 | 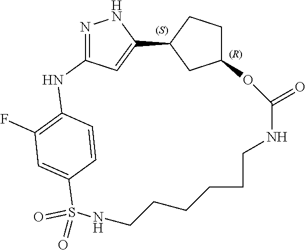 | 466.2 [M + H]⁺; ¹H NMR (400 MHz, MeOD-d₄) δ 7.56-7.46 (m, 2H), 7.36-7.18 (m, 1H), 5.98 (s, 1H), 5.22-5.01 (m, 1H), 4.10 (q, J = 7.1 Hz, 1H), 3.41-3.32 (m, 1H), 3.20- 2.73 (m, 4H), 2.47-2.30 (m, 1H), 2.29-2.15 (m, 1H), 2.08- 1.79 (m, 4H), 1.28-0.97 (m, 8H). |
| | (1¹S,1³R,Z)-4²-fluoro-2¹H-15-oxa-5- | |
| | thia-3,6,13-triaza-2(5,3)-pyrazola- | |
| | 4(1,4)-benzena-1(1,3)- | |
| | cyclopentanacyclopentadecaphan-14- | |
| | one 5,5-dioxide | |
| 92 | 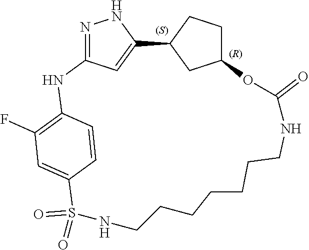 | 480.2 [M + H]⁺; ¹H NMR (400 MHz, DMSO-d₆) δ 12.14- 11.95 (m, 1H), 8.60-8.32 (m, 1H), 7.96-7.24 (m, 4H), 6.81- 6.40 (m, 1H), 5.81 (s, 1H), 5.08-4.81 (m, 1H), 3.32 (s, 5H), 2.30-2.11 (m, 1H), 2.05- 1.68 (m, 5H), 1.23-0.63 (m, 10H). |

### Example 2: Compound 93-(1¹S,1³R,Z)-2¹H-15-oxa-5-thia-3,6,13-triaza-2(5,3)-pyrazola-4(1,4)-cyclohexana-1(1,3)-cyclopentanacyclopentadecaphan-14-one 5,5-dioxide

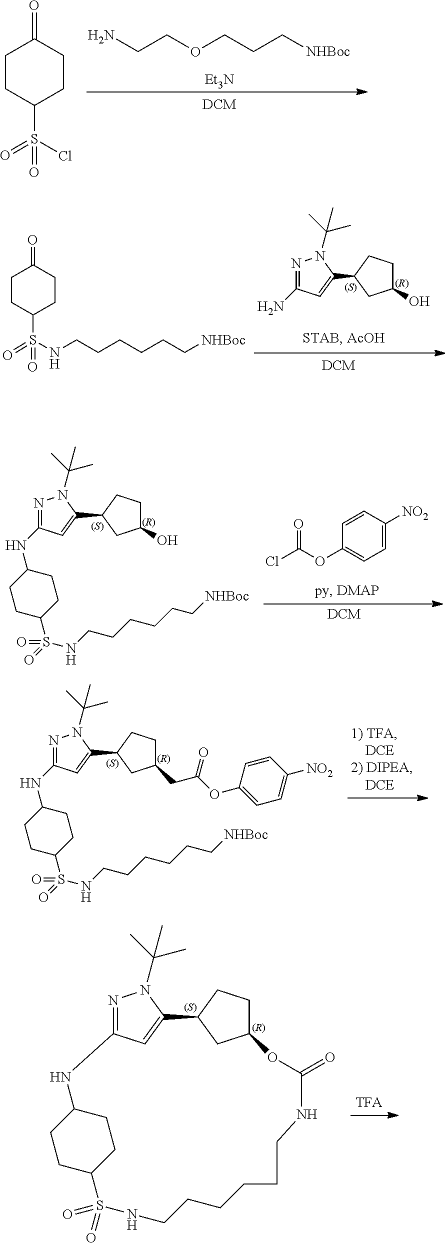

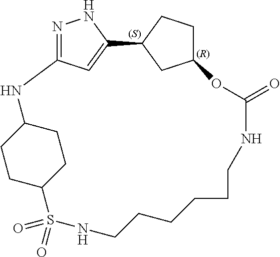

### Step 1: Synthesis of tert-butyl (6-((4-oxocyclohexane)-1-sulfonamido)hexyl)carbamate

To a solution of 4-oxocyclohexane-1-sulfonyl chloride (100 mg, 509 μmol, 1 equiv) and tert-butyl (6-aminohexyl)carbamate (132 mg, 610 μmol, 1.2 equiv) in DCM (5 mL) was added TEA (177 μL, 1.27 mmol, 2.5 equiv) and the resulting mixture was stirred overnight at room temperature. The reaction mixture was concentrated and purified by column chromatography (10 to 80% EtOAc in hexanes) to afford Synthesis of tert-butyl (6-((4-oxocyclohexane)-1-sulfonamido)hexyl)carbamate (111 mg, 58%) as a colorless foam. LC-MS: (ES+H, m/z) 277.2 [M+H-Boc]⁺; ¹H NMR (400 MHz, CDCl₃) δ 4.66 (d, J=55.5 Hz, 2H), 3.29 (td, J=11.0, 5.4 Hz, 1H), 3.19-3.01 (m, 4H), 2.63-2.51 (m, 2H), 2.50-2.31 (m, 4H), 2.15-1.99 (m, 2H), 1.56 (p, J=7.1 Hz, 2H), 1.51-1.28 (m, 15H).

### Step 2: Synthesis of tert-butyl (6-((4-((1-(tert-butyl)-5-((1S,3R)-3-hydroxycyclopentyl)-1H-pyrazol-3-yl)amino)cyclohexane)-1-sulfonamido)hexyl)carbamate

To a stirring solution of tert-butyl (6-((4-oxocyclohexane)-1-sulfonamido)hexyl)carbamate (111 mg, 295 μmol, 1 equiv) and (1R,3S)-3-(3-amino-1-(tert-butyl)-1H-pyrazol-5-yl)cyclopentan-1-ol (65.8 mg, 295 μmol, 1 equiv) in DCM (1.5 mL) was added acetic acid (42.2 μL, 737 μmol, 2.5 equiv), followed by sodium triacetoxyborohydride (93.7 mg, 295 μmol, 1.5 equiv) and the resulting mixture was stirred for 16 h at room temperature. An additional portion of sodium triacetoxyborohydride was added and the reaction was stirred for another 24 h. The reaction was diluted with DCM and washed with sat. NaHCO₃. The aqueous layer was extracted with DCM 3×. The combined organics were dried, and concentrated in vacuo. The crude residue was purified by column (20 to 100% EtOAc in hexanes) to afford tert-butyl (6-((4-((1-(tert-butyl)-5-((1S,3R)-3-hydroxycyclopentyl)-1H-pyrazol-3-yl)amino)cyclohexane)-1-sulfonamido)hexyl)carbamate (91 mg, 53%) as a colorless oil. LC-MS: (ES+H, m/z) 584.4 [M+H]⁺; ¹H NMR (400 MHz, CDCl₃) δ 5.35 (d, J=3.9 Hz, 1H), 4.63-4.39 (m, 4H), 4.31 (d, J=4.5 Hz, 1H), 3.65-3.55 (m, 1H), 3.48-3.30 (m, 1H), 3.28-2.76 (m, 10H), 2.33-2.15 (m, 3H), 2.15-1.72 (m, 10H), 1.70-1.18 (m, 22H).

### Step 3 Synthesis of tert-butyl (6-((4-((1-(tert-butyl)-5-((1S,3R)-3-(((4-nitrophenoxy)carbonyl)oxy)cyclopentyl)-1H-pyrazol-3-yl)amino)cyclohexane)-1-sulfonamido)hexyl)carbamate

To a stirring solution of tert-butyl (6-((4-((1-(tert-butyl)-5-((1S,3R)-3-hydroxycyclopentyl)-1H-pyrazol-3-yl)amino)cyclohexane)-1-sulfonamido)hexyl)carbamate (91 mg, 156 μmol, 1 equiv) and 4-nitrophenyl carbonochloridate (40.8 mg, 203 μmol, 1.3 equiv) in dichloromethane (1 mL) was added dropwise pyridine (31.4 μL, 390 μmol, 2.5 equiv) followed by DMAP (1.9 mg, 15.6 μmol, 0.1 equiv) and the reaction was stirred overnight at room temp. The mixture was diluted with DCM and washed with sat. NH₄Cl. The aqueous layer was extracted with DCM 2× and the combined organics were dried and evaporated. The crude residue was purified by silica gel chromatography (10 to 80% EtOAc in hexanes), product containing fractions were evaporated to afford tert-butyl (6-((4-((1-(tert-butyl)-5-((1S,3R)-3-(((4-nitrophenoxy)carbonyl)oxy)cyclopentyl)-1H-pyrazol-3-yl)amino)cyclohexane)-1-sulfonamido)hexyl)carbamate (67 mg, 57%) as a light yellow foam. LC-MS: (ES+H, m/z) 749.3 [M+H]⁺; ¹H NMR (400 MHz, CDCl₃) δ 8.27 (d, J=8.7 Hz, 2H), 7.38 (ddd, J=9.0, 4.3, 1.4 Hz, 2H), 5.43 (d, J=7.4 Hz, 1H), 5.26 (p, J=5.2 Hz, 1H), 4.54 (brs, 1H), 4.17 (brs, J=13.3, 6.8 Hz, 1H), 3.51-3.21 (m, 2H), 3.17-2.85 (m, 6H), 2.66-2.55 (m, 1H), 2.35-2.21 (m, 1H), 2.13-1.83 (m, 9H), 1.71-1.29 (m, 28H).

### Step 4: Synthesis of (1¹S,1³R,Z)-2¹H-15-oxa-5-thia-3,6,13-triaza-2(5,3)-pyrazola-4(1,4)-cyclohexana-1(1,3)-cyclopentanacyclopentadecaphan-14-one 5,5-dioxide

To a stirring solution of tert-butyl (6-((4-((1-(tert-butyl)-5-((1S,3R)-3-(((4-nitrophenoxy)carbonyl)oxy)cyclopentyl)-1H-pyrazol-3-yl)amino)cyclohexane)-1-sulfonamido)hexyl)carbamate (67 mg, 89.5 μmol, 1 equiv) in DCM (1 mL) was added TFA (137 μL, 1.79 mmol, 20 equiv) and the resulting solution was stirred for 16 h at room temperature. To the mixture was added DCE (20 mL) followed by DIPEA (374 μL, 2.68 mmol, 30 equiv) and the reaction was stirred for an additional 18 h. The mixture was diluted with DCM, washed with sat. sodium bicarbonate, and extracted with DCM 3×. The combined organics were dried and concentrated. The crude residue was purified by silica gel chromatography (30 to 100% EtOAc in hexanes) to afford (1¹S,1³R,Z)-2¹H-15-oxa-5-thia-3,6,13-triaza-2(5,3)-pyrazola-4(1,4)-cyclohexana-1(1,3)-cyclopentanacyclopentadecaphan-14-one 5,5-dioxide (23 mg, 50%) as a colorless oil. LC-MS: (ES+H, m/z) 510.4 [M+H]⁺.

### Step 5: Synthesis of (1¹S,1³R,Z)-4²-fluoro-2¹H-16-oxa-5-thia-3,6,14-triaza-2(5,3)-pyrazola-4(1,4)-benzena-1(1,3)-cyclopentanacyclohexadecaphan-15-one 5,5-dioxide

To a stirring solution of (1¹S,1³R,Z)-2¹H-15-oxa-5-thia-3,6,13-triaza-2(5,3)-pyrazola-4(1,4)-cyclohexana-1(1,3)-cyclopentanacyclopentadecaphan-14-one 5,5-dioxide (23 mg, 45.1 μmol, 1 equiv) in TFA (0.3 mL) was added water (15 μL) and the resulting solution was stirred for 18 h at 90° C. The mixture was cooled to room temperature and then concentrated in vacuo. The crude mixture was purified by column (60 to 100% EtOAc in hexanes) to afford (1¹S,1³R,Z)-42-fluoro-2¹H-16-oxa-5-thia-3,6,14-triaza-2(5,3)-pyrazola-4(1,4)-benzena-1(1,3)-cyclopentanacyclohexadecaphan-15-one 5,5-dioxide as an off-white solid. Yield: 10 mg, 49%; LC-MS: (ES+H, m/z) 454.3 [M+H]⁺; ¹H NMR (400 MHz, CDCl₃) δ 5.36 (d, J=6.5 Hz, 1H), 5.26-5.08 (m, 1H), 5.04-4.66 (m, 1H), 4.19-4.04 (m, 1H), 3.79-3.33 (m, 2H), 3.29-2.82 (m, 4H), 2.49-2.28 (m, 2H), 2.27-1.89 (m, 8H), 1.87-1.74 (m, 2H), 1.65-1.16 (m, 12H).

### Example 3: Compound 94-(11S,13R,24Z,64E)-21H,61H-13-oxa-5-thia-3,11-diaza-4(4,1)-piperidina-2(5,3),6(4,1)-dipyrazola-1(1,3)-cyclopentanacyclotridecaphan-12-one 5,5-dioxide

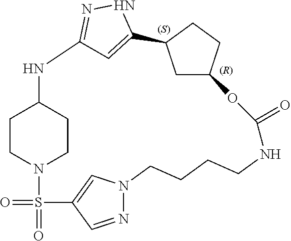

Step 1: tert-butyl 4-oxopiperidine-1-carboxylate (1.0 g, 5.0 mmol), (1R,3S)-3-(3-amino-1-(tert-butyl)-1H-pyrazol-5-yl)cyclopentan-1-ol (1.34 g, 1.2 eq., 6.02 mmol), 1,2-DCE (10 mL), and acetic acid (753 mg, 2.5 eq., 12.5 mmol) were added to a vial equipped with a stir bar and stirred at room temperature for 10 minutes. Sodium triacetoxyborohydride (2.13 g, 2 eq., 10 mmol) was added and the reaction was stirred for overnight at room temperature. At this point, the reaction was quenched with NaHCO₃, extracted with EtOAc, dried over sodium sulfate, concentrated under vacuum, and purified with column chromatography (silica, 120 g column, 0-100% EtOAc in hexanes) to afford tert-butyl 4-((1-(tert-butyl)-5-((1S,3R)-3-hydroxycyclopentyl)-1H-pyrazol-3-yl)amino)piperidine-1-carboxylate (1.04 g, 2.56 mmol, 51%) as a white solid. LC-MS (5-95% MeCN modified with 0.05% FA in 0.05% aq. FA, 5 min, ES-H, m/z): Calcd for [M+H] 407.3. Found: 407.4 (2.70 min). ¹H NMR (400 MHz, DMSO-d6) δ 5.42 (s, 1H), 4.63 (d, J=4.7 Hz, 1H), 4.29 (d, J=6.5 Hz, 1H), 4.16-4.07 (m, 1H), 3.89-3.78 (m, 2H), 3.24-3.06 (m, 1H), 2.85-2.76 (m, 1H), 2.17-2.08 (m, 1H), 1.90-1.62 (m, 5H), 1.60-1.46 (m, 12H), 1.45-1.32 (m, 12H).

Step 2: tert-butyl 4-((1-(tert-butyl)-5-((1S,3R)-3-hydroxycyclopentyl)-1H-pyrazol-3-yl)amino)piperidine-1-carboxylate (0.1 g, 250 μmol) was dissolved in dichloromethane (1 mL) and trifluoroacetic acid (200 μL, 11 eq., 2.6 mmol) was added. The reaction was stirred for one hour at room temperature. The crude reaction mixture was concentrated under vacuum and used without further purification. LC-MS (5-95% MeCN modified with 0.05% FA in 0.05% aq. FA, 5 min, ES-H, m/z): Calcd for [M+H] 307.2. Found: 307.3 (0.67 min). ¹H NMR (400 MHz, DMSO-d6) δ 8.52 (s, 1H), 8.27 (s, 1H), 5.84 (s, 1H), 4.28-4.11 (m, 1H), 3.42 (s, 1H), 3.35-3.24 (m, 2H), 3.11-2.81 (m, 3H), 2.29-2.12 (m, 1H), 2.07-1.96 (m, 3H), 1.94-1.82 (m, 1H), 1.81-1.67 (m, 4H), 1.64-1.46 (m, 11H).

Step 3: (1R,3S)-3-(1-(tert-butyl)-3-(piperidin-4-ylamino)-1H-pyrazol-5-yl)cyclopentan-1-ol (0.1 g, 238 μmol) and 1H-pyrazole-4-sulfonyl chloride (42 mg, 1.1 eq., 252 μmol) were suspended in dichloromethane (1 mL) and cooled to 0° C. Triethylamine (70 μL, 2 eq., 520 μmol) was added and the reaction was stirred at room temperature for one hour. At this point, LC-MS showed consumption of the starting material and the presence of product. The volatiles were removed under vacuum and the crude material was purified with column chromatography (silica, 12 g column, 0-10% MeOH in CH₂Cl₂) to afford (1R,3S)-3-(3-((1-((1H-pyrazol-4-yl)sulfonyl)piperidin-4-yl)amino)-1-(tert-butyl)-1H-pyrazol-5-yl)cyclopentan-1-ol (31 mg, 71 μmol, 37%) as a white solid. LC-MS (5-95% MeCN modified with 0.05% FA in 0.05% aq. FA, 5 min, ES-H, m/z): Calcd for [M+H] 437.2. Found: 437.3 (2.31 min). ¹H NMR (400 MHz, DMSO-d6) δ 13.74 (s, 1H), 8.08 (s, 2H), 5.42 (s, 1H), 4.59 (d, J=4.5 Hz, 1H), 4.22 (d, J=5.4 Hz, 1H), 4.10 (s, 1H), 3.30-3.20 (m, 2H), 3.21-3.12 (m, 1H), 3.11-2.98 (m, 1H), 2.86-2.71 (m, 1H), 2.57-2.54 (m, 1H), 2.17-2.03 (m, 1H), 1.96-1.84 (m, 2H), 1.83-1.57 (m, 5H), 1.56-1.45 (m, 2H), 1.43 (s, 9H).

Step 4: (1R,3S)-3-(3-((1-((1H-pyrazol-4-yl)sulfonyl)piperidin-4-yl)amino)-1-(tert-butyl)-1H-pyrazol-5-yl)cyclopentan-1-ol (29 mg, 66 μmol) and tert-butyl (4-bromobutyl)carbamate (17 mg, 66 μmol) were dissolved in acetonitrile (1.3 mL). cesium carbonate (65 mg, 3 eq., 200 μmol) was added and the reaction was stirred overnight at 60° C. At this point, LC-MS showed the consumption of starting material and the presence of product. The ACN was removed under vacuum and the reaction mixture was diluted with EtOAc, washed with brine, concentrated under vacuum and purified with column chromatography (silica, 12 g column 0-10% MeOH in CH₂Cl₂) to afford tert-butyl (4-(4-((4-((1-(tert-butyl)-5-((1S,3R)-3-hydroxycyclopentyl)-1H-pyrazol-3-yl)amino)piperidin-1-yl)sulfonyl)-1H-pyrazol-1-yl)butyl)carbamate (31 mg, 51 μmol, 77%) as a glassy white solid. LC-MS (5-95% MeCN modified with 0.05% FA in 0.05% aq. FA, 5 min, ES-H, m/z): Calcd for [M+H] 608.4. Found: 608.4 (2.78 min). ¹H NMR (400 MHz, DMSO-d6) δ 8.35 (s, 1H), 7.78 (s, 1H), 7.01-6.71 (m, 1H), 5.42 (s, 1H), 4.60 (d, J=4.7 Hz, 1H), 4.22 (d, J=5.3 Hz, 1H), 4.17 (t, J=7.0 Hz, 2H), 4.13-4.07 (m, 1H), 3.30-3.19 (m, 2H), 3.11-3.01 (m, 1H), 2.98-2.88 (m, 2H), 2.85-2.73 (m, 1H), 2.62-2.54 (m, 1H), 2.19-2.04 (m, 1H), 1.97-1.85 (m, 2H), 1.83-1.58 (m, 8H), 1.56-1.25 (m, 24H).

Step 5: tert-butyl (4-(4-((4-((1-(tert-butyl)-5-((1S,3R)-3-hydroxycyclopentyl)-1H-pyrazol-3-yl)amino)piperidin-1-yl)sulfonyl)-1H-pyrazol-1-yl)butyl)carbamate (108 mg, 178 μmol) was dissolved in dichloromethane (0.9 mL, 14.1 mmol). 4-nitrophenyl chloroformate (54 mg, 1.5 eq., 268 μmol) was added, followed by pyridine (30 μL, 2 eq., 380 μmol), then a catalytic amount of 4-dimethylaminopyridine. The reaction was stirred overnight at room temperature. At this point, LC-MS showed the presence of product. The crude reaction mixture was loaded directly onto a pre-packed silica cartridge and purified with column chromatography (silica, 12 g column, 0-100% EtOAc in CH₂Cl₂) to afford tert-butyl (4-(4-((4-((1-(tert-butyl)-5-((1S,3R)-3-(((4-nitrophenoxy)carbonyl)oxy)cyclopentyl)-1H-pyrazol-3-yl)amino)piperidin-1-yl)sulfonyl)-1H-pyrazol-1-yl)butyl)carbamate (89 mg, 116 μmol, 65%) as a cream-colored solid. LC-MS (5-95% MeCN modified with 0.05% FA in 0.05% aq. FA, 5 min, ES+H, m/z): Calcd for [M+H]+ 773.4. Found: 773.3 (3.46 min). ¹H NMR (400 MHz, DMSO-d6) δ 8.37-8.27 (m, 3H), 7.75 (s, 1H), 7.57-7.47 (m, 2H), 6.89-6.76 (m, 1H), 5.47 (s, 1H), 5.23-5.06 (m, 1H), 4.25 (d, J=5.4 Hz, 1H), 4.15 (t, J=7.0 Hz, 2H), 3.29-3.17 (m, 2H), 3.14-2.99 (m, 1H), 2.97-2.81 (m, 3H), 2.47-2.36 (m, 2H), 1.98-1.88 (m, 5H), 1.88-1.52 (m, 7H), 1.44 (s, 9H), 1.35 (s, 9H), 1.34-1.26 (m, 2H).

Step 6: Trifluoroacetic acid (120 μL, 14 eq., 1.57 mmol) was added to a stirred solution of tert-butyl (4-(4-((4-((1-(tert-butyl)-5-((1S,3R)-3-(((4-nitrophenoxy)carbonyl)oxy)cyclopentyl)-1H-pyrazol-3-yl)amino)piperidin-1-yl)sulfonyl)-1H-pyrazol-1-yl)butyl)carbamate (89 mg, 115 μmol) in 1,2-DCE (2 mL). The reaction was stirred at room temperature for 50 min. LC-MS showed removal of the Boc group. The reaction mixture was then diluted with 1,2-DCE (50 mL) and N,N-diisopropylethylamine (400 μL, 20 eq., 2.3 mmol) was added. The reaction was stirred at room temperature overnight. At this point, LC-MS showed the presence of product. The crude mixture was concentrated under vacuum and purified with column chromatography (silica, 12 g column, 0-100% EtOAc in CH₂Cl₂) to afford (1¹S,1³R,2⁴Z,6⁴E)-2¹-(tert-butyl)-2¹H,6¹H-13-oxa-5-thia-3,11-diaza-4(4,1)-piperidina-2(5,3),6(4,1)-dipyrazola-1(1,3)-cyclopentanacyclotridecaphan-12-one 5,5-dioxide (39 mg, 73 μmol, 63%) as a white solid. LC-MS (5-95% MeCN modified with 0.05% FA in 0.05% aq. FA, 5 min, ES+H, m/z): Calcd for [M+H]+ 534.3. Found: 534.3 (2.53 min). ¹H NMR (400 MHz, DMSO-d6) δ 8.39 (s, 1H), 7.80 (s, 1H), 7.15-6.79 (m, 1H), 5.30 (s, 1H), 4.98 (s, 1H), 4.43 (d, J=7.4 Hz, 1H), 4.24 (t, J=6.0 Hz, 2H), 3.56 (d, J=11.3 Hz, 2H), 3.03-2.95 (m, 1H), 2.94-2.72 (m, 3H), 2.25-2.12 (m, 1H), 2.05-1.89 (m, 5H), 1.84-1.73 (m, 3H), 1.67-1.50 (m, 5H), 1.47 (s, 9H), 1.15-1.06 (m, 2H).

Step 7: (1¹S,1³R,2⁴Z,6⁴E)-2¹-(tert-butyl)-2¹H,6¹H-13-oxa-5-thia-3,11-diaza-4(4,1)-piperidina-2(5,3),6(4,1)-dipyrazola-1(1,3)-cyclopentanacyclotridecaphan-12-one 5,5-dioxide (18 mg, 34 μmol) was dissolved in trifluoroacetic acid (0.2 mL). water (10 mg, 555 μmol) was added and the reaction was heated to 95° C. for 48 h. At this point, LC-MS showed starting material and product. The reaction mixture was then heated to 105° C. for 8 h. At this point, the reaction mixture was concentrated under vacuum and purified with column chromatography (silica, 4 g column, 0-10% MeOH in CH₂Cl₂) followed by HPLC (C18, 0-100% ACN in water with 5 mM HCl) to afford (1¹S,1³R,2⁴Z,6⁴E)-2¹H,6¹H-13-oxa-5-thia-3,11-diaza-4(4,1)-piperidina-2(5,3),6(4,1)-dipyrazola-1(1,3)-cyclopentanacyclotridecaphan-12-one 5,5-dioxide (6 mg, 12 μmol, 39%) as a white solid. LC-MS (5-95% MeCN modified with 0.05% FA in 0.05% aq. FA, 5 min, ES+H, m/z): Calcd for [M+H]+ 478.2. Found: 478.3 (2.28 min). ¹H NMR (400 MHz, DMSO-d6) δ 13.73 (s, 1H), 8.41 (s, 1H), 7.82 (s, 1H), 7.04 (s, 1H), 5.56 (s, 1H), 5.06 (s, 1H), 4.34-4.14 (m, 2H), 3.26-3.08 (m, 3H), 2.96-2.78 (m, 2H), 2.30-2.08 (m, 4H), 2.02-1.38 (m, 12H), 1.18-0.96 (m, 2H).

Analytical data of compounds is shown in Table II below. The compounds in Table II were prepared using the procedure analogous to that disclosed above with appropriate modifications within the purview of one skilled in the art.

TABLE II
Compounds of Formula (I)

| Compound | | LCMS (ES+H, m/z); ¹H NMR |
|---|---|---|
| No. | Structure; IUPAC name | (ppm); ¹⁹F NMR (ppm) |
| 95 | 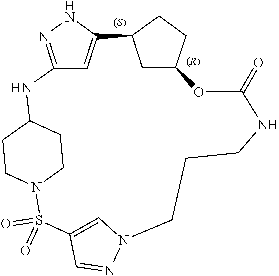 | [M + H] 464.2; Found: 464.3 (2.20 min). ¹H NMR (400 MHz, DMSO-d6) δ 11.14 (s, 1H), 8.49 (s, 1H), 7.81 (s, 1H), 7.13-6.77 (m, 1H), 5.09 (s, 1H), 4.90-4.61 (m, 2H), 4.36- 4.13 (m, 2H), 3.13-2.99 (m, 2H), 2.95-2.72 (m, 2H), 2.25- 2.01 (m, 4H), 1.99-1.38 (m, 12H). |
| | (1¹S, 1³R,2⁴Z,6⁴E)-2¹H,6¹H-12-oxa- | |
| | 5-thia-3,10-diaza-4(4,1)-piperidina- | |
| | 2(5,3),6(4,1)-dipyrazola-1(1,3)- | |
| | cyclopentanacyclododecaphan-11- | |
| | one 5,5-dioxide | |
| 96 | 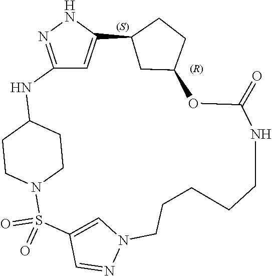 | [M + H] 492.2; Found: 492.3 (2.37 min). ¹H NMR (400 MHz, DMSO-d6) δ 11.50 (s, 1H), 8.37 (s, 1H), 7.79 (s, 1H), 7.25-6.58 (m, 1H), 5.33- 5.07 (m, 1H), 5.04-4.80 (m, 2H), 4.28-4.15 (m, 2H), 3.51- 3.41 (m, 1H), 3.09-2.99 (m, 2H), 2.98-2.76 (m, 2H), 2.29- 2.06 (m, 2H), 2.04-1.25 (m, 15H), 1.10-0.90 (m, 2H). |
| | (1¹S,1³R,2⁴Z,6⁴E)-2¹H,6¹H-14-oxa- | |
| | 5-thia-3,12-diaza-4(4,1)-piperidina- | |
| | 2(5,3),6(4,1)-dipyrazola-1(1,3)- | |
| | cyclopentanacyclotetradecaphan-13- | |
| | one 5,5-dioxide | |
| 97 | 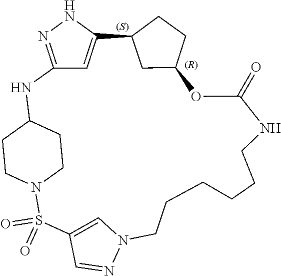 | [M + H] 506.3; Found: 506.3 (2.45 min). ¹H NMR (400 MHz, DMSO-d6) δ 11.22 (s, 1H), 8.48-8.27 (m, 1H), 7.80 (s, 1H), 7.13-6.75 (m, 1H), 5.24-5.12 (m, 1H), 4.92- 4.72 (m, 2H), 4.26-4.17 (m, 2H), 3.50-3.39 (m, 2H), 3.13- 2.97 (m, 1H), 2.96-2.76 (m, 3H), 2.31-2.14 (m, 3H), 2.11- 2.02 (m, 1H), 1.91-1.23 (m, 15H), 1.03-0.97 (m, 1H). |
| | (1¹S,1³R,2⁴Z,6⁴E)-2¹H,6¹H-15-oxa- | |
| | 5-thia-3,13-diaza-4(4,1)-piperidina- | |
| | 2(5,3),6(4,1)-dipyrazola-1(1,3)- | |
| | cyclopentanacyclopentadecaphan-14- | |
| | one 5,5-dioxide | |
| 98 | 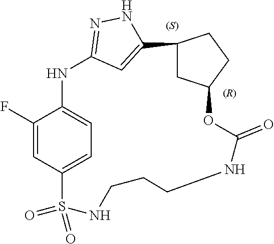 | [M + H]+ 424.1; Found: 424.2 (2.56 min). ¹H NMR (400 MHz, DMSO-d6) δ 11.92 (s, 1H), 8.19 (s, 1H), 7.65-7.29 (m, 3H), 7.16 (app t, J = 8.4 Hz, 1H), 6.84 (s, 1H), 5.53 (s, 1H), 4.86 (s, 1H), 3.26-3.13 (m, 1H), 3.06-2.82 (m, 2H), 2.81-2.70 (m, 1H), 2.65- 2.57 (m, 1H), 2.31-2.18 (m, 1H), 2.17-1.99 (m, 1H), 1.84 (d, J = 15.0 Hz, 1H), 1.79- 1.70 (m, 2H), 1.69-1.53 (m, 1H), 1.15-0.94 (m, 2H). |
| | (1¹S, 1³R,Z)-4²-fluoro-2¹H-12-oxa- | |
| | 5-thia-3,6, 10-triaza-2(5,3)-pyrazola- | |
| | 4(1,4)-benzena-1(1,3)- | |
| | cyclopentanacyclododecaphan-11- | |
| | one 5,5-dioxide | |
| 99 | 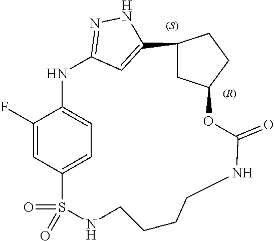 | [M + H]+ 438.2; Found: 438.3 (2.66 min). ¹H NMR (400 MHz, DMSO-d6, 70° C.) δ 11.82 (s, 1H), 8.01 (s, 1H), 7.61-7.31 (m, 2H), 7.24- 7.01 (m, 2H), 6.46 (s, 1H), 5.77 (s, 1H), 4.98 (s, 1H), 3.37-3.20 (m, 1H), 2.98-2.79 (m, 3H), 2.77-2.67 (m, 1H), 2.29-2.18 (m, 1H), 2.12-2.01 (m, 1H), 1.88-1.64 (m, 4H), 1.14-0.98 (m, 4H). |
| | (1¹S, 1³R,Z)-4²-fluoro-2¹H-13-oxa- | |
| | 5-thia-3,6,11-triaza-2(5,3)-pyrazola- | |
| | 4(1,4)-benzena-1(1,3)- | |
| | cyclopentanacyclotridecaphan-12-one | |
| | 5,5-dioxide | |
| 100 | 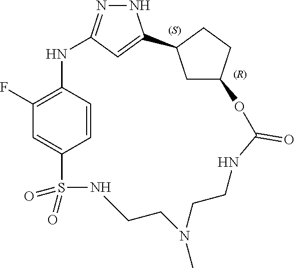 | [M + H] 467.2; Found: 467.2 (2.06 min). ¹H NMR (400 MHz, DMSO-d6, 60° C.) δ 11.98 (s, 1H), 8.18 (s, 1H), 7.61-7.48 (m, 1H), 7.48- 7.37 (m, 1H), 7.22-7.11 (m, 1H), 6.96 (s, 1H), 6.50 (s, 1H), 5.81 (s, 1H), 4.96 (s, 1H), 3.41-3.22 (m, 1H), 3.07-2.94 (m, 1H), 2.94-2.77 (m, 3H), 2.41-2.21 (m, 2H), 2.20- 1.98 (m, 7H), 1.95-1.71 (m, 4H). |
| | (1¹S, 1³R,Z)-4²-fluoro-9-methyl- | |
| | 2¹H-14-oxa-5-thia-3,6,9,12-tetraaza- | |
| | 2(5,3)-pyrazola-4(1,4)-benzena- | |
| | 1(1,3)- | |
| | cyclopentanacyclotetradecaphan-13- | |
| | one 5,5-dioxide | |
| 101 | 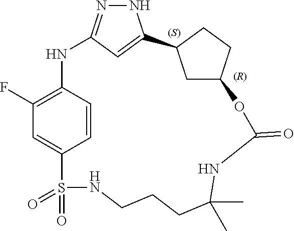 | [M + H] 466.2; Found: 466.2 (2.91 min). ¹H NMR (400 MHz, DMSO-d6) δ 11.99 (s, 1H), 8.35 (s, 1H), 7.69-7.47 (m, 1H), 7.47-7.29 (m, 2H), 7.28-7.11 (m, 1H), 6.40 (s, 1H), 5.83 (s, 1H), 5.15-4.67 (m, 1H), 3.27-3.13 (m, 1H), 3.03-2.75 (m, 2H), 2.31- 2.19 (m, 1H), 2.13-2.01 (m, 1H), 1.88-1.60 (m, 4H), 1.15- 1.07 (m, 7H), 1.05 (s, 3H). |
| | (1¹S,1³R,Z)-4²-fluoro-10,10- | |
| | dimethyl-2¹H-13-oxa-5-thia-3,6,11- | |
| | triaza-2(5,3)-pyrazola-4(1,4)- | |
| | benzena-1(1,3)- | |
| | cyclopentanacyclotridecaphan-12-one | |
| | 5,5-dioxide | |

### Example 5: Compound 102-(1¹S,1³R,2⁴Z)-4²-fluoro-2¹H-13-oxa-5-thia-3,6,11-triaza-2(5,3)-pyrazola-4(1,4)-benzena-1(1,3)-cyclopentanacyclotridecaphan-8-en-12-one 5,5-dioxide

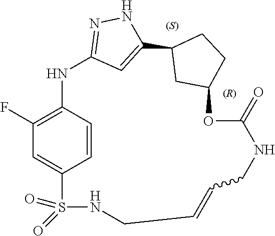

Step 1: To a stirred mixture of 4-bromo-3-fluorobenzenesulfonyl chloride (1 g, 3.66 mmol) and prop-2-en-1-amine hydrochloride (410 mg, 1.2 eq., 4.39 mmol) in THF, N,N-diisopropylethylamine (1.9 mL, 3 eq., 10 mmol) was added. The reaction was stirred at room temperature for 3.5 hours. The volatiles were removed under vacuum and the crude material was purified with column chromatography (silica, 120 g column, 0-60% EtOAc in hexanes) to afford N-allyl-4-bromo-3-fluorobenzenesulfonamide (306 mg, 1.04 mmol, 28%) as a white solid. LC-MS (5-95% MeCN modified with 0.05% FA in 0.05% aq. FA, 5 min, ES+H, m/z): Calcd for [M+H]+ 294.0. Found: 295.4 (3.33 min). ¹H NMR (400 MHz, DMSO) δ 8.18-7.82 (m, 2H), 7.74 (d, J=8.4 Hz, 1H), 7.57 (d, J=8.4 Hz, 1H), 5.85-5.51 (m, 1H), 5.15 (d, J=17.4 Hz, 1H), 5.04 (d, J=10.5 Hz, 1H), 3.48 (t, J=5.3 Hz, 2H).

Step 2 (1R,3S)-3-(3-amino-1-(tert-butyl)-1H-pyrazol-5-yl)cyclopentan-1-ol (0.3 g, 1.34 mmol), Xantphos (39 mg, 0.05 eq., 67 μmol), N-allyl-4-bromo-3-fluorobenzenesulfonamide (395 mg, 1.34 mmol), Pd₂(dba)₃ (62 mg, 0.05 eq., 67 μmol), and cesium carbonate (2.19 g, 5 eq., 6.72 mmol) were added to a vial equipped with a stir bar. The vial was evacuated and filled with nitrogen and 1,4-dioxane (7.5 mL) was added. The reaction was stirred for one hour at 80° C. At this point, LC-MS showed a large amount of starting material, (1R,3S)-3-(3-amino-1-(tert-butyl)-1H-pyrazol-5-yl)cyclopentan-1-ol. Additional N-allyl-4-bromo-3-fluorobenzenesulfonamide (100 mg, 0.34 mmol, 0.25 eq.) was added. The reaction was allowed to stir overnight at 80° C. At this point, LC-MS showed product and the consumption of starting material. The reaction was quenched with water and extracted with EtOAc (3×30 mL). The crude material was purified with column chromatography (silica, 80 g column, 0-100% EtOAc in hexanes) to afford N-allyl-4-((1-(tert-butyl)-5-((1S,3R)-3-hydroxycyclopentyl)-1H-pyrazol-3-yl)amino)-3-fluorobenzenesulfonamide (257 mg, 614 μmol, 45%) as a cream-colored solid. LC-MS (5-95% MeCN modified with 0.05% FA in 0.05% aq. FA, 5 min, ES+H, m/z): Calcd for [M+H]+ 437.2. Found: 437.3 (3.22 min). ¹H NMR (400 MHz, DMSO-d6) δ 8.10 (s, 1H), 7.59 (app t, J=6.0 Hz, 1H), 7.49 (d, J=11.3 Hz, 1H), 7.41 (d, J=8.3 Hz, 1H), 6.53 (t, J=8.5 Hz, 1H), 6.01 (s, 1H), 5.73-5.60 (m, 1H), 5.13 (d, J=17.4 Hz, 1H), 5.02 (d, J=10.2 Hz, 1H), 4.57 (d, J=4.4 Hz, 1H), 4.28-4.09 (m, 1H), 3.39 (t, J=5.9 Hz, 2H), 3.05-2.87 (m, 1H), 2.29-2.12 (m, 1H), 1.97-1.80 (m, 1H), 1.80-1.67 (m, 2H), 1.65-1.53 (m, 2H), 1.50 (s, 9H).

Step 3: N-allyl-4-((1-(tert-butyl)-5-((1S,3R)-3-hydroxycyclopentyl)-1H-pyrazol-3-yl)amino)-3-fluorobenzenesulfonamide (206 mg, 472 μmol), 4-nitrophenyl chloroformate (143 mg, 1.5 eq., 708 μmol), 1,2-dichloroethane (1 mL), pyridine (80 μL, 2.1 eq., 994 μmol), and DMAP (6 mg, 0.1 eq., 47 μmol) were added to a vial equipped with a stir bar. The reaction was stirred over night at room temperature. At this point, LC-MS showed the presence of product and the consumption of starting material. The reaction was concentrated under vacuum and the residue was purified with column chromatography (silica, 40 g column, 0-30% EtOAc in CH₂Cl₂) to afford (1R,3S)-3-(3-((4-(N-allylsulfamoyl)-2-fluorophenyl)amino)-1-(tert-butyl)-1H-pyrazol-5-yl)cyclopentyl (4-nitrophenyl) carbonate (198 mg, 329 μmol, 70%) as a colorless oil. LC-MS (5-95% MeCN modified with 0.05% FA in 0.05% aq. FA, 5 min, ES+H, m/z): Calcd for [M+H]+ 602.2. Found: 602.3 (3.82 min). ¹H NMR (400 MHz, DMSO-d6) δ 8.39-8.24 (m, 2H), 8.12 (s, 1H), 7.65-7.31 (m, 5H), 6.54 (t, J=8.5 Hz, 1H), 6.06 (s, 1H), 5.74-5.53 (m, 1H), 5.33-5.17 (m, 1H), 5.16-5.04 (m, 1H), 5.03-4.95 (m, 1H), 3.37 (t, J=6.0 Hz, 2H), 3.19-3.01 (m, 1H), 2.10-1.99 (m, 3H), 1.98-1.87 (m, 2H), 1.85-1.74 (m, 1H), 1.50 (s, 9H).

Step 4: (1R,3S)-3-(3-((4-(N-allylsulfamoyl)-2-fluorophenyl)amino)-1-(tert-butyl)-1H-pyrazol-5-yl)cyclopentyl (4-nitrophenyl) carbonate (241 mg, 401 μmol) and prop-2-en-1-amine hydrochloride (48 mg, 1.3 eq., 520 μmol) were dissolved in 1,2-DCE (2.5 mL). N,N-diisopropylethylamine (350 μL, 5 eq., 2 mmol) was added and the reaction was stirred overnight at room temperature. At this point, LC-MS showed consumption of starting material and the presence of product. The reaction was concentrated under vacuum and the residue was purified with column chromatography (silica, 40 g column, 20-70% EtOAc in hexanes) to afford (1R,3S)-3-(3-((4-(N-allylsulfamoyl)-2-fluorophenyl)amino)-1-(tert-butyl)-1H-pyrazol-5-yl)cyclopentyl allylcarbamate (164 mg, 316 μmol, 79%) as an oily white solid. ¹H NMR (400 MHz, DMSO-d6) δ 8.10 (s, 1H), 7.60 (app t, J=6.1 Hz, 1H), 7.49 (d, J=11.2 Hz, 1H), 7.40 (d, J=8.8 Hz, 1H), 7.31-7.10 (m, 1H), 6.52 (t, J=8.5 Hz, 1H), 6.04 (s, 1H), 5.87-5.60 (m, 2H), 5.26-4.82 (m, 5H), 3.71-3.51 (m, 2H), 3.41-3.35 (m, 2H), 3.13-2.95 (m, 1H), 2.45-2.34 (m, 1H), 2.04-1.92 (m, 1H), 1.92-1.78 (m, 1H), 1.78-1.58 (m, 3H), 1.49 (s, 9H). LC-MS (5-95% MeCN modified with 0.05% FA in 0.05% aq. FA, 5 min, ES+H, m/z): Calcd for [M+H]+ 520.2. Found: 520.3 (3.49 min).

Step 5: (1R,3S)-3-(3-((4-(N-allylsulfamoyl)-2-fluorophenyl)amino)-1-(tert-butyl)-1H-pyrazol-5-yl)cyclopentyl allylcarbamate (142 mg, 273 μmol) and Grubbs Cat. M204 (23 mg, 0.1 eq., 27 μmol) were added to a round bottom flask equipped with a stir bar. The flask was evacuated and filled with nitrogen 3×. 1,2 DCE (142 mL) was added and the reaction was stirred at room temperature overnight. At this point, LC-MS showed the presence of product and starting material. An additional 31 mg of Grubbs Cat. M204 (13 mol %) was added. The reaction was stirred and monitored over an additional 24 h during which two more additions (15 mg, followed by 17 mg) of catalyst were made. At this point, the solvent was removed under vacuum and the reaction was purified with reverse-phase chromatography (C18, 150 g column, 0-100% ACN in water, 0.1% HCOOH in both solvents), followed by HPLC (C18, 0-100% ACN in water with 5 mM HCl in both solvents) to afford (1¹S,1³R,2⁴Z)-2¹-(tert-butyl)-4²-fluoro-2¹H-13-oxa-5-thia-3,6,11-triaza-2(5,3)-pyrazola-4(1,4)-benzena-1(1,3)-cyclopentanacyclotridecaphan-8-en-12-one 5,5-dioxide (39 mg, 79 μmol, 29%, E/Z mixture) as a white solid. LC-MS (5-95% MeCN modified with 0.05% FA in 0.05% aq. FA, 5 min, ES-H, m/z): Calcd for [M+H] 492.2. Found: 492.2 (3.18 min). ¹H NMR (400 MHz, MeOD, 55° C.) δ 7.78-7.48 (m, 2H), 7.33-7.02 (m, 1H), 5.56-5.36 (m, 1H), 5.30-5.19 (m, 1H), 5.17 (s, 1H), 3.84-3.61 (m, 3H), 3.58-3.39 (m, 2H), 2.51-2.35 (m, 1H), 2.34-2.19 (m, 1H), 2.11-1.83 (m, 5H), 1.80 (s, 9H). ¹H NMR (400 MHz, MeOD, 23° C.) δ 7.65-7.32 (m, 2H), 7.26-6.99 (m, 1H), 6.16-5.83 (m, 1H), 5.47-5.30 (m, 1H), 5.29-5.09 (m, 1H), 5.07 (s, 1H), 3.76-3.53 (m, 3H), 3.47-3.33 (m, 2H), 2.51-2.03 (m, 2H), 1.99-1.74 (m, 4H), 1.70 (s, 9H). Note. exchangeable proton signals not seen.

Step 6: (1¹S,1³R,2⁴Z)-2¹-(tert-butyl)-4²-fluoro-2¹H-13-oxa-5-thia-3,6,11-triaza-2(5,3)-pyrazola-4(1,4)-benzena-1(1,3)-cyclopentanacyclotridecaphan-8-en-12-one 5,5-dioxide (51 mg, 104 μmol) was added to a vial equipped with a stir bar. Formic acid (0.5 mL) was added, and the reaction was stirred overnight at 95° C. At this point, the formic acid was removed under vacuum and the residue was re-dissolved in MeOH (0.5 mL) and 1N NaOH (1 mL) was added. The reaction was allowed to stir at room temperature for one hour. The reaction was diluted with brine and extracted with EtOAc (3×20 mL). The combined organic layers were dried over sodium sulfate, concentrated under vacuum, and purified with column chromatography (silica, 12 g column, 40-100% EtOAc in CH₂Cl₂) to afford (1¹S,1³R,2⁴Z)-4²-fluoro-2¹H-13-oxa-5-thia-3,6,11-triaza-2(5,3)-pyrazola-4(1,4)-benzena-1(1,3)-cyclopentanacyclotridecaphan-8-en-12-one 5,5-dioxide (29 mg, 67 μmol, 64%, E/Z mixture) as a white solid. LC-MS (5-95% MeCN modified with 0.05% FA in 0.05% aq. FA, 5 min, ES-H, m/z): Calcd for [M+H] 436.1. Found: 436.2 (2.67 min). ¹H NMR (400 MHz, DMSO-d6) δ 12.05 (s, 1H), 8.37 (s, 1H), 7.81-7.53 (m, 1H), 7.52-7.26 (m, 2H), 7.24-7.05 (m, 1H), 7.04-6.58 (m, 1H), 5.88 (s, 1H), 5.52-5.32 (m, 1H), 5.31-5.14 (m, 1H), 5.00 (s, 1H), 3.78-3.39 (m, 4H), 3.30-3.19 (m, 1H), 2.31-2.17 (m, 1H), 2.16-2.02 (m, 1H), 1.90-1.56 (m, 4H).

### Example 5: Compound 103—12-oxo-5,13-dioxa-3,11-diaza-2(5,2)-pyrimidina-4(1,3)-benzena-1(1,3)-cyclopentanacyclotridecaphane-44-sulfonamide

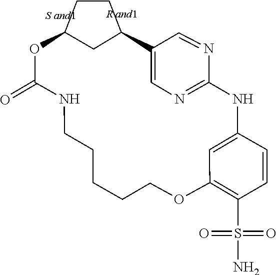 [SMILES: NS(=O)(=O)c1ccc2cc1OCCCCCNC(=O)O[C@H]1CC[C@H](C1)c1cnc(nc1)N2]

### Step 1: Preparation of 5-[3-[(tert-butyldimethylsilyl)oxy]cyclopentyl]pyrimidin-2-amine

To a stirred solution of 3-(2-aminopyrimidin-5-yl)cyclopentan-1-ol (2000 mg, 11.159 mmol, 1 equiv) and 1H-imidazole (1519.39 mg, 22.318 mmol, 2.00 equiv) in DCM (20 mL) at room temperature under nitrogen atmosphere. The resulting mixture was stirred for 30 min at room temperature under nitrogen atmosphere. To the above mixture was added TBSCl (2522.89 mg, 16.739 mmol, 1.50 equiv) in portions over 1 min at room temperature. The resulting mixture was stirred for additional 2 h at room temperature. Desired product could be detected by LCMS. The residue was purified by silica gel column chromatography, eluted with PE/EA (1:1-1:2) to afford rac-5-[(1R,3S)-3-[(tert-butyldimethylsilyl)oxy]cyclopentyl]pyrimidin-2-amine (1000 mg, 30.53%) as a white solid. LC-MS: (ES+H, m/z): [M+H]⁺=294.0.

### Step 2: Preparation of tert-butyl N-(5-{5-bromo-2-[(tert-butoxycarbonyl)aminosulfonyl]phenoxy}pentyl)carbamate

A solution of tert-butyl N-(5-hydroxypentyl)carbamate (172.18 mg, 0.847 mmol, 1.50 equiv) in THF (2 mL) was treated with NaH (45.17 mg, 1.130 mmol, 2.00 equiv, 60%) for 30 min at room temperature under nitrogen atmosphere followed by the addition of tert-butyl N-(4-bromo-2-fluorobenzenesulfonyl)carbamate (200 mg, 0.565 mmol, 1 equiv) in portions at room temperature. The resulting mixture was stirred for 1 h at room temperature under nitrogen atmosphere. Desired product could be detected by LCMS. The reaction was quenched by the addition of Water/Ice (2 mL) at room temperature. The resulting mixture was concentrated under reduced pressure. The residue was purified by reversed-phase flash chromatography with the following conditions: column, C18; mobile phase, MeCN in Water (10 mmol/L NH₄HCO₃), 15% to 50% gradient in 10 min; detector, UV 254 nm. The resulting mixture was concentrated under reduced pressure, to afford tert-butyl N-(5-{5-bromo-2-[(tert-butoxycarbonyl)aminosulfonyl]phenoxy}pentyl)carbamate (300 mg, 98.85%) as a colorless oil. LC-MS: (ES+H, m/z): [M−H]⁺=535.1/537.1.

### Step 3: Preparation of rac-tert-butyl N-(5-{2-[(tert-butoxycarbonyl)aminosulfonyl]-5-({5-[3-[(tert-butyldimethylsilyl)oxy]cyclopentyl]pyrimidin-2-yl}amino)phenoxy}pentyl)carbamate

To a stirred solution of 5-[(3-[(tert-butyldimethylsilyl)oxy]cyclopentyl]pyrimidin-2-amine (147.43 mg, 0.502 mmol, 1.00 equiv) and tert-butyl N-(5-{5-bromo-2-[(tert-butoxycarbonyl)aminosulfonyl]phenoxy}pentyl)carbamate (270 mg, 0.502 mmol, 1.00 equiv) in t-BuOH (4 mL) were added K₂CO₃ (208.28 mg, 1.506 mmol, 3 equiv) and EPhos Pd G4 (46.14 mg, 0.050 mmol, 0.1 equiv) in portions at room temperature under nitrogen atmosphere. The resulting mixture was stirred for 2 h at 100° C. under nitrogen atmosphere. Desired product could be detected by LCMS. The mixture was allowed to cool down to room temperature. The resulting mixture was filtered, the filter cake was washed with EtOAc (3×10 mL). The filtrate was concentrated under reduced pressure. The residue was purified by reversed-phase flash chromatography with the following conditions: column, C18; mobile phase, MeCN in Water (10 mmol/L NH₄HCO₃), 30% to 70% gradient in 10 min; detector, UV 254 nm. The resulting mixture was concentrated under reduced pressure, to afford tert-butyl N-(5-{2-[(tert-butoxycarbonyl)aminosulfonyl]-5-({5-[3-[(tert-butyldimethylsilyl)oxy]cyclopentyl]pyrimidin-2-yl}amino)phenoxy}pentyl)carbamate (180 mg, 47.77%) as a light yellow solid. LC-MS: (ES+H, m/z): [M+H]⁺=750.2. ¹H NMR (400 MHz, DMSO-d₆) δ 11.01 (s, 1H), 9.96 (s, 1H), 8.45 (s, 2H), 7.78 (s, 1H), 7.56 (d, J=8.8 Hz, 1H), 7.29 (d, J=9.0 Hz, 1H), 6.70 (s, 1H), 4.32 (s, 1H), 3.97 (t, J=6.6 Hz, 2H), 3.00-2.94 (m, 1H), 2.86 (d, J=6.3 Hz, 2H), 2.27-2.20 (m, 1H), 2.01-1.94 (m, 1H), 1.77-1.59 (m, 5H), 1.48 (dd, J=13.8, 7.8 Hz, 1H), 1.35 (s, 4H), 1.29 (s, 9H), 1.16 (s, 9H), 0.81 (s, 9H), 0.00 (s, 6H).

### Step 4: Preparation of tert-butyl N-(5-{2-[(tert-butoxycarbonyl)aminosulfonyl]-5-({5-[3-hydroxycyclopentyl]pyrimidin-2-yl}amino)phenoxy}pentyl)carbamate

To a stirred solution of tert-butyl N-(5-{2-[(tert-butoxycarbonyl)aminosulfonyl]-5-({5-[3-[(tert-butyldimethylsilyl)oxy]cyclopentyl]pyrimidin-2-yl}amino)phenoxy}pentyl)carbamate (170 mg, 0.227 mmol, 1 equiv) in Et₃N·₃HF (4 mL, 14.738 mmol) at room temperature under nitrogen atmosphere. The resulting mixture was stirred for 1 h at room temperature under nitrogen atmosphere. Desired product could be detected by LCMS. The mixture was basified to pH 9 with NH₃·H₂O. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by reversed-phase flash chromatography with the following conditions: column, C18; mobile phase, MeCN in Water (0.1% NH₃·H₂O), 10% to 40% gradient in 10 min; detector, UV 254 nm. The resulting mixture was concentrated under reduced pressure, to afford tert-butyl N-(5-{2-[(tert-butoxycarbonyl)aminosulfonyl]-5-({5-[3-hydroxycyclopentyl]pyrimidin-2-yl}amino)phenoxy}pentyl)carbamate (120 mg, 83.27%) as a grey solid. LC-MS: (ES+H, m/z): [M+H]⁺=636.3.

### Step 5: Preparation of 3-(2-{[3-({5-[(tert-butoxycarbonyl)amino]pentyl}oxy)-4-[(tert-butoxycarbonyl)aminosulfonyl]phenyl]amino}pyrimidin-5-yl)cyclopentyl 4-nitrophenyl Carbonate

To a stirred solution of tert-butyl N-(5-{2-[(tert-butoxycarbonyl)aminosulfonyl]-5-({5-[3-hydroxycyclopentyl]pyrimidin-2-yl}amino)phenoxy}pentyl)carbamate (120 mg, 0.189 mmol, 1 equiv) and DMAP (2.31 mg, 0.019 mmol, 0.10 equiv) in DCM (2 mL) were added DIEA (73.18 mg, 0.567 mmol, 3.00 equiv) and bis(4-nitrophenyl) carbonate (86.13 mg, 0.283 mmol, 1.5 equiv) in portions at room temperature under nitrogen atmosphere. The resulting mixture was stirred for overnight at room temperature under nitrogen atmosphere. Desired product could be detected by LCMS. The resulting mixture was concentrated under reduced pressure. The residue was purified by reversed-phase flash chromatography with the following conditions: column, C18; mobile phase, MeCN in Water, 30% to 70% gradient in 10 min; detector, UV 254 nm. The resulting mixture was concentrated under reduced pressure, to afford 3-(2-{[3-({5-[(tert-butoxycarbonyl)amino]pentyl}oxy)-4-[(tert-butoxycarbonyl)aminosulfonyl]phenyl]amino}pyrimidin-5-yl)cyclopentyl 4-nitrophenyl carbonate (130 mg, 86.00%) as a yellow solid. LC-MS: (ES+H, m/z): [M−H]⁺=799.3. ¹H NMR (400 MHz, DMSO-d₆) δ 11.09 (s, 1H), 10.04 (s, 1H), 8.54 (s, 2H), 8.38-8.26 (m, 2H), 7.78 (s, 1H), 7.66 (d, J=8.8 Hz, 1H), 7.62-7.54 (m, 2H), 7.47 (d, J=8.8 Hz, 1H), 6.77 (s, 1H), 5.26 (d, J=7.3 Hz, 1H), 3.09 (q, J=8.9 Hz, 1H), 2.95 (d, J=6.2 Hz, 2H), 2.64 (dt, J=14.4, 7.3 Hz, 1H), 2.17-2.02 (m, 3H), 1.94-1.74 (m, 4H), 1.43 (d, J=5.3 Hz, 4H), 1.37 (s, 9H), 1.24 (s, 9H), 1.19 (d, J=7.0 Hz, 1H), 0.91-0.70 (m, 1H).

### Step 6: Preparation of 3-[2-({3-[(5-aminopentyl)oxy]-4-sulfamoylphenyl}amino)pyrimidin-5-yl]cyclopentyl 4-nitrophenyl Carbonate

To a stirred solution of 3-(2-{[3-({5-[(tert-butoxycarbonyl)amino]pentyl}oxy)-4-[(tert-butoxycarbonyl)aminosulfonyl]phenyl]amino}pyrimidin-5-yl)cyclopentyl 4-nitrophenyl carbonate (70 mg, 0.087 mmol, 1 equiv) in HCOOH (3 mL) at room temperature under nitrogen atmosphere. The resulting mixture was stirred for 1 h at room temperature under nitrogen atmosphere. Desired product could be detected by LCMS. The resulting mixture was concentrated under reduced pressure, to afford 3-[2-({3-[(5-aminopentyl)oxy]-4-sulfamoylphenyl}amino)pyrimidin-5-yl]cyclopentyl 4-nitrophenyl carbonate (50 mg, crude) as a solid. LC-MS: (ES+H, m/z): [M+H]⁺=601.2.

### Step 7: Preparation of 7-oxo-6,14-dioxa-8,20,22,25-tetraazatetracyclo[19.2.2.1\hat{ }{2,5}.1\hat{ }{15,19}]heptacosa-1(23),15,17,19(26),21,24-hexaene-16-sulfonamide

To a stirred solution of 3-[2-({3-[(5-aminopentyl)oxy]-4-sulfamoylphenyl}amino)pyrimidin-5-yl]cyclopentyl 4-nitrophenyl carbonate (50 mg, 0.083 mmol, 1 equiv) in DMF (2 mL) was added DIEA (107.59 mg, 0.830 mmol, 10 equiv) dropwise at room temperature under nitrogen atmosphere. The resulting mixture was stirred for 2 h at 50° C. under nitrogen atmosphere. Desired product could be detected by LCMS. The mixture was allowed to cool down to room temperature. The residue was purified by reversed-phase flash chromatography with the following conditions: column, C18; mobile phase, MeCN in Water (10 mmol/L NH₄HCO₃), 15% to 55% gradient in 10 min; detector, UV 254 nm. The pure fraction was concentrated under reduced pressure and lyophilization, to afford 7-oxo-6,14-dioxa-8,20,22,25-tetraazatetracyclo[19.2.2.1\hat{ }{2,5}.1\hat{ }{15,19}]heptacosa-1(23),15,17,19(26),21,24-hexaene-16-sulfonamide (4.7 mg, 11.13%) as a white solid. LC-MS: (ES+H, m/z): [M+H]⁺=462.1. ¹H NMR (400 MHz, DMSO-d₆) δ 10.02 (s, 1H), 8.67 (s, 1H), 8.42 (s, 2H), 7.55 (d, J=8.6 Hz, 1H), 7.41 (d, J=8.1 Hz, 1H), 6.86-6.73 (m, 3H), 4.96-4.87 (m, 1H), 4.38 (d, J=6.2 Hz, 1H), 4.23-4.12 (m, 1H), 3.48-3.37 (m, 1H), 3.27-3.17 (m, 1H), 2.77-2.65 (m, 1H), 2.32 (d, J=15.8 Hz, 2H), 1.96-1.69 (m, 6H), 1.44 (d, J=42.6 Hz, 4H).

### Example 6: Compound 104 and Compound 105—rel-(2R,5S)-7-oxo-6,14-dioxa-8,20,22,25-tetraazatetracyclo[19.2.2.1\hat{ }{2,5}.1\hat{ }{15,19}]heptacosa-1(23),15,17,19(26),21,24-hexaene-16-sulfonamide and rel-(2S,5R)-7-oxo-6,14-dioxa-8,20,22,25-tetraazatetracyclo[19.2.2.1\hat{ }{2,5}.1\hat{ }{15,19}]heptacosa-1(23),15,17,19(26),21,24-hexaene-16-sulfonamide

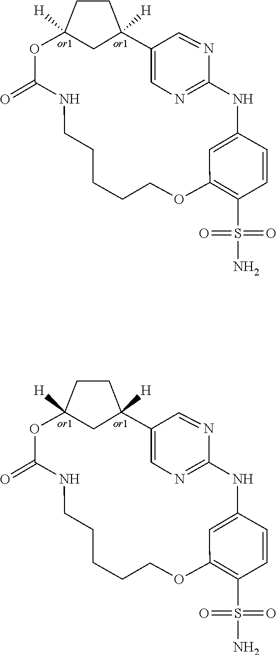 [SMILES: NS(=O)(=O)c1ccc2cc1OCCCCCNC(=O)O[C@H]1CC[C@H](C1)c1cnc(nc1)N2 ; NS(=O)(=O)c1ccc2cc1OCCCCCNC(=O)O[C@@H]1CC[C@@H](C1)c1cnc(nc1)N2]

### Step 1: Preparation of (isomer 1) rel-(2R,5S)-7-oxo-6,14-dioxa-8,20,22,25-tetraazatetracyclo[19.2.2.1\hat{ }{2,5}1\hat{ }{15,19}]heptacosa-1(23),15,17,19(26),21,24-hexaene-16-sulfonamide & (Isomer 2) rel-(2S,5R)-7-oxo-6,14-dioxa-8,20,22,25-tetraazatetracyclo[19.2.2.1\hat{ }{2,5}.1\hat{ }{15,19}]heptacosa-1(23),1e5,17,19(26),21,24-hexaene-16-sulfonamide

rac-(cis)-12-oxo-5,13-dioxa-3,11-diaza-2(5,2)-pyrimidina-4(1,3)-benzena-1(1,3)-cyclopentanacyclotridecaphane-44-sulfonamide (50 mg) was isolated by Prep-Chiral-HPLC with the following conditions (Column: Enantiocel C9-5, 3.0*25 MM; Mobile Phase A: Hex (10 mM NH₃-MeOH), Mobile Phase B: IPA; Flow rate: 50 mL/min; Gradient: isocratic 50; Wave Length: 300/282 nm; RT1 (min): 28.785; RT2 (min): 38.368). The pure fraction was concentrated under reduced pressure and by lyophilization to afford (isomer 1) rel-(2R,5S)-7-oxo-6,14-dioxa-8,20,22,25-tetraazatetracyclo[19.2.2.1\hat{ }{2,5}.1\hat{ }{15,19}]heptacosa-1(23),15,17,19(26),21,24-hexaene-16-sulfonamide (pre-peak, 13.8 mg, 25.70%) as a white solid and (isomer 2) rel-(2S,5R)-7-oxo-6,14-dioxa-8,20,22,25-tetraazatetracyclo[19.2.2.1\hat{ }{2,5}.1\hat{ }{15,19}]heptacosa-1 (23),15,17,19(26),21,24-hexaene-16-sulfonamide (post-peak, 12.1 mg, 23.11%) as a white solid.

Compound 104: LC-MS: (ES+H, m/z): [M+H]⁺=462.2. ¹H NMR (400 MHz, DMSO-d₆) δ 10.02 (s, 1H), 8.67 (s, 1H), 8.42 (s, 2H), 7.54 (d, J=8.6 Hz, 1H), 7.41 (d, J=8.8 Hz, 1H), 6.91-6.71 (m, 3H), 4.98-4.85 (m, 1H), 4.36 (d, J=12.3 Hz, 1H), 4.24-4.14 (m, 1H), 3.48-3.38 (m, 1H), 3.26-3.17 (m, 1H), 2.77-2.65 (m, 1H), 2.31 (d, J=15.8 Hz, 2H), 1.96-1.70 (m, 6H), 1.43 (d, J=43.6 Hz, 4H).

Compound 105: LC-MS: (ES+H, m/z): [M+H]⁺=462.2. ¹H NMR (400 MHz, DMSO-d₆) δ 10.02 (s, 1H), 8.67 (s, 1H), 8.42 (s, 2H), 7.54 (d, J=8.6 Hz, 1H), 7.41 (d, J=8.9 Hz, 1H), 6.77 (d, J=23.1 Hz, 3H), 4.95-4.86 (m, 1H), 4.43-4.13 (m, 2H), 3.48-3.38 (m, 1H), 3.21 (d, J=9.9 Hz, 1H), 2.75-2.64 (m, 1H), 2.31 (d, J=16.9 Hz, 2H), 1.97-1.69 (m, 6H), 1.44 (d, J=44.3 Hz, 4H).

### Example 8: Compound 106—7-oxo-6-oxa-8,14,20,22,25-pentaazatetracyclo[19.2.2.1\hat{ }{2,5}.1\hat{ }{15,19}]heptacosa-1(23),15,17,19(26),21,24-hexaene-16-sulfonamide

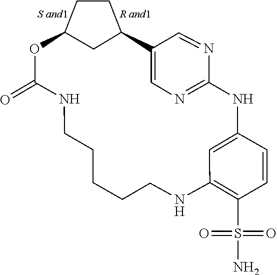 [SMILES: NS(=O)(=O)c1ccc2cc1NCCCCCNC(=O)O[C@H]1CC[C@H](C1)c1cnc(nc1)N2]

### Step 1: Preparation of 5-[3-[(tert-butyldimethylsilyl)oxy]cyclopentyl]pyrimidin-2-amine

A solution of 3-(2-aminopyrimidin-5-yl)cyclopentan-1-ol (2 g, 11.16 mmol, 1.00 equiv.) and Imidazole (1.90 g, 27.91 mmol, 2.50 equiv.) in DCM (10 mL) was stirred for 30 min at room temperature under nitrogen atmosphere. To the above mixture was added TBSCl (2.19 g, 14.51 mmol, 1.30 equiv.) in DCM (5 mL) dropwise over 5 min at room temperature. The resulting mixture was stirred for additional 2 h at room temperature. The reaction was monitored by LCMS. Desired product could be detected by LCMS. The resulting mixture was extracted with DCM (3×30 mL). The combined organic layers were washed with water (3×10 mL), brine (3×10 mL), dried over anhydrous Na₂SO₄. After filtration, the filtrate was concentrated under reduced pressure to afford 5-[3-[(tert-butyldimethylsilyl)oxy]cyclopentyl]pyrimidin-2-amine (2.3 g, crude) as a white solid. The crude product was used in the next step directly without further purification. LC-MS: (ES+H, m/z): [M+H]⁺=294.05.

### Step 2: Preparation of N-[5-({2-[(tert-butoxycarbonyl)aminosulfonyl]-5-({5-[3-[(tert-butyldimethylsilyl)oxy]cyclopentyl]pyrimidin-2-yl}amino)phenyl}amino)pentyl]carbamate

To a mixture of 5-[3-[(tert-butyldimethylsilyl)oxy]cyclopentyl]pyrimidin-2-amine (300 mg, 1.02 mmol, 1.00 equiv.) and tert-butyl N-[5-({5-bromo-2-[(tert-butoxycarbonyl)aminosulfonyl]phenyl}amino)pentyl]carbamate (822.58 mg, 1.53 mmol, 1.50 equiv.) in t-BuOH (6 mL) were added EPhos Pd G4 (93.90 mg, 0.10 mmol, 0.10 equiv.) and K₂CO₃ (423.82 mg, 3.07 mmol, 3.00 equiv.) at room temperature under nitrogen atmosphere. The resulting mixture was stirred for 3 h at 100° C. under nitrogen atmosphere. The reaction was monitored by LCMS. Desired product could be detected by LCMS. The mixture was allowed to cool down to room temperature. The resulting mixture was extracted with EtOAc (3×20 mL). The combined organic layers were washed with water (3×10 mL), dried over anhydrous Na₂SO₄. After filtration, the filtrate was concentrated under reduced pressure. The residue was purified by reversed-phase combi-flash chromatography with the following conditions: column, C18; mobile phase, MeCN in Water (10 mmol/L NH₄HCO₃), 35% to 50% gradient in 10 min; detector, UV 254 nm. The pure fraction was concentrated under reduced pressure to afford tert-butyl N-[5-({2-[(tert-butoxycarbonyl)aminosulfonyl]-5-({5-[3-[(tert-butyldimethylsilyl)oxy]cyclopentyl]pyrimidin-2-yl}amino)phenyl}amino)pentyl]carbamate (350 mg, 45.71%) as a light brown solid. LC-MS: (ES+H, m/z): [M+H]⁺=749.35.

### Step 3: Preparation of tert-butyl N-[5-({2-[(tert-butoxycarbonyl)aminosulfonyl]-5-({5-[(3-hydroxycyclopentyl]pyrimidin-2-yl}amino)phenyl}amino)pentyl]carbamate

A solution of tert-butyl N-[5-({2-[(tert-butoxycarbonyl)aminosulfonyl]-5-({5-[3-[(tert-butyldimethylsilyl)oxy]cyclopentyl]pyrimidin-2-yl}amino)phenyl}amino)pentyl]carbamate (350 mg, 0.47 mmol, 1.00 equiv.) in Et₃N·3HF (4 mL) were stirred for 3 h at room temperature under nitrogen atmosphere. The reaction was monitored by LCMS. Desired product could be detected by LCMS. The resulting mixture was concentrated under reduced pressure. The mixture neutralized to pH 7 with NH₃·H₂O. The residue was purified by reversed-phase combi-flash chromatography with the following conditions: column, C18; mobile phase, MeCN in Water (10 mmol/L NH₄HCO₃), 45% to 65% gradient in 10 min; detector, UV 254 nm. The pure fraction was concentrated under reduced pressure to afford tert-butyl N-[5-({2-[(tert-butoxycarbonyl)aminosulfonyl]-5-({5-[3-hydroxycyclopentyl]pyrimidin-2-yl}amino)phenyl}amino)pentyl]carbamate (180 mg, 60.69%) as a light yellow solid. LC-MS: (ES+H, m/z): [M+H]⁺=635.25. ¹H NMR (400 MHz, DMSO-d₆) δ 11.23 (s, 1H), 9.78 (s, 1H), 8.48 (s, 2H), 7.51-7.39 (m, 2H), 7.12-7.05 (m, 1H), 6.78 (s, 1H), 5.78 (s, 1H), 4.69 (d, J=3.8 Hz, 1H), 4.23 (d, J=5.2 Hz, 1H), 3.18-3.14 (m, 2H), 2.95-2.91 (m, 3H), 2.30-2.25 (m, 1H), 1.99-1.94 (m, 1H), 1.79-1.39 (m, 10H), 1.37 (s, 9H), 1.30 (s, 9H).

### Step 4: Preparation of 3-(2-{[3-({5-[(tert-butoxycarbonyl)amino]pentyl}amino)-4-[(tert-butoxycarbonyl)aminosulfonyl]phenyl]amino}pyrimidin-5-yl)cyclopentyl 4-nitrophenyl carbonate

To a mixture of tert-butyl N-[5-({2-[(tert-butoxycarbonyl)aminosulfonyl]-5-({5-[3-hydroxycyclopentyl]pyrimidin-2-yl}amino)phenyl}amino)pentyl]carbamate (180 mg, 0.28 mmol, 1.00 equiv.) and DMAP (3.46 mg, 0.03 mmol, 0.10 equiv.) in DCM (6 mL) were added DIEA (109.95 mg, 0.85 mmol, 3.00 equiv.) and bis(4-nitrophenyl) carbonate (172.52 mg, 0.57 mmol, 2.00 equiv.) at room temperature under nitrogen atmosphere. The resulting mixture was stirred for overnight at room temperature under nitrogen atmosphere. The reaction was monitored by LCMS. Desired product could be detected by LCMS. The resulting mixture was concentrated under reduced pressure. The residue was purified by reversed-phase flash chromatography with the following conditions: column, C18; mobile phase, MeCN in Water, 55% to 70% gradient in 10 min; detector, UV 254 nm. The pure fraction was concentrated under reduced pressure to afford 3-(2-{[3-({5-[(tert-butoxycarbonyl)amino]pentyl}amino)-4-[(tert-butoxycarbonyl)aminosulfonyl]phenyl]amino}pyrimidin-5-yl)cyclopentyl 4-nitrophenyl carbonate (190 mg, 83.77%) as a light yellow solid. LC-MS: (ES+H, m/z): [M+H]⁺=800.30.

### Step 5/6: Preparation of 7-oxo-6-oxa-8,14,20,22,25-pentaazatetracyclo[19.2.2.1\hat{ }{2,5}.1\hat{ }{15,19}]heptacosa-1(23),15,17,19(26),21,24-hexaene-16-sulfonamide

A solution of 3-(2-{[3-({5-[(tert-butoxycarbonyl)amino]pentyl}amino)-4-[(tert-butoxycarbonyl)aminosulfonyl]phenyl]amino}pyrimidin-5-yl)cyclopentyl 4-nitrophenyl carbonate (180 mg, 0.23 mmol, 1.00 equiv.) in HCOOH (4 mL) were stirred for overnight at room temperature under nitrogen atmosphere. Desired product could be detected by LCMS. The resulting mixture was concentrated under reduced pressure. The mixture was dissolved in DMF (3 mL) and neutralized to pH 8 with DIEA. The resulting mixture was stirred for 3 h at 50° C. under nitrogen atmosphere. Desired product could be detected by LCMS. The mixture was allowed to cool down to room temperature. The resulting mixture was extracted with EtOAc (3×10 mL). The combined organic layers were washed with water (3×5 mL), dried over anhydrous Na₂SO₄. After filtration, the filtrate was concentrated under reduced pressure. The residue was purified by reversed-phase combi-flash chromatography with the following conditions: column, C18; mobile phase, MeCN in Water (0.1% NH₃·H₂O+10 mmol/L NH₄HCO₃), 30% to 50% gradient in 10 min; detector, UV 254 nm. The pure fraction was concentrated under reduced pressure and lyophilization to afford 7-oxo-6-oxa-8,14,20,22,25-pentaazatetracyclo[19.2.2.1\hat{ }{2,5}.1\hat{ }{15,19}]heptacosa-1(23),15,17,19(26),21,24-hexaene-16-sulfonamide (41.0 mg, 39.54%) as a white solid. LC-MS: (ES+H, m/z) 461.1 [M+H]⁺. ¹H NMR (400 MHz, DMSO-d₆) δ 9.73 (s, 1H), 8.38-8.26 (m, 3H), 7.42 (d, J=8.7 Hz, 2H), 6.85 (brs, 1H), 6.42 (dd, J=8.7, 1.9 Hz, 1H), 6.13 (t, J=6.2 Hz, 1H), 4.90 (s, 1H), 3.33-3.31 (m, 1H), 3.20-3.17 (m, 3H), 2.51-2.50 (m, 1H), 2.49-2.46 (m, 1H), 2.42-2.28 (m, 2H), 1.74-1.45 (m, 5H), 1.43-1.34 (m, 5H).

### Example 9: Compound 107 and Compound 108—rel-(2R,5S)-7-oxo-6-oxa-8,14,20,22,25-pentaazatetracyclo[19.2.2.1\hat{ }{2,5}.1\hat{ }{15,19}]heptacosa-1(23),15,17,19(26),21,24-hexaene-16-sulfonamide & rel-(2S,5R)-7-oxo-6-oxa-8,14,20,22,25-pentaazatetracyclo[19.2.2.1\hat{ }{2,5}.1\hat{ }{15,19}]heptacosa-1(23),15,17,19(26),21,24-hexaene-16-sulfonamide

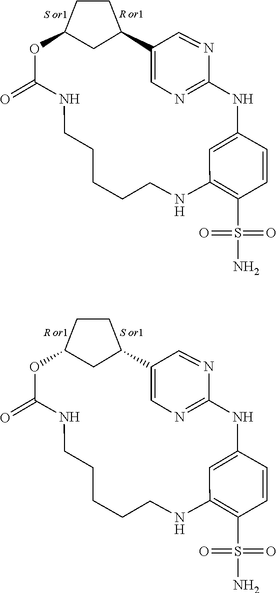 [SMILES: NS(=O)(=O)c1ccc2cc1NCCCCCNC(=O)O[C@H]1CC[C@H](C1)c1cnc(nc1)N2 ; NS(=O)(=O)c1ccc2cc1NCCCCCNC(=O)O[C@@H]1CC[C@@H](C1)c1cnc(nc1)N2]

### Step 1: Preparation of (Isomer 1) rel-(2R,5S)-7-oxo-6-oxa-8,14,20,22,25-pentaazatetracyclo[19.2.2.1\hat{ }{2,5}.1\hat{ }{15,19}]heptacosa-1(23),15,17,19(26),21,24-hexaene-16-sulfonamide & (Isomer 2) rel-(2S,5R)-7-oxo-6-oxa-8,14,20,22,25-pentaazatetracyclo[19.2.2.1\hat{ }{2,5}.1\hat{ }{15,19}]heptacosa-1(23),15,17,19(26),21,24-hexaene-16-sulfonamide

(cis)-7-oxo-6-oxa-8,14,20,22,25-pentaazatetracyclo[19.2.2.1\hat{ }{2,5}.1\hat{ }{15,19}]heptacosa-1 (23),15,17,19(26),21,24-hexaene-16-sulfonamide (37 mg) was isolated by prep-Chiral-HPLC with the following conditions: Column: CHIRALPAK IH 3*25 cm, 5 μm; Mobile Phase A: Hex (10 mM NH₃-MeOH), Mobile Phase B: EtOH; Flow rate: 40 mL/min; Gradient: isocratic 50; Wave Length: 288/204 nm; RT1 (min): 14.46; RT2 (min): 21.7. The pure fraction was concentrated under reduced pressure and by lyophilization to afford (isomer 1) rel-(2R,5S)-7-oxo-6-oxa-8,14,20,22,25-pentaazatetracyclo[19.2.2.1\hat{ }{2,5}.1\hat{ }{15,19}]heptacosa-1 (23),15,17,19(26),21,24-hexaene-16-sulfonamide (8.3 mg, 22.23%) as an off-white solid with 100% ee and (isomer 2) rel-(2S,5R)-7-oxo-6-oxa-8,14,20,22,25-pentaazatetracyclo[19.2.2.1\hat{ }{2,5}.1\hat{ }{15,19}]heptacosa-1(23),15,17,19(26),21,24-hexaene-16-sulfonamide (8.1 mg, 21.67%) as an off-white solid with 100% ee.

Compound 107: LC-MS: (ES+H, m/z): [M+H]⁺=461.15. ¹H NMR (400 MHz, DMSO-d₆) δ 9.80 (s, 1H), 8.51-8.31 (m, 3H), 7.48 (dd, J=9.6, 3.4 Hz, 2H), 7.10 (s, 2H), 6.48 (dd, J=8.7, 1.9 Hz, 1H), 6.19 (t, J=6.1 Hz, 1H), 4.95 (d, J=4.4 Hz, 1H), 3.41-3.38 (m, 1H), 3.25-3.23 (m, 3H), 2.72-2.69 (m, 1H), 2.40-2.31 (m, 2H), 1.99-1.78 (m, 5H), 1.58-1.39 (m, 5H).

Compound 108: LC-MS: (ES+H, m/z): [M+H]⁺=461.10. ¹H NMR (400 MHz, DMSO-d₆) δ 9.80 (s, 1H), 8.51-8.31 (m, 3H), 7.47 (dd, J=9.6, 3.4 Hz, 2H), 7.10 (s, 2H), 6.47 (dd, J=8.7, 1.9 Hz, 1H), 6.18 (t, J=6.1 Hz, 1H), 4.95 (d, J=4.4 Hz, 1H), 3.48-3.40 (m, 1H), 3.25-3.23 (m, 3H), 2.92-2.79 (m, 1H), 2.40-2.13 (m, 2H), 1.91-1.78 (m, 5H), 1.53-1.29 (m, 5H).

### Example 10: Compound 109—1⁵-bromo-5¹-methyl-5¹H-13-oxa-4-thia-2,7-diaza-1(2,4)-pyrimidina-3(4,1)-piperidina-5(4,5)-pyrazolacyclotridecaphan-6-one 4,4-dioxide

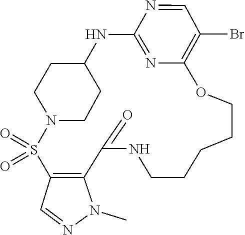 [SMILES: Cn1ncc2c1C(=O)NCCCCCOc1nc(ncc1Br)NC1CCN(CC1)S2(=O)=O]

### Step 1: Preparation of methyl 4-((4-((tert-butoxycarbonyl)amino)piperidin-1-yl)sulfonyl)-1-methyl-1H-pyrazole-5-carboxylate

To a stirring mixture of tert-butyl N-(piperidin-4-yl)carbamate (252 mg, 1.2 eq., 1.26 mmol) and ethylbis(propan-2-yl)amine (338 mg, 2.5 eq., 2.62 mmol) in dichloromethane (10.8 mL, 169 mmol) which was cooled in an ice-bath, was added methyl 4-(chlorosulfonyl)-1-methyl-1H-pyrazole-5-carboxylate (250 mg, 1.05 mmol) in one portion. Reaction mixture was then stirred for 30 min while it was slowly warmed to room temperature. After confirming a good conversion with LCMS, reaction mixture was quenched with MeOH (1 mL) then directly subjected to a flash column chromatography (0 to 100% EtOAc in hexanes) to afford methyl 4-((4-((tert-butoxycarbonyl)amino)piperidin-1-yl)sulfonyl)-1-methyl-1H-pyrazole-5-carboxylate (350 mg, 83%) as a colorless oil. LC-MS: (ES+H, m/z) 403.1 [M+H]⁺.

### Step 2: Synthesis of 4-((4-((tert-butoxycarbonyl)amino)piperidin-1-yl)sulfonyl)-1-methyl-1H-pyrazole-5-carboxylic Acid

To a stirring suspension of methyl 4-[(4-{[(tert-butoxy)carbonyl]amino}piperidin-1-yl)sulfonyl]-1-methyl-1H-pyrazole-5-carboxylate (422 mg, 1.05 mmol) in 1,4-dioxane (10 mL, 117 mmol)/water (10 mL, 555 mmol), was added 2N—NaOH solution (1 mL, 1.95 eq). The resulting suspension was then stirred for 2 h at room temperature. After confirming a good conversion by LCMS, 0.5N HCl (aq.) was added to quench the reaction. Organic solvent was removed then the product was extracted with EtOAc (10 mL×3). Combined organic layer was washed, dried over MgSO₄ and concentrated to afford 4-((4-((tert-butoxycarbonyl)amino)piperidin-1-yl)sulfonyl)-1-methyl-1H-pyrazole-5-carboxylic acid as a relatively pure form. The crude material was used directly without further purification. LC-MS: (ES+H, m/z) 389.1 [M+H]⁺.

### Step 3: Synthesis of tert-butyl (1-((5-((5-hydroxypentyl)carbamoyl)-1-methyl-1H-pyrazol-4-yl)sulfonyl)piperidin-4-yl)carbamate

To a stirring mixture of 4-[(4-{[(tert-butoxy)carbonyl]amino}piperidin-1-yl)sulfonyl]-1-methyl-1H-pyrazole-5-carboxylic acid (300 g, 772 mmol), 5-aminopentan-1-ol (95.6 g, 1.2 eq., 927 mmol), and hexafluoro-λ⁵-phosphamide 1-[bis(dimethylamino)methylidene]-1H-1λ⁵-[1,2,3]triazolo[4,5-b]pyridin-3-ium-1-ylium-3-olate (352 g, 1.2 eq., 927 mmol) in dimethylformamide, was added ethylbis(propan-2-yl)amine (120 g, 1.2 eq., 927 mmol) in one portion at room temperature. The suspension was stirred for 30 min then solvent was removed via a speed-vac (Genevac). The crude material was then subjected to a flash column chromatography (0 to 100% EtOAc in hexanes) to afford tert-butyl (1-((5-((5-hydroxypentyl)carbamoyl)-1-methyl-1H-pyrazol-4-yl)sulfonyl)piperidin-4-yl)carbamate (260 mg, 71%) as a white solid. LC-MS: (ES+H, m/z) 496.3 [M+Na]⁺.

### Step 4: Synthesis of tert-butyl (1-((5-((5-((5-bromo-2-chloropyrimidin-4-yl)oxy)pentyl)carbamoyl)-1-methyl-1H-pyrazol-4-yl)sulfonyl)piperidin-4-yl)carbamate

To a stirring suspension of tert-butyl N-[1-({5-[(5-hydroxypentyl)carbamoyl]-1-methyl-1H-pyrazol-4-yl}sulfonyl)piperidin-4-yl]carbamate (0.1 g, 211 μmol) in tetrahydrofuran (5 mL, 61.4 mmol) dipped in an ice-bath, was added sodium hydride, 60% in mineral oil (1.1 eq., 232 μmol). Resulting suspension was stirred while it was warmed to room temperature for 30 min. Thereafter, 5-bromo-2,4-dichloropyrimidine (72.2 mg, 1.5 eq., 317 μmol) was added and stirred for 15 h. The crude material was then subjected to a flash column chromatography (0 to 100% EtOAc in hexanes) to afford tert-butyl (1-((5-((5-((5-bromo-2-chloropyrimidin-4-yl)oxy)pentyl)carbamoyl)-1-methyl-1H-pyrazol-4-yl)sulfonyl)piperidin-4-yl)carbamate (90 mg, 64%). LC-MS: (ES+H, m/z) 664.2 [M+H]⁺.

### Step 5: Synthesis of 4-((4-aminopiperidin-1-yl)sulfonyl)-N-(5-((5-bromo-2-chloropyrimidin-4-yl)oxy)pentyl)-1-methyl-1H-pyrazole-5-carboxamide

To a 100 mL round bottom flask charged with a stir bar, was added tert-butyl N-(1-{[5-({5-[(5-bromo-2-chloropyrimidin-4-yl)oxy]pentyl}carbamoyl)-1-methyl-1H-pyrazol-4-yl]sulfonyl}piperidin-4-yl)carbamate (80 mg, 120 μmol), triethylsilane, formic acid (35 mL, 920 mmol) and dichloromethane (35 mL, 547 mmol). The reaction mixture was then stirred at 36° C. for 14 h. The crude material was filtered then directly subjected to a flash column chromatography (C-18 column; 10 to 100% Acetonitrile in Water, 5 μmol HCl as an modifier) to afford 4-((4-aminopiperidin-1-yl)sulfonyl)-N-(5-((5-bromo-2-chloropyrimidin-4-yl)oxy)pentyl)-1-methyl-1H-pyrazole-5-carboxamide (48 mg, 60%). LC-MS: (ES+H, m/z) 564.2 [M+H]⁺.

### Step 6: Synthesis of 1⁵-bromo-5¹-methyl-5¹H-13-oxa-4-thia-2,7-diaza-1(2,4)-pyrimidina-3(4,1)-piperidina-5(4,5)-pyrazolacyclotridecaphan-6-one 4,4-dioxide

To a microwave vial (Biotage, 0.5 mL-2 mL) charged with a stir bar, was added 4-[(4-aminopiperidin-1-yl)sulfonyl]-N-{5-[(5-bromo-2-chloropyrimidin-4-yl)oxy]pentyl}-1-methyl-1H-pyrazole-5-carboxamide (10 mg, 17.7 μmol), dipotassium carbonate (12.2 mg, 5 eq., 88.5 μmol) and dimethyl sulfoxide (2.94 mL, 41.1 mmol). After capping, the microwave vial was then capped and subjected to a microwave run at 110° C. for 2 h. The crude material was filtered then directly subjected to a flash column chromatography (0 to 100% EtOAc in hexanes) to afford 1⁵-bromo-5¹-methyl-5¹H-13-oxa-4-thia-2,7-diaza-1(2,4)-pyrimidina-3(4,1)-piperidina-5 (4,5)-pyrazolacyclotridecaphan-6-one 4,4-dioxide (3.1 mg, 33%) as a white solid. LC-MS: (ES+H, m/z) 528.2 [M+H]⁺; ¹H NMR (400 MHz, DMSO) δ 8.83 (t, J=6.0 Hz, 1H), 8.15 (s, 1H), 7.74 (s, 1H), 7.49 (d, J=7.4 Hz, 1H), 4.44-4.32 (m, 2H), 3.85 (s, 3H), 3.58 (d, J=11.4 Hz, 2H), 3.53-3.44 (m, 2H), 3.38-3.37 (m, 1H), 2.02 (d, J=12.6 Hz, 2H), 1.74-1.62 (m, 2H), 1.55-1.22 (m, 8H).

### Biochemical Assays

### CDK2/CyclinA2 ChEF Assay

500 nL of 50× compound in 100% DMSO is added to a 384-well low volume, white assay plate. 19.5 μL of 1.3×CDK2/CyclinA2 (Wild-type, full length CDK2 in complex with wildtype, full length CyclinA2: Carna Cat #04-103 diluted in assay buffer (50 mM Hepes, 0.01% Brij-35, 10 mM MgCl2, 0.5 mM EGTA, 1% Glycerol, and 1.2 mM DTT) is added to the plates on top of the compound and pre-incubated for 30 minutes at room temperature. Following the incubation, 5 μL of 5×Sox-chromophore (CSox) labeled peptide substrate (AQT0255, AssayQuant) & ATP diluted in assay buffer is added to the plate to initiate the reaction. The assay is incubated at room temperature for 45 min after initiation. The CSox peptide substrate is phosphorylated by CDK2/A2 upon ATP hydrolysis.

### CDK1/CyclinB1 ChEF Assay

500 nL of 50× compound in 100% DMSO is added to a 384-well low volume, white assay plate. 19.5 μL of 1.3×CDK1/CyclinB1 (Wild-type, full length CDK1 in complex with wildtype, full length CyclinB1: Carna Cat #04-102) diluted in assay buffer (50 mM Hepes, 0.01% Brij-35, 10 mM MgCl2, 0.5 mM EGTA, 1% Glycerol, and 1.2 mM DTT) is added to the plates on top of the compound and pre-incubated for 30 minutes at room temperature. Following the incubation, 5 μL of 5×Sox-chromophore (CSox) labeled peptide substrate (AQT0297, AssayQuant) & ATP diluted in assay buffer is added to the plate to initiate the reaction. The assay is incubated at room temperature for 30 min after initiation. The CSox peptide substrate is phosphorylated by CDK1/B1 upon ATP hydrolysis.

### CDK9/CyclinT1 ChEF Assay

500 nL of 50× compound in 100% DMSO is added to a 384-well low volume, white assay plate. 19.5 μL of 1.3×CDK9/CyclinT1 (Wild-type, full length CDK9 in complex with wildtype, full length CyclinT1: Invitrogen Cat #PV4131) diluted in assay buffer (50 mM Hepes, 0.01% Brij-35, 10 mM MgCl2, 0.5 mM EGTA, 1% Glycerol, and 1.2 mM DTT) is added to the plates on top of the compound and pre-incubated for 30 minutes at room temperature. Following the incubation, 5 μL of 5×Sox-chromophore (CSox) labeled peptide substrate (AQT0449, AssayQuant) & ATP diluted in assay buffer is added to the plate to initiate the reaction. The assay is incubated at room temperature for 120 min after initiation. The CSox peptide substrate is phosphorylated by CDK9/T1 upon ATP hydrolysis.

The fluorescent intensity measurement of the assay plate for all assays described above is read on a microplate reader once the assay is initiated and again at the completion of the assay. Phosphorylation of the CSox-based peptide substrate sensor results in an immediate increase in fluorescence. These raw fluorescence intensity values are normalized to calculate percent inhibition, which are fit to a four-parameter logistic curve and IC50 values are calculated. IC50 values are converted to Ki using substrate, K_{M}^{app} values, and the Cheng-Prusoff equation.

Table A lists the following.
CDK2/CCNA2 ChEF 10×Km Ki: Average Ki (nM) (A<1 nM; 1 nM≤B<25 nM; 25 nM≤C<100 nM; D≥100 nM)
CDK2/CCNA2 ChEF 10×Km Ki: CDK2/A2 selectivity over CDK1/B1 (A>10; 5≤B<10; C<5)
CDK2/CCNA2 ChEF 10×Km Ki: CDK2/A2 Selectivity over CDK9/T1 (A>10; 5≤B<10; C<5)
CDK1/CCNB1 ChEF Ki: Average Ki (nM) (A<1 nM; 1 nM≤B<25 nM; 25 nM≤C<100 nM; D≥100 nM)
CDK9/CCNT1 ChEF 10×Km Ki: Average Ki (nM) (A<1 nM; 1 nM≤B<25 nM; 25 nM≤C<100 nM; D≥100 nM)
ND=not determined

TABLE A
Biological Activity of Selected Compounds

| | | CDK2/CCN | CDK2/CCN | | |
|---|---|---|---|---|---|
| | CDK2/CCN | A2 ChEF | A2 ChEF | | CDK9/CCN |
| | A2 ChEF | 10xKm Ki: | 10xKm Ki: | CDK1/CCN | T1 ChEF |
| | 10xKm Ki: | CDK2/A2 | CDK2/A2 | B1 ChEF Ki: | 10xKm Ki: |
| Compound | Average Ki | selectivity | Selectivity | Average Ki | Average Ki |
| No. | (nM) | over CDK1/B1 | over CDK9/T1 | (nM) | (nM) |
| 89 | B | B | A | B | D |
| 90 | B | B | A | B | D |
| 91 | A | D | A | B | D |
| 92 | A | B | A | A | D |
| 94 | C | C | A | D | B |
| 95 | D | B | C | D | D |
| 96 | B | B | A | C | C |
| 97 | A | D | A | B | D |
| 98 | C | C | B | D | C |
| 99 | A | D | A | B | D |
| 100 | B | B | A | C | D |
| 101 | B | B | A | C | C |
| 102 | A | D | A | B | D |
| 103 | ND | ND | ND | ND | ND |
| 104 | ND | ND | ND | ND | ND |
| 105 | ND | ND | ND | ND | ND |
| 111 | ND | ND | ND | ND | ND |
| 112 | ND | ND | ND | ND | ND |
| 113 | ND | ND | ND | ND | ND |
| 114 | ND | ND | ND | ND | ND |
| 115 | ND | ND | ND | ND | ND |

### CDK1/Cyclin B1 Mobility Shift Assay

10 nL of 2000× compound in 100% DMSO is added to a 384-well polypropylene assay plate. 15 μL of 1.3×CDK1/B1 and 1.3× fluorescent substrate (Wild-type, full length CDK1 in complex with wildtype, full length CyclinB1: Carna Cat #04-102 and FL-Peptide 29: PerkinElmer Cat #760429) diluted in kinase buffer (50 mM Hepes, 0.01% Brij-35, 10 mM MgCl2, 1 mM EGTA, 0.05% BSA, and 2 mM DTT) is added to the plates on top of the compound, mixed, and pre-incubated for 30 minutes at room temperature. The fluorescently labeled peptide substrate is phosphorylated by CDK1/B1 upon ATP hydrolysis. Following the incubation, 5 μL of 4×ATP diluted in kinase buffer is added to the plate to initiate the reaction. Following the 30-minute incubation, the reaction is stopped by adding 75 μL of stopping buffer containing 0.5 M EDTA.

The assay plate is then read on a Lab Chip EZ Reader which separates the fluorescently labeled substrate and product peptides through a mobility difference. The phosphorylation of the substrate imparts a negative charge, which allows differences in separation as both are pulled through the microfluidic chip. The ratio of substrate and product values are used to generate percent conversion. These raw percent conversion values are normalized to calculate percent inhibition, which are fit to a four-parameter logistic curve and IC₅₀ values are calculated. IC₅₀ values are converted to Ki using substrate, Kmapp values, and the Cheng-Prusoff equation.

### CDK9/CyclinT1 Mobility Shift Assay

10 nL of 2000× compound in 100% DMSO is added to a 384-well polypropylene assay plate. 15 μL of 1.3×CDK9/T1 and 1.3× fluorescent substrate (Wild-type, full length CDK9 in complex with wildtype, CyclinT1 (1-259): Biortus Cat #BP480/792/691 and FL-Peptide 34: PerkinElmer Cat #760643) diluted in kinase buffer (50 mM Hepes, 0.01% Brij-35, 10 mM MgCl₂, 1 mM EGTA, 0.05% BSA, and 2 mM DTT) is added to the plates on top of the compound, mixed, and pre-incubated for 30 minutes at room temperature. The fluorescently labeled peptide substrate is phosphorylated by CDK9/T1 upon ATP hydrolysis. Following the incubation, 5 μL of 4×ATP diluted in kinase buffer is added to the plate to initiate the reaction. Following the 30-minute incubation, the reaction is stopped by adding 75 μL of stopping buffer containing 0.5 M EDTA.

The assay plate is then read on a Lab Chip EZ Reader which separates the fluorescently labeled substrate and product peptides through a mobility difference. The phosphorylation of the substrate imparts a negative charge, which allows differences in separation as both are pulled through the microfluidic chip. The ratio of substrate and product values are used to generate percent conversion. These raw percent conversion values are normalized to calculate percent inhibition, which are fit to a four-parameter logistic curve and IC50 values are calculated. IC50 values are converted to Ki using substrate, Kmapp values, and the Cheng-Prusoff equation.

### CDK2/Cyclin A2 Mobility Shift Assay

10 nL of 2000× compound in 100% DMSO is added to a 384-well polypropylene assay plate. 15 μL of 1.3×CDK2/A2 and 1.3× fluorescent substrate (Wild-type, full length CDK2 in complex with wildtype, full length CyclinA2: Carna Cat #04-103 and FL-Peptide 18: PerkinElmer Cat #760362) diluted in kinase buffer (50 mM Hepes, 0.01% Brij-35, 10 mM MgCl2, 1 mM EGTA, 0.05% BSA, and 2 mM DTT) is added to the plates on top of the compound, mixed, and pre-incubated for 30 minutes at room temperature. The fluorescently labeled peptide substrate is phosphorylated by CDK2/A2 upon ATP hydrolysis. Following the incubation, 5 μL of 4×ATP diluted in kinase buffer is added to the plate to initiate the reaction. Following the 30-minute incubation, the reaction is stopped by adding 75 μL of stopping buffer containing 0.5 M EDTA.

The assay plate is then read on a Lab Chip EZ Reader which separates the fluorescently labeled substrate and product peptides through a mobility difference. The phosphorylation of the substrate imparts a negative charge, which allows differences in separation as both are pulled through the microfluidic chip. The ratio of substrate and product values are used to generate percent conversion. These raw percent conversion values are normalized to calculate percent inhibition, which are fit to a four-parameter logistic curve and IC₅₀ values are calculated. IC₅₀ values are converted to Ki through using substrate and Kₘ^{app} values, and the Cheng-Prusoff equation.

### CDK6/Cyclin D3 Mobility Shift Assay

10 nL of 2000× compound in 100% DMSO is added to a 384-well polypropylene assay plate. 15 μL of 1.3×CDK6/D3 and 1.3× fluorescent substrate (Wild-type, full length CDK6 in complex with wildtype, full length CyclinD3: Carna Cat #04-107 and FL-Peptide 34: PerkinElmer Cat #760643) diluted in kinase buffer (50 mM Hepes, 0.01% Brij-35, 10 mM MgCl2, 1 mM EGTA, 0.05% BSA, and 2 mM DTT) is added to the plates on top of the compound, mixed, and pre-incubated for 30 minutes at room temperature. The fluorescently labeled peptide substrate is phosphorylated by CDK6/D3 upon ATP hydrolysis. Following the incubation, 5 μL of 4×ATP diluted in kinase buffer is added to the plate to initiate the reaction. Following the 30-minute incubation, the reaction is stopped by adding 75 μL of stopping buffer containing 0.5 M EDTA.

The assay plate is then read on a Lab Chip EZ Reader which separates the fluorescently labeled substrate and product peptides through a mobility difference. The phosphorylation of the substrate imparts a negative charge, which allows differences in separation as both are pulled through the microfluidic chip. The ratio of substrate and product values are used to generate percent conversion. These raw percent conversion values are normalized to calculate percent inhibition, which are fit to a four-parameter logistic curve and IC₅₀ values are calculated. IC₅₀ values are converted to Ki through using substrate and Kₘ^{app} values, and the Cheng-Prusoff equation.

Table B lists the following.
CDK2/CCNA2 Caliper 10×Km Ki: CDK2/A2 selectivity over CDK1/B1 (A>10; 5≤B<10; C<5)
CDK2/CCNA2 Caliper 10×Km Ki: CDK2/A2 selectivity over CDK6/D3 (A>10; 5≤B<10; C<5)
CDK2/CCNA2 Caliper 10×Km Ki: CDK2/A2 selectivity over CDK9 (A>10; 5≤B<10; C<5)
CDK2/CCNA2 Caliper 10×Km Ki: Average Ki (A<1 nM; 1 nM≤B<25 nM; 25 nM≤C<100 nM; D≥100 nM)
CDK1/CCNB1 Caliper Ki (A<1 nM; 1 nM≤B<25 nM; 25 nM≤C<100 nM; D≥100 nM
CDK6/CCND3 Caliper Ki: Average Ki (A<1 nM; 1 nM≤B<25 nM; 25 nM≤C<100 nM; D≥100 nM)
CDK9/CyclinT1 Caliper Ki: Average Ki (A<1 nM; 1 nM≤B<25 nM; 25 nM≤C<100 nM; D≥100 nM)
ND=not determined

TABLE B
Biological Activity of Selected Compounds

| | | CDK2/CCNA2 | CDK2/CCNA2 | CDK2/CCNA2 | | | |
|---|---|---|---|---|---|---|---|
| | CDK2/CCNA2 | Caliper | Caliper | Caliper | CDK1/C | CDK6/C | CDK9/Cyc |
| | Caliper | 10xKm Ki: | 10xKm Ki: | 10xKm Ki: | CNB1 | CND3 | lin T1 |
| | 10xKm | CDK2/A2 | CDK2/A2 | CDK2/A2 | Caliper | Caliper | Caliper |
| | Ki: | Selectivity | Selectivity | Selectivity | Ki: | Ki: | Ki: |
| Compound | Average | over | over | over | Average | Average | Average |
| No. | Ki (nM) | CDK1/B1 | CDK6/D3 | CDK9 | Ki (nM) | Ki (nM) | Ki (nM) |
| 89 | B | A | ND | A | B | ND | B |
| 90 | B | A | ND | A | C | ND | C |
| 91 | A | A | A | A | B | D | C |
| 92 | A | A | A | A | A | C | C |
| 94 | ND | ND | ND | ND | ND | ND | C |
| 95 | C | B | ND | B | D | ND | D |
| 96 | B | A | ND | A | D | ND | D |
| 97 | B | A | ND | A | B | ND | D |
| 98 | C | A | ND | A | D | ND | D |
| 99 | A | A | A | A | B | C | D |
| 100 | B | A | ND | A | D | ND | D |
| 101 | B | A | ND | A | D | ND | D |
| 102 | A | A | A | A | C | D | D |
| 103 | ND | ND | ND | ND | ND | ND | D |
| 104 | ND | ND | ND | ND | ND | ND | D |
| 105 | ND | ND | ND | ND | ND | ND | D |
| 111 | C | C | ND | B | D | ND | D |
| 112 | C | B | ND | B | D | ND | D |
| 113 | D | B | ND | B | D | ND | ND |
| 114 | C | B | ND | B | D | ND | ND |
| 115 | D | B | ND | C | D | ND | ND |

### CDK2/CyclinA2 ChEF Assay

16 nL of 1000× compound in 100% DMSO is added to a 384-well low volume, black assay plate. 12 μL of 1.3×CDK2/CyclinA2 (Wild-type, full length CDK2 in complex with wildtype, full length CyclinA2: Carna Cat #04-103 diluted in assay buffer (54 mM Hepes, 0.012% Brij-35, 10 mM MgCl2, 0.55 mM EGTA, 1% Glycerol, 0.02% BSA, and 1.2 mM DTT) is added to the plates on top of the compound and pre-incubated for 30 minutes at room temperature. Following the incubation, 4 uL of 4×Sox-chromophore (CSox) labeled peptide substrate (AQT0297, AssayQuant) & ATP diluted in assay buffer is added to the plate to initiate the reaction. The assay is incubated at room temperature for 90 min after initiation. The CSox peptide substrate is phosphorylated by CDK2/A2 upon ATP hydrolysis.

The fluorescent intensity measurement of the assay plate is read on a microplate reader once the assay is initiated and again at the completion of the assay. Phosphorylation of the CSox-based peptide substrate sensor results in an immediate increase in fluorescence. These raw fluorescence intensity values are normalized to calculate percent inhibition, which are fit to a four-parameter logistic curve and IC₅₀ values are calculated. IC₅₀ values are converted to Ki using substrate, K_{M}^{app} values, and the Cheng-Prusoff equation.

### CDK6/CyclinD3 ChEF Assay

16 nL of 1000× compound in 100% DMSO is added to a 384-well low volume, black assay plate. 12 μL of 1.3×CDK6/CyclinD3 (Wild-type, full length CDK6 in complex with wildtype, full length CyclinD3: Carna Cat #04-107) diluted in assay buffer (54 mM Hepes, 0.012% Brij-35, 10 mM MgCl2, 0.55 mM EGTA, 1% Glycerol, 0.02% BSA, and 1.2 mM DTT) is added to the plates on top of the compound and pre-incubated for 30 minutes at room temperature. Following the incubation, 4 uL of 4×Sox-chromophore (CSox) labeled peptide substrate (AQT0258, AssayQuant) & ATP diluted in assay buffer is added to the plate to initiate the reaction. The assay is incubated at room temperature for 300 min after initiation. The CSox peptide substrate is phosphorylated by CDK6/D3 upon ATP hydrolysis.

The fluorescent intensity measurement of the assay plate is read on a microplate reader once the assay is initiated and again at the completion of the assay. Phosphorylation of the CSox-based peptide substrate sensor results in an immediate increase in fluorescence. These raw fluorescence intensity values are normalized to calculate percent inhibition, which are fit to a four-parameter logistic curve and IC₅₀ values are calculated. IC₅₀ values are converted to Ki using substrate, K_{M}^{app} values, and the Cheng-Prusoff equation.

### CDK1/CyclinB1 ChEF Assay

16 nL of 1000× compound in 100% DMSO is added to a 384-well low volume, black assay plate. 12 μL of 1.3×CDK1/CyclinB1 (Wild-type, full length CDK1 in complex with wildtype, full length CyclinB1: Carna Cat #04-102) diluted in assay buffer (54 mM Hepes, 0.012% Brij-35, 10 mM MgCl2, 0.55 mM EGTA, 1% Glycerol, 0.02% BSA, and 1.2 mM DTT) is added to the plates on top of the compound and pre-incubated for 30 minutes at room temperature. Following the incubation, 4 uL of 4×Sox-chromophore (CSox) labeled peptide substrate (AQT0297, AssayQuant) & ATP diluted in assay buffer is added to the plate to initiate the reaction. The assay is incubated at room temperature for 120 min after initiation. The CSox peptide substrate is phosphorylated by CDK1/B1 upon ATP hydrolysis.

The fluorescent intensity measurement of the assay plate is read on a microplate reader once the assay is initiated and again at the completion of the assay. Phosphorylation of the CSox-based peptide substrate sensor results in an immediate increase in fluorescence. These raw fluorescence intensity values are normalized to calculate percent inhibition, which are fit to a four-parameter logistic curve and IC₅₀ values are calculated. IC₅₀ values are converted to Ki using substrate, K_{M}^{app} values, and the Cheng-Prusoff equation.

### CDK9/CyclinT1 ChEF Assay

16 nL of 1000× compound in 100% DMSO is added to a 384-well low volume, black assay plate. 12 μL of 1.3×CDK9/CyclinT1 (Wild-type, full length CDK9 in complex with wildtype, CyclinT1 (1-259): Biortus Cat #BP480/792/691) diluted in assay buffer (54 mM Hepes, 0.012% Brij-35, 10 mM MgCl2, 0.55 mM EGTA, 1% Glycerol, 0.02% BSA, and 1.2 mM DTT) is added to the plates on top of the compound and pre-incubated for 30 minutes at room temperature. Following the incubation, 4 μL of 4×Sox-chromophore (CSox) labeled peptide substrate (AQT0449, AssayQuant) & ATP diluted in assay buffer is added to the plate to initiate the reaction. The assay is incubated at room temperature for 180 min after initiation. The CSox peptide substrate is phosphorylated by CDK9/T1 upon ATP hydrolysis.

The fluorescent intensity measurement of the assay plate is read on a microplate reader once the assay is initiated and again at the completion of the assay. Phosphorylation of the CSox-based peptide substrate sensor results in an immediate increase in fluorescence. These raw fluorescence intensity values are normalized to calculate percent inhibition, which are fit to a four-parameter logistic curve and IC₅₀ values are calculated. IC₅₀ values are converted to Ki using substrate, K_{M}^{app} values, and the Cheng-Prusoff equation.

### CDK4/CyclinD1 ChEF Assay

16 nL of 1000× compound in 100% DMSO is added to a 384-well low volume, black assay plate. 12 μL of 1.3×CDK4/CyclinD1 (Wild-type, full length CDK4 in complex with wildtype, CyclinD1: ThermoFisher Cat #PR8064A) diluted in assay buffer (54 mM Hepes, 0.012% Brij-35, 10 mM MgCl2, 0.55 mM EGTA, 1% Glycerol, 0.02% BSA, and 1.2 mM DTT) is added to the plates on top of the compound and pre-incubated for 30 minutes at room temperature. Following the incubation, 4 uL of 4×Sox-chromophore (CSox) labeled peptide substrate (AQT0258, AssayQuant) & ATP diluted in assay buffer is added to the plate to initiate the reaction. The assay is incubated at room temperature for 150 min after initiation. The CSox peptide substrate is phosphorylated by CDK4/D1 upon ATP hydrolysis.

The fluorescent intensity measurement of the assay plate is read on a microplate reader once the assay is initiated and again at the completion of the assay. Phosphorylation of the CSox-based peptide substrate sensor results in an immediate increase in fluorescence. These raw fluorescence intensity values are normalized to calculate percent inhibition, which are fit to a four-parameter logistic curve and IC₅₀ values are calculated. IC₅₀ values are converted to Ki using substrate, K_{M}^{ap} values, and the Cheng-Prusoff equation.

Table C lists the following.
CDK2/CCNA2 AQT10×Km Ki: CDK2/A2 selectivity over CDK1/B1 (A>10; 5≤B<10; C<5)
CDK2/CCNA2 AQT10×Km Ki: CDK2/A2 selectivity over CDK4/D1 (A>10; 5≤B<10; C<5)
CDK2/CCNA2 AQT10×Km Ki: CDK2/A2 selectivity over CDK6/D3 (A>10; 5≤B<10; C<5)
CDK2/CCNA2 AQT10×Km Ki: CDK2/A2 selectivity over CDK9 (A>10; 5≤B<10; C<5)
CDK2/CCNA2 AQT10×Km Ki: Average Ki (A<1 nM; 1 nM≤B<25 nM; 25 nM≤C<100 nM; D≥100 nM)
CDK1/CCNB1 AQT10×Km Ki: Average Ki (A<1 nM; 1 nM≤B<25 nM; 25 nM≤C<100 nM; D≥100 nM)
CDK4/CCNB1 AQT10×Km Ki: Average Ki (A<1 nM; 1 nM≤B<25 nM; 25 nM≤C<100 nM; D≥100 nM)
CDK6/CCND3 AQT10×Km Ki: Average Ki (A<1 nM; 1 nM≤B<25 nM; 25 nM≤C<100 nM; D≥100 nM)
CDK9/CyclinT1 AQT10×Km Ki: Average Ki (A<1 nM; 1 nM≤B<25 nM; 25 nM≤C<100 nM; D≥100 nM)
ND=not determined

TABLE C
Biological Activity of Selected Compounds

| | | CDK2/ | CDK2/ | CDK2/ | CDK2/ | | | | |
|---|---|---|---|---|---|---|---|---|---|
| | | CCNA2 | CCNA2 | CCNA2 | CCNA2 | CDK1/ | CDK4/ | CDK6/ | CDK9/ |
| | CDK2/ | AQT10xKm | AQT10xKm | AQT10xKm | AQT10xKm | CCNB1 | CCND1 | CCND3 | CyclinT1 |
| | CCNA2 | Ki: | Ki: | Ki: | Ki: | AQT | AQT | AQT | 10XKm |
| | AQT10xKm | CDK2/A2 | CDK2/A2 | CDK2/A2 | CDK2/A2 | AQT10xKm | Ki: | Ki: | AQT |
| | Ki: Average | Selectivity | selectivity | selectivity | Selectivity | Ki: | Average | Average | Ki: |
| Compound | Ki | over | over | over | over | Average | Ki | Ki | Average |
| No. | (nM) | CDK1/B1 | CDK4/D1 | CDK6/D3 | CDK9 | Ki (nM) | (nM) | (nM) | Ki (nM) |
| 91 | A | A | A | A | A | B | D | D | C |
| 97 | A | A | ND | ND | A | B | ND | ND | C |
| 103 | A | A | ND | ND | A | B | ND | ND | B |
| 104 | A | A | ND | ND | A | B | ND | ND | D |
| 105 | A | A | A | A | A | B | D | D | C |

What is claimed is:

1. A compound of Formula (I): 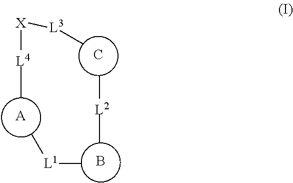
or a pharmaceutically acceptable salt thereof, wherein:
Ring A is a C3-C10 cycloalkyl optionally substituted with 1-3 independently selected R¹;
each R¹ is independently selected from halogen and C1-C6 alkyl;
Ring B is 5-10 membered heteroaryl optionally substituted with 1-3 independently selected R²;
each R² is independently selected from halogen, cyano, C1-C6 alkyl, and C1-C6 haloalkyl;
Ring C is C4-C6 cycloalkyl, 4-6 membered heterocyclyl, phenyl, or 5-6 membered heteroaryl, wherein Ring C is optionally substituted with 1-3 independently selected R³;
each R³ is independently selected from halogen, cyano, C1-C6 alkyl, C1-C6 alkoxy, C1-C6 haloalkyl, C1-C6 haloalkoxy, —NR^{A}R^{B}, —SO₂R^{A}, —NHSO₂R^{A}, and —SO₂NR^{A}R^{B},
each R^{A} and R^{B} is independently selected from hydrogen, C1-C6 alkyl, and C1-C6 haloalkyl; or
R^{A} and R^{B} together with the nitrogen atom to which they are attached form a 4-8 membered heterocyclyl optionally substituted with 1-2 substituents independently selected from halogen and C1-C6 alkyl;
L¹ is a bond;
L² is —NH— or —N(CH₃)—;
L³ is a bond, —NH—, —N(CH₃)—, —O—, *—SO₂NH—, *—NHSO₂—, *—C(═O) NH—, or *—NHC(═O)—, wherein * denotes the point of connection to Ring C;
X is a C2-C15 alkylene, a C4-C15 alkenylene, or a C5-C15 alkynylene, wherein X is optionally substituted with 1-6 independently selected RY and wherein 1-4 methylene units of X are optionally and independently replaced by —O—, —NH—, —N(C1-C6 alkyl)-, —(C3-C6 cycloalkyl)-, or -(5-6 membered heteroaryl)-;
each R^{x} is independently selected from halogen and C1-C6 alkyl;
L⁴ is a bond, *—(CH₂)ₘ—O—C(═O) NH—, or *—(CH₂)ₘ—NHC(═O) NH—, wherein * indicates the point of attachment to Ring A; and
m is 0, 1, or 2.

2. The compound of claim 1, wherein Ring A is C3-C10 cycloalkyl substituted with 1-3 independently selected R¹.

3. The compound of claim 1, wherein Ring A is an unsubstituted C3-C10 cycloalkyl.

4. The compound of any one of claims 1-3, wherein Ring A is C3-C8 cycloalkyl.

5. The compound of any one of claims 1-4, wherein Ring A is cyclobutyl, cyclopentyl, or [1,1,1]bicyclopentyl.

6. The compound of any one of claims 1-5, wherein Ring B is 5-6 membered heteroaryl substituted with 1-3 independently selected R².

7. The compound of any one of claims 1-6, wherein Ring B is an unsubstituted 5-6 membered heteroaryl.

8. The compound of any one of claims 1-7, wherein Ring B is 5 membered heteroaryl.

9. The compound of any one of claims 1-8, wherein Ring B is selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl.

10. The compound of any one of claims 1-9, wherein Ring B is pyrazolyl.

11. The compound of any one of claims 1-7, wherein Ring Bis 6 membered heteroaryl.

12. The compound of any one of claim 1-7 or 11, wherein Ring B is selected from the group consisting of pyridinyl, pyrimidinyl, pyrazinyl, pyridazinyl, and triazinyl.

13. The compound of any one of claim 1-7 or 11-12, wherein Ring B is pyrimidinyl.

14. The compound of any one of claims 1-13, wherein Ring C is C5-C6 cycloalkyl substituted with 1-3 independently selected R³.

15. The compound of any one of claims 1-13, wherein Ring C is an unsubstituted C5-C6 cycloalkyl.

16. The compound of any one of claims 1-13, wherein Ring C is 5-6 membered heterocyclyl substituted with 1-3 independently selected R³.

17. The compound of any one of claims 1-13, wherein Ring C is an unsubstituted 5-6 membered heterocyclyl.

18. The compound of any one of claim 1-13 or 16-17, wherein Ring C is 5 membered heterocyclyl.

19. The compound of any one of claim 1-13 or 16-17, wherein Ring C is 6 membered heterocyclyl.

20. The compound of any one of claim 1-13, 16-17, or 19, wherein Ring C is piperidinyl.

21. The compound of any one of claims 1-13, wherein Ring C is phenyl substituted with 1-3 independently selected R³.

22. The compound of any one of claims 1-13, wherein Ring C is an unsubstituted phenyl.

23. The compound of any one of claims 1-13, wherein Ring C is 5-6 membered heteroaryl substituted with 1-3 independently selected R³.

24. The compound of any one of claims 1-13, wherein Ring C is an unsubstituted 5-6 membered heteroaryl.

25. The compound of any one of claim 1-13 or 23-24, wherein Ring C is 5 membered heteroaryl.

26. The compound of any one of claim 1-13 or 23-25, wherein Ring C is selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl.

27. The compound of any one of claim 1-13 or 23-24, wherein Ring C is 6 membered heteroaryl.

28. The compound of any one of claim 1-13, 23-24, or 27, wherein Ring C is selected from the group consisting of pyridinyl, pyrimidinyl, pyrazinyl, pyridazinyl, and triazinyl.

29. The compound of any one of claim 1-13, 23-24, or 27-28, wherein Ring C is pyridinyl.

30. The compound of any one of claims 1-29, wherein R¹ is halogen.

31. The compound of any one of claims 1-29, wherein R¹ is C1-C6 alkyl.

32. The compound of any one of claims 1-31, wherein R² is halogen.

33. The compound of any one of claims 1-31, wherein R² is cyano.

34. The compound of any one of claims 1-31, wherein R² is C1-C6 alkyl.

35. The compound of any one of claims 1-31, wherein R² is C1-C6 haloalkyl.

36. The compound of any one of claims 1-35, wherein R³ is halogen.

37. The compound of any one of claims 1-36, wherein R³ is —F.

38. The compound of any one of claims 1-35, wherein R³ is cyano.

39. The compound of any one of claims 1-35, wherein R³ is C1-C6 alkyl.

40. The compound of any one of claims 1-35, wherein R³ is C1-C6 alkoxy.

41. The compound of any one of claims 1-35, wherein R³ is C1-C6 haloalkyl.

42. The compound of any one of claims 1-35, wherein R³ is C1-C6 haloalkoxy.

43. The compound of any one of claims 1-35, wherein R³ is —SO₂R^{A}.

44. The compound of any one of claims 1-35, wherein R³ is —NHSO₂R⁴.

45. The compound of any one of claims 1-35, wherein R³ is —NR^{A}R^{B}.

46. The compound of any one of claims 1-35, wherein R³ is —SO₂NR^{A}R^{B}.

47. The compound of any one of claim 1-35 or 43-44, wherein R^{A} is hydrogen.

48. The compound of any one of claim 1-35 or 43-44, wherein R^{A} is C1-C6 alkyl.

49. The compound of any one of claim 1-35 or 43-44, wherein R^{A} is C1-C6 haloalkyl.

50. The compound of any one of claim 1-35 or 45-46, wherein R^{A} and R^{B} are both hydrogen.

51. The compound of any one of claim 1-35 or 45-46, wherein R^{A} and R^{B} are independently selected C1-C6 alkyl.

52. The compound of any one of claim 1-35 or 45-46, wherein R^{A} and R^{B} are independently selected C1-C6 haloalkyl.

53. The compound of any one of claim 1-35 or 45-46, wherein one of R^{A} and R^{B} is hydrogen and the other one of R^{A} and R^{B} is C1-C6 alkyl.

54. The compound of any one of claim 1-35 or 45-46, wherein one of R^{A} and R^{B} is hydrogen and the other one of R^{A} and R^{B} is C1-C6 haloalkyl.

55. The compound of any one of claim 1-35 or 45-46, wherein one of R^{A} and R^{B} is C1-C6 alkyl and the other one of R^{A} and R^{B} is C1-C6 haloalkyl.

56. The compound of any one of claim 1-35 or 45-46, wherein R^{A} and R^{B} together with the nitrogen atom to which they are attached form a 4-8 membered heterocyclyl optionally substituted with 1-2 substituents independently selected from halogen and C1-C6 alkyl.

57. The compound of any one of claims 1-56, wherein L² is —NH—.

58. The compound of any one of claims 1-56, wherein L² is —N(CH₃)—.

59. The compound of any one of claims 1-58, wherein L³ is a bond.

60. The compound of any one of claims 1-58, wherein L³ is —NH—.

61. The compound of any one of claims 1-58, wherein L³ is —N(CH₃)—.

62. The compound of any one of claims 1-58, wherein L³ is —O—.

63. The compound of any one of claims 1-58, wherein L³ is *—SO₂NH—, wherein * denotes the point of connection to Ring C.

64. The compound of any one of claims 1-58, wherein L³ is *—NHSO₂—, wherein * denotes the point of connection to Ring C.

65. The compound of any one of claims 1-58, wherein L³ is *—C(═O) NH—, wherein * denotes the point of connection to Ring C.

66. The compound of any one of claims 1-58, wherein L³ is *—NHC(═O)—, wherein * denotes the point of connection to Ring C.

67. The compound of any one of claims 1-66, wherein L⁴ is a bond.

68. The compound of any one of claims 1-66, wherein L⁴ is *—(CH₂)ₘ—O—C(═O) NH—, wherein * denotes the point of connection to Ring A.

69. The compound of any one of claims 1-66, wherein L⁴ is *—(CH₂)ₘ—NHC(═O) NH—, wherein * denotes the point of connection to Ring A.

70. The compound of any one of claims 1-69, wherein X is a C2-C15 alkylene, a C4-C15 alkenylene, or a C5-C15 alkynylene, substituted with 1-6 independently selected R^{x} and wherein 1-4 methylene units of X are optionally and independently replaced by —O—, —NH—, —N(C1-C6 alkyl)-, —(C3-C6 cycloalkyl)-, or -(5-6 membered heteroaryl)-.

71. The compound of any one of claims 1-69, wherein X is a C2-C15 alkylene, a C4-C15 alkenylene, or a C5-C15 alkynylene, substituted with 1-6 independently selected R^{x} and wherein 1-4 methylene units of X are independently replaced by —O—, —NH—, —N(C1-C6 alkyl)-, —(C3-C6 cycloalkyl)-, or -(5-6 membered heteroaryl)-.

72. The compound of any one of claims 1-69, wherein X is an unsubstituted C2-C15 alkylene, an unsubstituted C4-C15 alkenylene, or an unsubstituted C5-C15 alkynylene, and wherein 1-4 methylene units of X are optionally and independently replaced by —O—, —NH—, —N(C1-C6 alkyl)-, —(C3-C6 cycloalkyl)-, or -(5-6 membered heteroaryl)-.

73. The compound of any one of claims 1-69, wherein X is an unsubstituted C2-C15 alkylene, an unsubstituted C4-C15 alkenylene, or an unsubstituted C5-C15 alkynylene, and wherein 1-4 methylene units of X are independently replaced by —O—, —NH—, —N(C1-C6 alkyl)-, —(C3-C6 cycloalkyl)-, or -(5-6 membered heteroaryl)-.

74. The compound of any one of claims 1-71, wherein X is a C2-C15 alkylene, a C4-C15 alkenylene, or a C5-C15 alkynylene, wherein X is substituted with 1-3 independently selected R^{x}.

75. The compound of any one of claims 1-71, wherein X is a C2-C15 alkylene, a C4-C15 alkenylene, or a C5-C15 alkynylene, wherein X is substituted with 2-3 independently selected R^{x} wherein two R^{x} are geminal.

76. The compound of any one of claim 1-70, 72, or 74, wherein 1-2 methylene units of X are optionally and independently replaced by —O—, —NH—, —N(C1-C6 alkyl)-, —(C3-C6 cycloalkyl)-, or -(5-6 membered heteroaryl)-.

77. The compound of any one of claims 1-75, wherein one methylene unit of X is replaced by —O— or —NH— and second methylene units of X is replaced by -(5-6 membered heteroaryl)-.

78. The compound of any one of claims 1-73, wherein one methylene unit of X is replaced by —(C3-C6 cycloalkyl)-.

79. The compound of any one of claim 1-73 or 78, wherein one methylene unit of X is replaced by

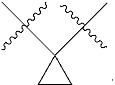 [SMILES: *C1(*)CC1]

80. The compound of any one of claims 1-79, wherein X is a C2-C15 alkylene.

81. The compound of any one of claim 1-79 or 80, wherein X is a C2-C10 alkylene.

82. The compound of any one of claim 1-79 or 80-81, wherein X is a C2-C6 alkylene.

83. The compound of any one of claims 1-82, wherein X is a C2-C4 alkylene.

84. The compound of any one of claims 1-79, wherein X is a C4-C15 alkenylene.

85. The compound of any one of claim 1-79 or 84, wherein X is a C4-C10 alkenylene.

86. The compound of any one of claim 1-79 or 84-85, wherein X is a C4-C6 alkenylene.

87. The compound of any one of claims 1-79, wherein X is a C5-C15 alkynylene.

88. The compound of any one of claim 1-79 or 87, wherein X is a C5-C10 alkynylene.

89. The compound of any one of claim 1-79 or 87-88, wherein X is a C5-C6 alkynylene.

90. The compound of any one of claims 1-89, wherein one R^{x} is halogen.

91. The compound of any one of claims 1-89, wherein one R^{x} is C1-C6 alkyl.

92. The compound of any one of claim 1-89 or 91, wherein one R^{x} is methyl.

93. The compound of any one of claims 1-92, wherein m is 0.

94. The compound of any one of claims 1-92, wherein m is 1.

95. The compound of any one of claims 1-92, wherein m is 2.

96. The compound of claim 1, wherein the compound is a compound of Formula (I-a): 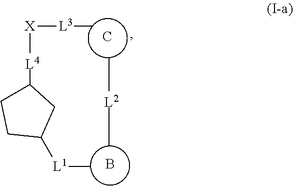
or a pharmaceutically acceptable salt thereof.

97. The compound of claim 1, wherein the compound is a compound of Formula (I-b): 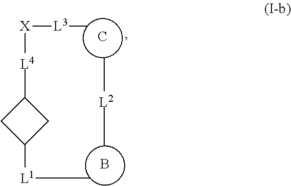
or a pharmaceutically acceptable salt thereof.

98. The compound of claim 1, wherein the compound is a compound of Formula (I-c): 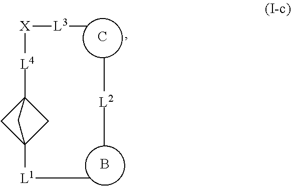
or a pharmaceutically acceptable salt thereof.

99. The compound of claim 1, wherein the compound is a compound of Formula (I-d): 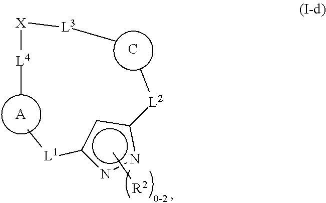
or a pharmaceutically acceptable salt thereof.

100. The compound of claim 1, wherein the compound is a compound of Formula (I-e): 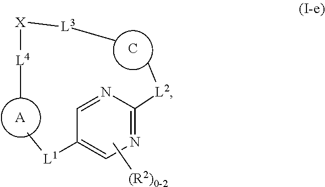
or a pharmaceutically acceptable salt thereof.

101. The compound of claim 1, wherein the compound is a compound of Formula (I-f): 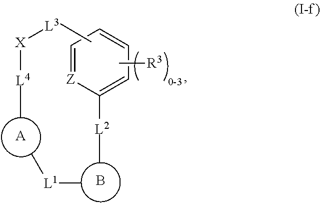
or a pharmaceutically acceptable salt thereof, wherein
Z is CH or N.

102. The compound of claim 1, wherein the compound is a compound of Formula (I-g): 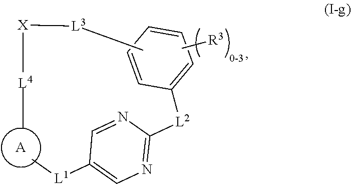
or a pharmaceutically acceptable salt thereof.

103. The compound of claim 1, wherein the compound is a compound of Formula (I-h): 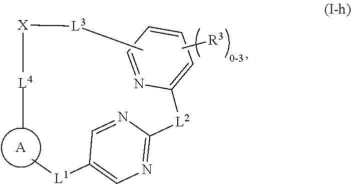
or a pharmaceutically acceptable salt thereof.

104. The compound of claim 1, wherein the compound of Formula (I) is selected from the group consisting of the compounds in Table 1, or a pharmaceutically acceptable salt thereof.

105. A pharmaceutical composition comprising a compound of any one of claims 1-104, or a pharmaceutically acceptable salt thereof, and at least one pharmaceutically acceptable excipient.

106. A method for treating cancer in a subject in need thereof, comprising administering to the subject a therapeutically effective amount of a compound of any one of claims 1-104 or a pharmaceutically acceptable salt thereof, or a pharmaceutical composition of claim 105.

107. A method for treating a cancer in a subject in need thereof, comprising:
(a) identifying the cancer as being a CDK2-associated cancer; and
(b) administering to the subject a therapeutically effective amount of a compound of any one of claims 1-104 or a pharmaceutically acceptable salt thereof, or a pharmaceutical composition of claim 105.

108. The method of claim 107, wherein the step of identifying the cancer in the subject as a CDK2-associated cancer includes performing an assay to detect dysregulation in a CDK2 gene, a CDK2 protein, or expression or activity or level of any of the same in a sample from the subject.

109. The method of claim 107, wherein the step of identifying the cancer in the subject as a CDK2-associated cancer includes performing an assay to detect dysregulation in a cyclin A2 gene, a cyclin A2 protein, or expression or activity or level of any of the same in a sample from the subject.

110. The method of claim 107, wherein the step of identifying the cancer in the subject as a CDK2-associated cancer includes performing an assay to detect dysregulation in a cyclin E1 gene, a cyclin E1 protein, or expression or activity or level of any of the same in a sample from the subject.

111. The method of claim 107, wherein the step of identifying the cancer in the subject as a CDK2-associated cancer includes performing an assay to detect dysregulation in a cyclin E2 gene, a cyclin E2 protein, or expression or activity or level of any of the same in a sample from the subject.

112. The method of any one of claims 107-111, further comprising obtaining a sample from the subject.

113. The method of claim 112, wherein the sample is a biopsy sample.

114. The method of any one of claims 108-113, wherein the assay is selected from the group consisting of sequencing, immunohistochemistry, enzyme-linked immunosorbent assay, and fluorescence in situ hybridization (FISH).

115. The method of claim 114, wherein the sequencing is pyrosequencing or next generation sequencing.

116. A method for treating a cancer in a subject in need thereof, comprising:
administering to the subject a therapeutically effective amount of a compound of any one of claims 1-104 or a pharmaceutically acceptable salt thereof, or a pharmaceutical composition of claim 105; wherein the subject has been identified as having a CDK2-associated cancer.

117. A method of treating a CDK2-associated cancer, comprising administering a therapeutically effective amount of a compound of any one of claims 1-104 or a pharmaceutically acceptable salt thereof, or a pharmaceutical composition of claim 105, to a subject identified or diagnosed as having a CDK2-associated cancer.

118. A method for treating cancer in a subject in need thereof, comprising:
(a) determining that the cancer is associated with a dysregulation of a CDK2 gene, a CDK2 protein, or expression or activity or level of any of the same; and
(b) administering to the subject a therapeutically effective amount of a compound of any one of claims 1-104 or a pharmaceutically acceptable salt thereof, or a pharmaceutical composition of claim 105.

119. The method of claim 118, wherein the step of determining that the cancer in the subject is a CDK2-associated cancer includes performing an assay to detect dysregulation in a CDK2 gene, a CDK2 protein, or expression or activity or level of any of the same in a sample from the subject.

120. The method of claim 118, wherein the step of determining that the cancer in the subject is a CDK2-associated cancer includes performing an assay to detect dysregulation in a cyclin A2 gene, a cyclin A2 protein, or expression or activity or level of any of the same in a sample from the subject.

121. The method of claim 118, wherein the step of determining that the cancer in the subject is a CDK2-associated cancer includes performing an assay to detect dysregulation in a cyclin E1 gene, a cyclin E1 protein, or expression or activity or level of any of the same in a sample from the subject.

122. The method of claim 118, wherein the step of determining that the cancer in the subject is a CDK2-associated cancer includes performing an assay to detect dysregulation in a cyclin E2 gene, a cyclin E2 protein, or expression or activity or level of any of the same in a sample from the subject.

123. The method of any one of claims 118-122, further comprising obtaining a sample from the subject.

124. The method of claim 123, wherein the sample is a biopsy sample.

125. The method of any one of claims 119-124, wherein the assay is selected from the group consisting of sequencing, immunohistochemistry, enzyme-linked immunosorbent assay, and fluorescence in situ hybridization (FISH).

126. The method of claim 125, wherein the sequencing is pyrosequencing or next generation sequencing.

127. A method for inhibiting metastasis in a subject having a cancer in need thereof, comprising administering to the subject a therapeutically effective amount of a compound of any one of claims 1-104 or a pharmaceutically acceptable salt thereof, or a pharmaceutical composition of claim 105.

128. The method of any one of claims 106-127, further comprising administering an additional therapy or therapeutic agent to the subject.

129. The method of claim 128, wherein the additional therapy or therapeutic agent is selected from EGFR inhibitors, HER2 inhibitors, MEK inhibitors, RAF inhibitors, KRAS inhibitors, cytotoxic chemotherapeutics, angiogenesis inhibitors, and radiotherapy.

130. The method of any one of claims 106-129, wherein the cancer is colorectal cancer, lung cancer, thyroid cancer, breast cancer, ovarian cancer, bladder cancer, uterine cancer, prostate cancer, esophageal cancer, head and neck cancer, kidney cancer, liver cancer, pancreatic cancer, or stomach cancer.

131. The method of any one of claims 106-130, wherein the cancer is selected from the group consisting of breast cancer, ovarian cancer, bladder cancer, uterine cancer, prostate cancer, lung cancer, esophageal cancer, liver cancer, pancreatic cancer, and stomach cancer.

132. The method of any one of claims 106-131, wherein the cancer is selected from the group consisting of breast cancer, ovarian cancer, and colorectal cancer.

133. The method of any one of claims 106-132, wherein the cancer is selected from the group consisting of breast cancer and ovarian cancer.

134. The method of any one of claims 106-133, wherein the cancer is breast cancer.

135. The method of any one of claims 106-134, wherein the cancer is breast cancer selected from the group consisting of estrogen receptor (ER)-positive/hormone receptor (HR)-positive breast cancer, HER2-negative breast cancer; ER-positive/HR-positive breast cancer, HER2-positive breast 88 cancer; triple negative breast cancer (TNBC); and inflammatory breast cancer.

136. The method of any one of claims 106-134, wherein the cancer is breast cancer selected from the group consisting of endocrine resistant breast cancer, trastuzumab-resistant breast cancer, and breast cancer demonstrating primary or acquired resistance to CDK4/CDK6 inhibition.

137. The method of any one of claims 106-133, wherein the cancer is ovarian cancer.

138. The method of any one of claims 106-130, wherein the cancer is colorectal cancer.

139. A method for inhibiting mammalian cell proliferation, comprising contacting the mammalian cell with a compound of any one of claims 1-104, or a pharmaceutically acceptable salt thereof.

140. A method for inhibiting CDK2 activity in a mammalian cell, comprising contacting the mammalian cell with a compound of any one of claims 1-104, or a pharmaceutically acceptable salt thereof.

141. The method of claim 139 or 140, wherein the contacting occurs in vivo.

142. The method of claim 139 or 140, wherein the contacting occurs in vitro.

143. The method of any one of claims 139-142, wherein the mammalian cell is a mammalian cancer cell.

144. The method of any one of claims 139-143, wherein the mammalian cell has dysregulation of a CDK2 gene, a CDK2 protein, or expression or activity or level of any of the same.

145. The method of any one of claims 139-143, wherein the mammalian cell has dysregulation of a cyclin A2 gene, a cyclin A2 protein, or expression or activity or level of any of the same in a sample from the subject.

146. The method of any one of claims 139-145, wherein the mammalian cell has dysregulation of a cyclin E1 gene, a cyclin E1 protein, or expression or activity or level of any of the same.

147. The method of any one of claims 139-146, wherein the mammalian cell has dysregulation of a cyclin E2 gene, a cyclin E2 protein, or expression or activity or level of any of the same.
